(19) United States
(12) Patent Application Publication
(10) Pub. No.: US 2021/0114983 A1
(43) Pub. Date: Apr. 22, 2021

(54) Cathepsin C inhibitors

(71) Applicant: National Institute of Biological Sciences, Beijing, Beijing (CN)

(72) Inventors: Zhiyuan Zhang, Beijing (CN); Weijie Hou, Beijing (CN); Huan Sun, Beijing (CN)

(73) Assignee: National Institute of Biological Sciences, Beijing, Beijing (CN)

(21) Appl. No.: 17/137,458

(22) Filed: Dec. 30, 2020

Related U.S. Application Data
(63) Continuation of application No. PCT/CN2019/092488, filed on Jun. 24, 2019.

(30) Foreign Application Priority Data
Jun. 30, 2018 (CN) PCT/CN2018/093886

(51) Int. Cl. C07D 213/74; A61P 37/06; C07D 401/12; C07D 401/04; C07D 471/04; C07D 471/08; C07D 405/04; C07D 401/14; C07D 213/85

(52) U.S. Cl. CPC C07D 213/74; A61P 37/06; C07D 401/12; C07D 401/04; C07D 213/85; C07D 471/08; C07D 405/04; C07D 401/14; C07D 471/04

(57) ABSTRACT

Disclosed compounds, pharmaceutical compositions are used for inhibiting cathepsin C without inhibiting epidermal growth factor receptor (EGFR).

### PRIORITY

PCT/CN2018/093886, Filed: Jun. 30, 2018; Sub No.: 189308

### INTRODUCTION

Cathepsin C (CTSC) also known as dipeptidyl peptidase I (DPP-I) is a lysosomal exo-cysteine of the peptidase C1 family. Cathepsin C functions as a key enzyme in the activation of granule serine peptidases in inflammatory cells, such as elastase and cathepsin G in neutrophils cells and chymase and tryptase in mast cells. In many inflammatory diseases, such as rheumatoid arthritis, chronic obstructive pulmonary disease (COPD), inflammatory bowel disease, asthma, sepsis, and cystic fibrosis, a significant portion of the pathogenesis is caused by increased activity of some of these inflammatory proteases. Once activated by cathepsin C, the proteases are capable of degrading various extracellular matrix components, which can lead to tissue damage and chronic inflammation.

In drug screens we found that some irreversible kinase inhibitors also display inhibition activity against the peptidase cathepsin C. By computer modeling of cathepsin C crystal structure we determined structural features essential for anti-cathepsin C activity and anti-EGFR activity, respectively, and designed a novel class of specific cathepsin C inhibitors which do not target EGFR kinase activity. For example, we determined that a nitrogen atom of the pyrimidine ring of the EGFR inhibitor WZ4002 (WO2010129053) is not necessary for anti-cathepsin C activity, but crucial for maintaining the anti EGFR kinase activity. After developing initial leads we used SAR to develop a novel class of specific cathepsin C inhibitors disclosed here.

### SUMMARY OF THE INVENTION

The invention provides compounds, pharmaceutical compositions and related methods for inhibiting cathepsin C. In an aspect the invention provides a compound of formula:

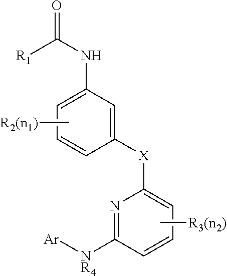

wherein:

X is O, S or NR5;

Ar is a substituted or unsubstituted 5-6 membered aryl or heteroaryl;

R1 is substituted or unsubstituted ethenyl or ethynyl;

R2 and R3 are independently optionally substituted, optionally hetero-, optionally cyclic C1-C18 hydrocarbyl, or optionally substituted heteroatom;

R4 and R5 are independently H, optionally substituted, optionally hetero-, optionally cyclic C1-C18 hydrocarbyl, or optionally substituted heteroatom; and

each of n1 and n2 is independently 0, 1, 2 or 3;

or a pharmaceutically acceptable salt, hydrate or stereoisomer the compound.

In embodiments:

X is O;

Ar is substituted or unsubstituted pyrrole, azole (e.g. pyrazole, imidazole, triazole, tetrazole, pentazole, oxazole, isoxazole, thiazole or isothiazole), furan, dioxole thiophene, dithiole or oxathiole, preferably 2-moieties, such as 2-azole, 2-pyrrole, 2-azole (e.g. 2-pyrazole, 2-imidazole, 2-oxazole, 2-isoxazole, 2-thiozole, or 2-isothiozole), 2-furan, 2-thiophene, 2-oxole, dioxole, or 2-thiole; or substituted or unsubstituted, phenyl, pyridine, pyran, diazine, oxazine, thiazine, dioxine dithiin or triazine;

R1 is ethenyl, ethynyl, propenyl, 2-methylpropenyl, propyne, each optionally fluorinated;

R2 and R3 are independently halogen (e.g. F, Cl, Br), CN, optionally substituted. C1-C4 alkyl, alkenyl, alkynyl or alkyloxy, each optionally fluorinated (e.g. Me, CF3, methoxy, ethynyl, dimethylamine, pyrrolidinyl, morpholinyl, piperidinyl, phenyl, 3-hydroxyl pyrrolidinyl, 3-hydroxyl piperidinyl, 4-methylpiperazinyl, phenyl); and/or

R4 and R5 are independently H, optionally substituted C1-C4 alkyl, alkenyl or alkynyl or CN, each optionally fluorinated (e.g. Me, CF3, benzyl).

In embodiments Ar is substituted with a substituent which forms a salt bridge with cathepsin C, particularly a cyclic or chain amine, wherein the nitrogen atom of the amine forms a salt bridge with cathepsin C, which helps maintain cathepsin C inhibitory activity, particularly wherein Ar comprises a structure:

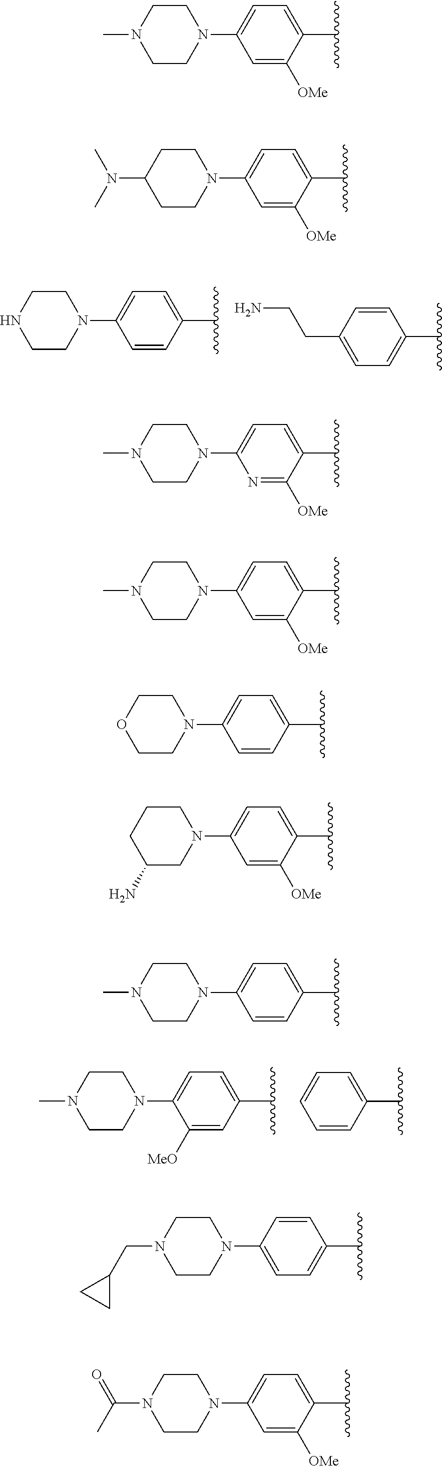

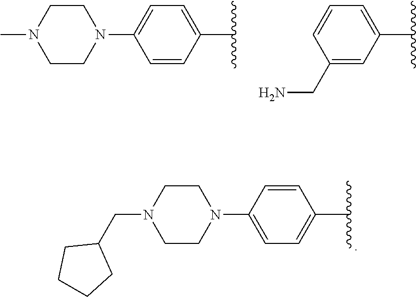 [SMILES: *c1ccc(N2CCN(C)CC2)cc1 ; *c1cccc(CN)c1 ; *c1ccc(N2CCN(CC3CCCC3)CC2)cc1]

In embodiments the substituted groups comprise 1-6, and preferably 1-3 substituents selected from: halogen, —R′, —OR′, ═O, ═NR′, ═N—OR′, —NR′R″, —SR′, —SiR′R″R″, —OC(O)R′, —C(O)R′, —CO2R′, —CONR′R″, —OC(O)NR′R″, —NR″C(O)R′, —NR′—C(O)NR″R′″, —NR′—SO2NR′″, —NR″CO2R′, —NH—C(NH2)=NH, —NR′C(NH2)=NH, —NH—C(NH2)=NR′, —S(O)R′, —SO2R′, —SO2NR′R″, —NR″SO2R, —CN and —NO2, —N3, —CH(Ph)2, perfluoro(C1-C4)alkoxy and perfluoro(C1-C4)alkyl, wherein R′, R″ and R′″ each independently refer to hydrogen, unsubstituted (C1-C8)alkyl and heteroalkyl, (C1-C8)alkyl and heteroalkyl substituted with one to three halogens, unsubstituted aryl, aryl substituted with one to three halogens, unsubstituted alkyl alkoxy or thioalkoxy groups, or aryl-(C1-C4)alkyl groups, wherein preferred substituents are selected from: halogen, —R′, —OR′, ═O, —NR′R″, —SR′, —SiR′R″R′″, —OC(O)R′, —C(O)R′, —CO2R′, —CONR′R″, —OC(O)NR′R″, —NR″C(O)R′, —NR″CO2R′, —NR′—SO2NR″R′″, —S(O)R′, —SO2R′, —SO2NR′R″, —NR″SO2R, —CN and —NO2, perfluoro(C1-C4)alkoxy and perfluoro(C1-C4)alkyl, where R′ and R″ are as defined above.

In embodiments the compound has a structure disclosed herein, e.g. in Table 1, 2 or 3.

In embodiments the compounds is a cathepsin C inhibitor (e.g. IC50 of less than 30, 10, 3 or 1 μM) and not an epidermal growth factor receptor (EGFR) inhibitor, wherein the EGFR IC50 is at least 3, 10, 100, or 1000 times higher than the cathepsin C IC50.

In another aspect the invention provides a pharmaceutical composition comprising a therapeutically effective amount of a subject compound in unit dosage form and one or more pharmaceutically acceptable excipients.

In another aspect the invention provides a method of using a subject compound or composition to inhibit cathepsin C comprising: administering to a cell or person in need thereof an effective amount of a subject compound or composition, or a prodrug thereof.

In another aspect the invention provides a method of using a subject compound or composition to treat inflammation comprising: administering to a cell or person in need thereof an effective amount of a subject compound or composition, or a prodrug thereof.

In embodiments the method further comprises the antecedent step of diagnosing the need thereof or the inflammation or the subsequent step of detecting a resultant cathepsin C inhibition or amelioration of the inflammation.

The invention encompasses all combination of the particular embodiments recited herein, as if each combination had been laboriously recited.

### BRIEF DESCRIPTION OF THE DRAWINGS

FIG. 1. Compound 28 inhibited the activation of CatC, NE and CatG, in a dose dependent manner in rat bone marrow.

FIG. 2. Compound 28 inhibited the activation of CatC, NE, Pr3 and CatG, in a dose dependent manner in mice bone marrow.

FIG. 3. Compound 28 inhibited the activation of CatC, NE, Pr3 and CatG, in a dose dependent manner in mice blood.

FIG. 4a. Compound 28 reduced the severity of acute pancreatitis: CatC activity in homogenates prepared from mice pancreas samples.

FIG. 4b. Compound 28 reduced the severity of acute pancreatitis: Pancreas histology representative photomicrographs.

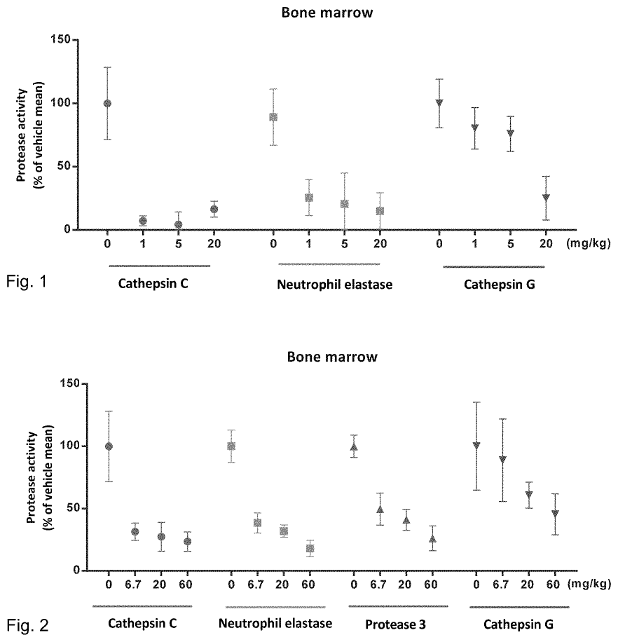

### DESCRIPTION OF PARTICULAR EMBODIMENTS OF THE INVENTION

The term “alkyl” refers to a hydrocarbon group selected from linear and branched saturated hydrocarbon groups of 1-18, or 1-12, or 1-6 carbon atoms. Examples of the alkyl group include methyl, ethyl, 1-propyl or n-propyl (“n-Pr”), 2-propyl or isopropyl (“i-Pr”), 1-butyl or n-butyl (“n-Ru”), 2-methyl-1-propyl or isobutyl (“i-Bu”), 1-methylpropyl or s-butyl (“s-Bu”), and 1,1-dimethylethyl or t-butyl (“t-Bu”). Other examples of the alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

Lower alkyl means 1-8, preferably 1-6, more preferably 1-4 carbon atoms; lower alkenyl or alkynyl means 2-8, 2-6 or 2-4 carbon atoms.

The term “alkenyl” refers to a hydrocarbon group selected from linear and branched hydrocarbon groups comprising at least one C═C double bond and of 2-18, or 2-12, or 2-6 carbon atoms. Examples of the alkenyl group may be selected from ethenyl or vinyl, prop-1-enyl, prop-2-enyl, 2-methylprop-1-enyl, but-1-enyl, but-2-enyl, but-3-enyl, butts-1,3-dienyl, 2-methylbuta-1,3-diene, hex-1-enyl, hex-2-enol, hex-3-enyl, hex-4-enyl, and hexa-1,3-dienyl groups.

The term “alkynyl” refers to a hydrocarbon group selected from linear and branched hydrocarbon group, comprising at least one C≡C triple bond and of 2-18, or 2-12, or 2-6 carbon atoms. Examples of the alkynyl group include ethynyl, 1-propynyl, 2-propynyl (propargyl), 1-butynyl, 2-butynyl, and 3-butynyl groups.

The term “cycloalkyl” refers to a hydrocarbon group selected from saturated and partially unsaturated cyclic hydrocarbon groups, comprising monocyclic and polycyclic (e.g., bicyclic and tricyclic) groups. For example, the cycloalkyl group may be of 3-12, or 3-8, or 3-6 carbon atoms. Even further for example, the cycloalkyl group may be a monocyclic group of 3-12, or 3-8, or 3-6 carbon atoms. Examples of the monocyclic cycloalkyl group include cyclopropyl, cyclobutyl, cyclopentyl, 1-cyclopent-1-enol, 1-cyclopent-2-enyl, 1-cyclopent-3-enyl, cyclohexyl, 1-cyclohex-1-enol, 1-cyclohex-2-enol, 1-cyclohex-3-enol, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e. partially unsaturated), but is not fully conjugated, and is not aromatic, as aromatic is defined herein.

The term “aryl” herein refers to a group selected from 5- and 6-membered carbocyclic aromatic rings, for example, phenyl; bicyclic ring systems such as 7-12 membered bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group is selected from 5- and 6-membered carbocyclic aromatic rings fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “halogen” or “halo” refers to F, Cl, Br or I.

The term “heteroalkyl” refers to alkyl comprising at least one heteroatom.

The term “heteroaryl” refers to a group selected from:

5- to 7-membered aromatic, monocyclic rings comprising 1, 2, 3 or 4 heteroatoms selected from N, O, and S, with the remaining ring atoms being carbon;

8- to 12-membered bicyclic rings comprising 1, 2, 3 or 4 heteroatoms, selected from N, O, and S, with the remaining ring atoms being carbon and wherein at least one ring is aromatic and at least one heteroatom is present in the aromatic ring; and

11- to 14-membered tricyclic rings comprising 1, 2, 3 or 4 heteroatoms, selected from N, O, and S, with the remaining ring atoms being carbon and wherein at least one ring is aromatic and at least one heteroatom is present in an aromatic ring.

For example, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-Chia-2,5-diazolyl, 1-Chia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “heterocyclic” or “heterocycle” or “heterocyclyl” refers to a ring selected from 4- to 12-membered monocyclic, bicyclic and tricyclic, saturated and partially unsaturated rings comprising at least one carbon atoms in addition to 1, 2, 3 or 4 heteroatoms, selected from oxygen, sulfur, and nitrogen. “Heterocycle” also refers to a 5- to 7-membered heterocyclic ring comprising at least one heteroatom selected from N, O, and S fused with 5-, 6-, and/or 7-membered cycloalkyl, carbocyclic aromatic or heteroaromatic ring, provided that the point of attachment is al the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e. partially unsaturated). The heterocycle may be substituted with oxo. The point of the attachment may be carbon or heteroatom in the heterocyclic ring. A heterocyle is not a heteroaryl as defined herein.

Examples of the heterocycle include, but not limited to, (as numbered from the linkage position assigned priority 1) 1-pyrrolidinyl, 2-pyrrolidinyl, 2,3-pyrazolidinyl, 1-piperidinyl, 2-piperidinyl 4-piperidinyl, 2,5-piperazinyl, pyranyl, 2-3-morpholinyl, oxirayl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 411-pyranyl, 1,4-dioxanyl, 1,3-dioxolanyl, pyrazolinyl, pyrazolidinyl, dithianyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, oxo-thiomorpholinyl and 1,1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicyclic aryl ring such as 7 to 12 membered bicyclic aryl ring systems as mentioned above, a fused tricyclic aryl ring such as 10 to 15 membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

In embodiments substituents are selected from optionally substituted heteroatom and optionally substituted, optionally hetero-, optionally cyclic C1-C18 hydrocarbyl, particularly wherein the optionally substituted, optionally hetero-, optionally cyclic C1-C18 hydrocarbyl is optionally-substituted, optionally hetero-, optionally cyclic alkyl, alkenyl or alkynyl, or optionally-substituted, optionally hetero-aryl; and/or the optionally substituted heteroatom is halogen, optionally substituted hydroxyl (such as alkoxy, aryloxy), optionally substituted acyl (such as formyl, alkanoyl, carbamoyl, carboxyl, amido), optionally substituted amino (such as amino, alkylamino, dialkylamino, amido, sulfamidyl), optionally substituted thiol (such as mercapto, alkylthiol, aryl thiol), optionally substituted sulfinyl or sulfonyl (such as alkylsulfinyl, arylsulfinyl, alkyl sulfonyl, arylsulfonyl), nitro, or cyano.

In embodiments, substituents are selected from: halogen, —R′, —OR′, ═O, ═NR′, ═N—OR′, —NR′R″, —SR′, —SiR′R″R′″, —OC(O)R′, —C(O)R′, —CO2R′, —CONR′R″, —OC(O)NR′R″, —NR″C(O)R′, —NR′—C(O)NR″R′″, —NR″—SO2NR′″, —NR″CO2R′, —NH—C(NH2)=NH, —NR′C(NH2)=NH, —NH—C(NH2)=NR′, —S(O)R′, —SO2R′, —SO2NR′R″, —NR″SO2R, —CN and —NO2, —N3, —CH(Ph)2, perfluoro(C1-C4)alkoxy and perfluoro(C1-C4)alkyl, in a number ranging from zero to three, with those groups having zero, one or two substituents being particularly preferred. R″, R″ and R′″ each independently refer to hydrogen, unsubstituted (C1-C8)alkyl and heteroalkyl, (C1-C8)alkyl and heteroalkyl substituted with one to three halogens, unsubstituted aryl, aryl substituted with one to three halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl, “alkyl” includes groups such as trihaloalkyl (e.g., —CF3 and —CH2CF3), and when the aryl group is 1,2,3,4-tetrahydronaphthalene, it may be substituted with a substituted or unsubstituted (C3-C7)spirocycloalkyl group. The (C3-C7)spirocycloalkyl group may be substituted in the same manner as defined herein for “cycloalkyl”.

Preferred substituents are selected from: halogen, —R′, —OR′, ═O, —NR′R″, —SiR′R″R′″, —OC(O)R′, —C(O)R′, —CO2R′, —CONR′R″, —OC(O)NR′R″, —NR″C(O)R′, —NR″CO2R′, —NR′—SO2NR″R′″, —S(O)R″, —SO2R″, —SO2NR′R″, —NR″SO2R, —CN and —NO2, perfluoro(C1-C4)alkoxy and perfluoro(C1-C4)alkyl, where R′ and R″ are as defined above.

Preferred substituents are disclosed herein and exemplified in the tables, structures, examples, and claims, and may be applied across different compounds of the invention, i.e. substituents of any given compound may be combinatorially used with other compounds.

In particular embodiments applicable substituents are independently substituted or unsubstituted heteroatom, substituted or unsubstituted, 0-3 heteroatom C1-C6 alkyl, substituted or unsubstituted, 0-3 heteroatom C2-C6 alkenyl, substituted or unsubstituted, 0-3 heteroatom C2-C6 alkenyl, or substituted or unsubstituted, 0-3 heteroatom C6-C14 aryl, wherein each heteroatom is independently oxygen, phosphorus, sulfur or nitrogen.

In more particular embodiments, applicable substituents are independently aldehyde, aldimine, alkanoyloxy, alkoxy, alkoxycarbonyl, alkyloxy, alkyl, amine, azo, halogens, carbamoyl, carbonyl, carboxamido, carboxyl, cyanyl, ester, halo, haloformyl, hydroperoxyl, hydroxyl, imine, isocyanide, iscyante, N-tert-butoxycarbonyl, nitrate, nitrile, nitrite, nitro, nitroso, phosphate, phosphono, sulfide, sulfonyl, sulfo, sulfhydryl, thiol, thiocyanyl, trifluoromethyl or trifluromethyl ether (OCF3).

The compounds may contain an asymmetric center and may thus exist as enantiomers. Where the compounds possess two or more asymmetric centers, they may additionally exist as diastereomers. Enantiomers and diastereomers fail within the broader class of stereoisomers. All such possible stereoisomers as substantially pure resolved enantiomers, racemic mixtures thereof, as well as mixtures of diastereomers are intended to be included. All stereoisomers of the compounds and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds contain olefin double bonds, unless specified otherwise, such double bonds are meant to include both E and Z geometric isomers.

Some of the compounds may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

It may be advantageous to separate reaction products from one another and/or from starting materials. The desired products of each step or series of steps is separated and; or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents. Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n-COOH, wherein n is selected from 0 to 4. Similarly, examples of pharmaceutically acceptable cations include, but are not limited to, sodium, potassium, calcium, aluminum, lithium, and ammonium.

In addition, if a compound is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

“Treating,” “treat,” or “treatment” refers to administering at least one compound and/or at least one stereoisomer thereof, and/or at least one pharmaceutically acceptable salt thereof to a subject in recognized need thereof.

An “effective amount” refers to an amount of at least one compound and/or at least one stereoisomer thereof, and/or at least one pharmaceutically acceptable salt thereof effective to “treat” a disease or disorder in a subject, and that will elicit, to some significant extent, the biological or medical response of a tissue, system, animal or human that is being sought, such as when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

The term “at least one substituent” includes, for example, from 1 to 4, such as from 1 to 3, further as 1 or 2, substituents. For example, “at least one substituent R¹⁶” herein includes from 1 to 4, such as from 1 to 3, further as 1 or 2, substituents selected from the list of R¹⁶ as described herein.

The subject compounds and stereoisomers thereof, and pharmaceutically acceptable salts thereof may be employed alone or in combination with at least one other therapeutic agent for treatment. In some embodiments, the compounds, stereoisomers thereof, and pharmaceutically acceptable salts thereof can be used in combination with at least one additional therapeutic agent. The compound and/or one pharmaceutically acceptable salt disclosed herein may be administered with the at least one other therapeutic agent in a single dosage form or as a separate dosage form. When administered as a separate dosage form, the at least one other therapeutic agent may be administered prior to, at the same time as, or following administration of the compound and/or one pharmaceutically acceptable salt disclosed herein.

Also provided is a composition comprising a subject compound and stereoisomers thereof, and pharmaceutically acceptable salts thereof, and at least one pharmaceutically acceptable carrier.

The composition comprising a subject compound and stereoisomers thereof, and pharmaceutically acceptable salts thereof can be administered in various known manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art.

The subject compounds and stereoisomers thereof, and pharmaceutically acceptable salts thereof can be administered orally in solid dosage forms, such as capsules, tablets, troches, dragées, granules and powders, or in liquid dosage forms, such as elixirs, syrups, emulsions, dispersions, and suspensions. The subject compounds and stereoisomers thereof, and pharmaceutically acceptable salts thereof disclosed herein can also be administered parenterally, in sterile liquid dosage forms, such as dispersions, suspensions or solutions. Other dosages forms that can also be used to administer the subject compounds and stereoisomers thereof, and pharmaceutically acceptable salts thereof disclosed herein as an ointment, cream, drops, transdermal patch or powder for topical administration, as an ophthalmic solution or suspension formation, i.e., eye drops, for ocular administration, as an aerosol spray or powder composition for inhalation or intranasal administration, or as a cream, ointment, spray or suppository tier rectal or vaginal administration.

Gelatin capsules containing the compound and/or the at least one pharmaceutically acceptable salt thereof disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage turns for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene gycols can be examples of suitable carriers for parenteral solutions. Solutions for parenteral administration may comprise a water soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol, a standard reference text in the art.

For administration by inhalation, the subject compounds and stereoisomers thereof, and pharmaceutically acceptable salts thereof may be conveniently delivered in the form of an aerosol spray presentation from pressurized packs or nebulisers. The subject compounds and stereoisomers thereof, and pharmaceutically acceptable salts thereof may also be delivered as powders, which may be formulated and the powder composition may be inhaled with the aid of an insufflation powder inhaler device. One exemplary delivery system for inhalation can be metered dose inhalation (MDI) aerosol, which may be formulated as a suspension or solution of a subject compound and stereoisomers thereof, and pharmaceutically acceptable salts thereof disclosed herein in at least one suitable propellant, selected, for example, from fluorocarbons and hydrocarbons.

For ocular administration, an ophthalmic preparation may be formulated with an appropriate weight percentage of a solution or suspension of the subject compound and stereoisomers thereof, and pharmaceutically acceptable salts thereof in an appropriate ophthalmic vehicle, such that the subject compound and stereoisomers thereof, and at least one pharmaceutically acceptable salts thereof is maintained in contact with the ocular surface for a sufficient time period to allow the compound to penetrate the corneal and internal regions of the eye.

Useful pharmaceutical dosage-forms for administration of the subject compounds and stereoisomers thereof, and pharmaceutically acceptable salts thereof disclosed herein include, but are not limited to, hard and soft gelatin capsules, tablets, parenteral injectables, and oral suspensions.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, a large number of unit capsules can be prepared by filling standard two-piece hard gelatin capsules each with, for example, 100 milligrams of the subject compound and stereoisomers thereof, and pharmaceutically acceptable salt thereof disclosed herein in powder, 150 milligrams of lactose, 50 milligrams of cellulose, and 6 milligrams magnesium stearate.

In some embodiments, a mixture of the compound, stereoisomers thereof, and pharmaceutically acceptable salts thereof a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, a large number of tablets can be prepared by conventional procedures so that the dosage unit comprises, for example, 100 milligrams of the compound, stereoisomers thereof, and pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, and pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, stereoisomers thereof, and pharmaceutically acceptable salts thereof are administered stepwise or in conjunction with at least one other therapeutic agent. When drugs are administered in physical combination, the dosage form and administration route should be selected depending on the compatibility of the combined drugs. Thus the term coadministration is understood to include the administration of at least two agents concomitantly or sequentially, or alternatively as a fixed dose combination of the at least two active components.

The compounds, stereoisomers thereof, and pharmaceutically acceptable salt thereof disclosed herein can be administered as the sole active ingredient or in combination with at least one second active ingredient.

The subject compounds are incorporated into pharmaceutical compositions or formulations. The compositions will contain pharmaceutically acceptable diluents and/or carriers, i.e. diluents or carriers that are physiologically compatible and substantially free from pathogenic impurities. Suitable excipients or carriers and methods for preparing administrable compositions are known or apparent to those skilled in the art and are described in more detail in such publications as Remington's Pharmaceutical Science, Mack Publishing Co, NJ (1991). The compositions may also be in the form of controlled release or sustained release compositions as known in the art. For many applications the subject compounds are administered for morning/daytime dosing, with off period at night.

The subject compounds may be used per se, or in the form of their pharmaceutically acceptable salts, such as hydrochlorides, hydrobromides, acetates, sulfates, citrates, carbonates, trifluoroacetates and the like. When compounds contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When compounds contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as alginate and the like, and salts of organic acids like glucuronic or galacturonic acids and the like (see, for example, Berge et al, “Pharmaceutical Salts”, Journal of Pharmaceutical Science, 1977, 66, 1-19).

The neutral forms of the compounds may be regenerated by contacting the salt with a base or acid, and isolating the parent compound in the conventional manner. The parent form of the compound differs from the various salt forms in certain physical properties, such as solubility in polar solvents, but otherwise the salts are equivalent to the parent form of the compound for the purposes of this invention.

In addition to salt forms, this invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity.

Certain compounds of the invention can exist in unsolvated forms as well as solvated forms, including hydrated forms. In general, the solvated forms are equivalent to unsolvated forms and are intended to be encompassed within the scope of the present invention. Certain compounds of the invention may exist in multiple crystalline or amorphous forms. In general, all physical forms are equivalent for the uses contemplated by the present invention and are intended to be within the scope of the invention.

Some of the subject compounds possess asymmetric carbon atoms (optical centers) or double bonds; the racemates, diastereomers, geometric isomers and individual isomers are all intended to be encompassed within the scope of the present invention.

The compounds of the invention may also contain unnatural proportions of atomic isotopes at one or more of the atoms that constitute such compounds, such as deuterium, e.g. CD₃, CD₂H or CDH₂ in place of methyl. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

The compounds are generally administered in a “therapeutically effective amount”, i.e. the amount of the subject compound that will elicit the biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

The contacting is generally effected by administering to the subject an effective amount of one or more compounds having the general formula 1 (supra), including the various embodiments described above. Generally administration is adjusted to achieve a therapeutic dosage of about 0.1 to 50, preferably 0.5 to 10, more preferably 1 to 10 mg/kg, though optimal dosages are compound specific, and generally empirically determined for each compound.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the mimetic is usually a minor component (from about 0.1 to about 50% by weight or preferably from about 1 to about 40% by weight) with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

Synthesis:

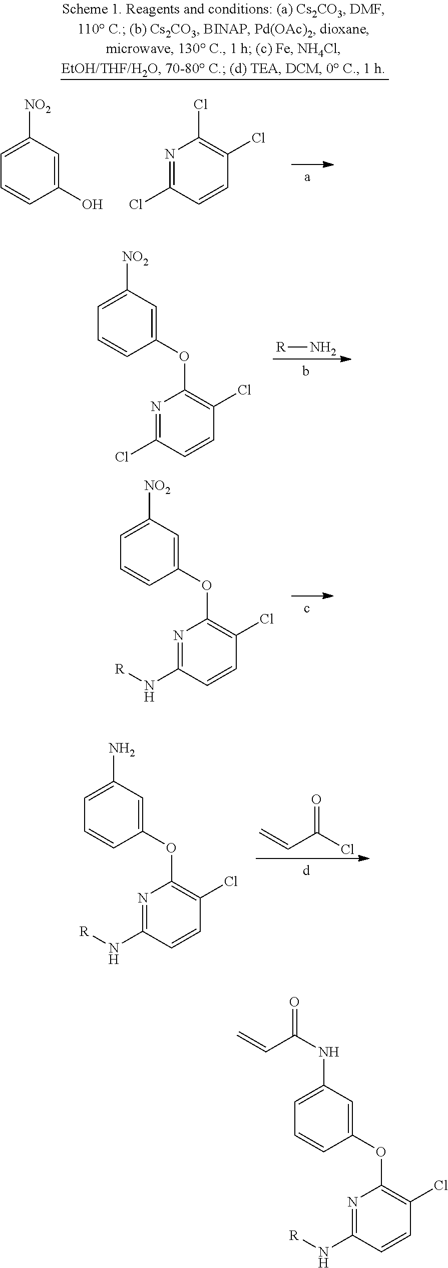

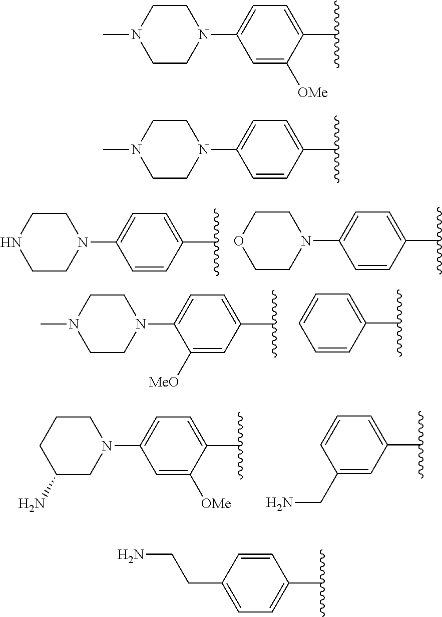

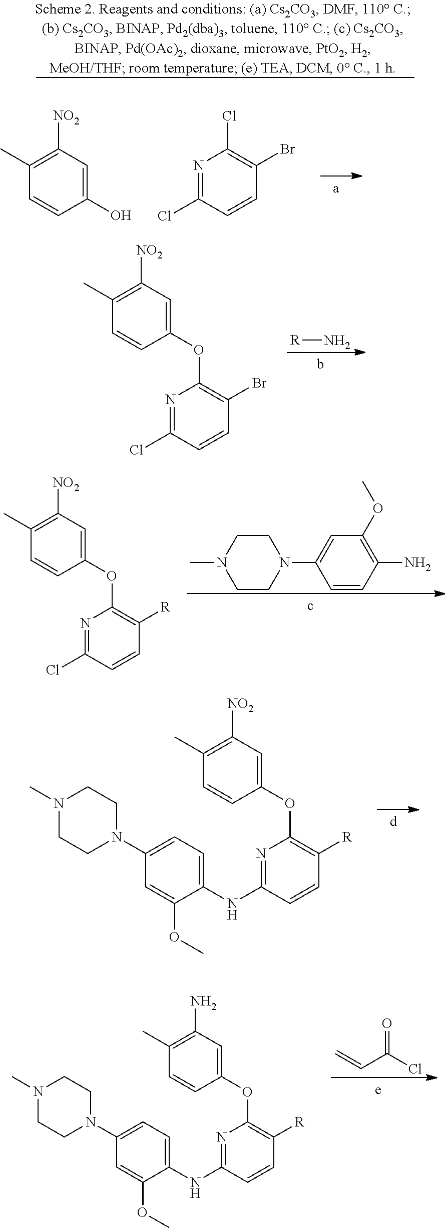

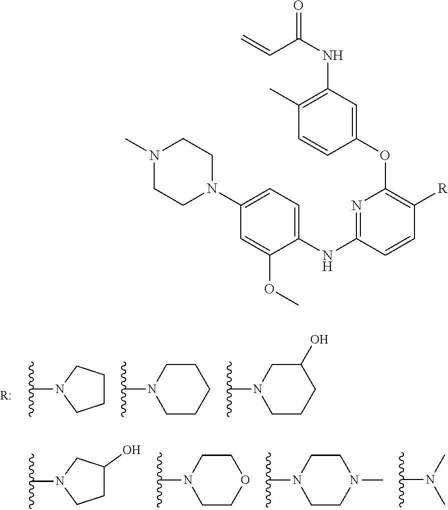

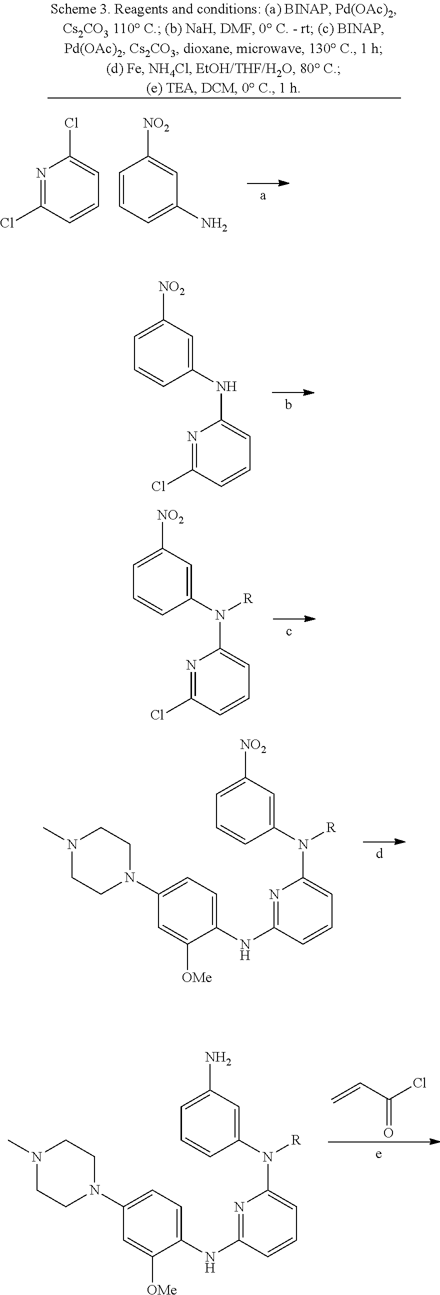

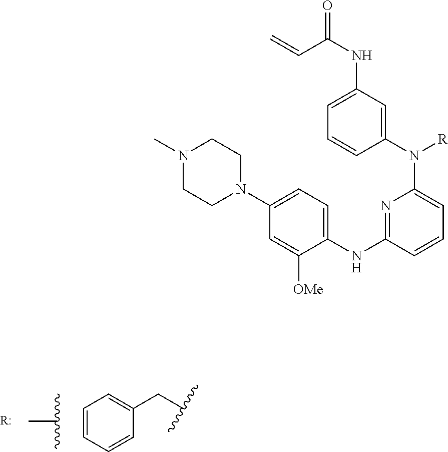

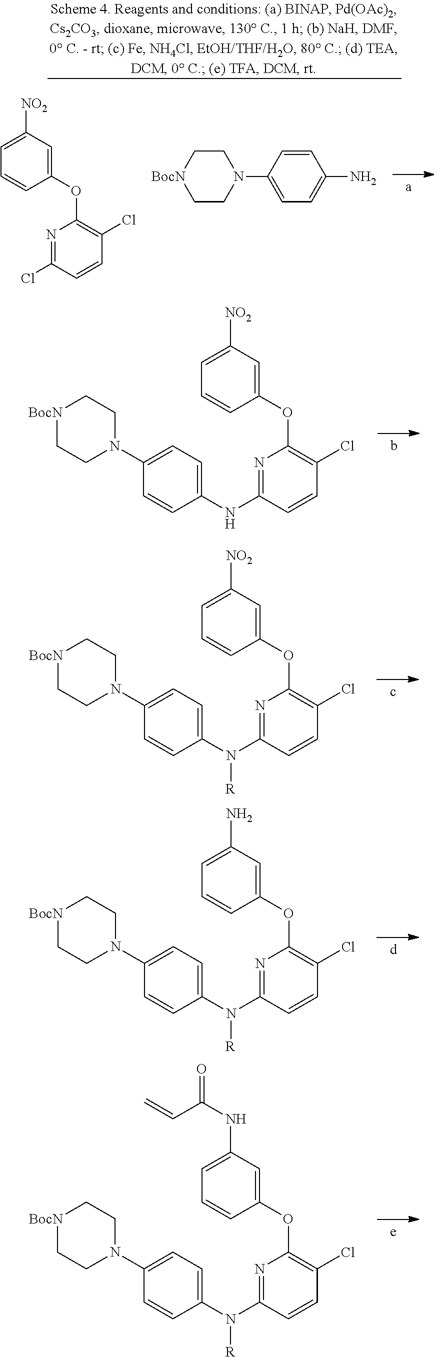

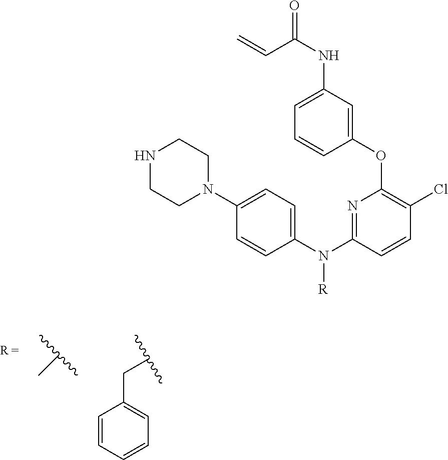

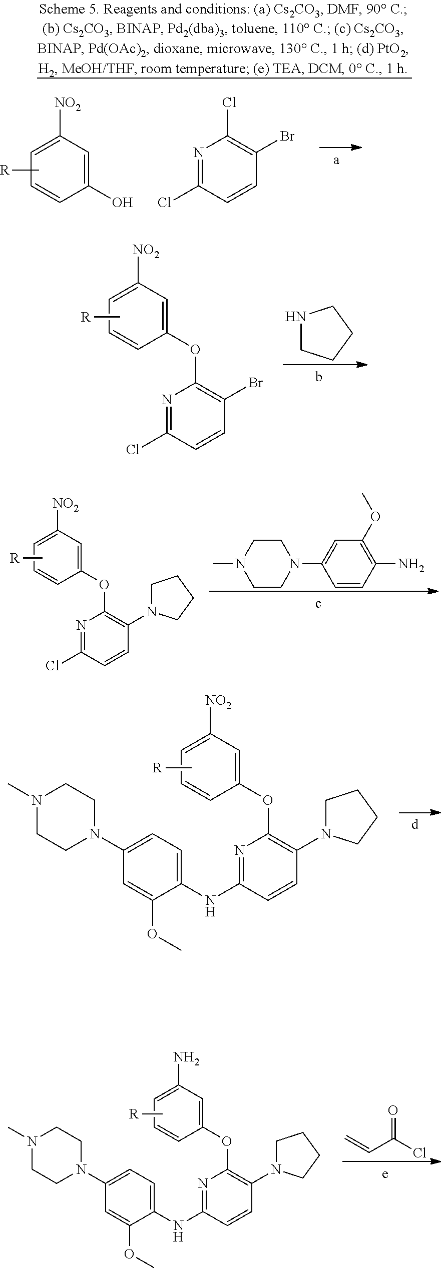

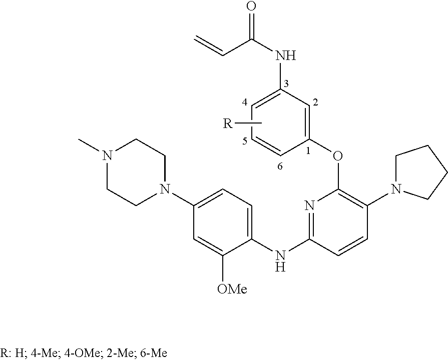

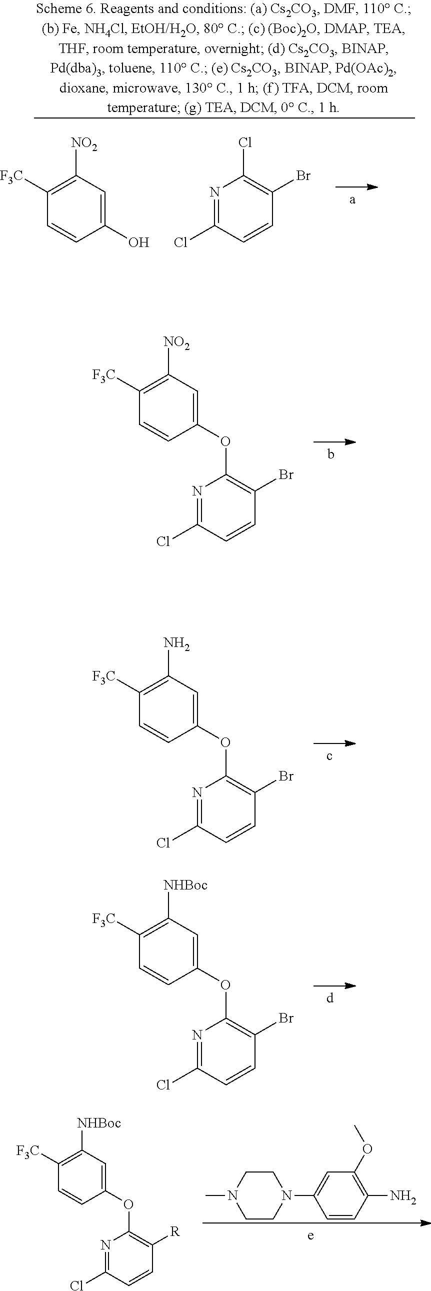

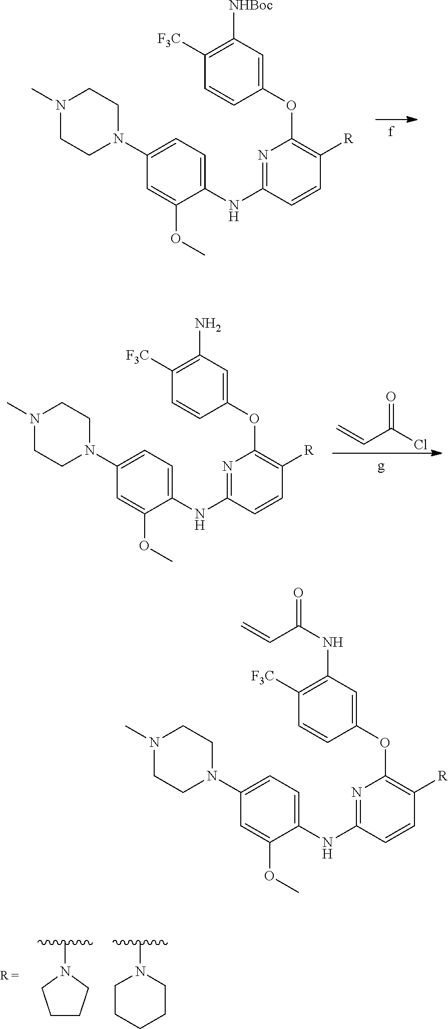

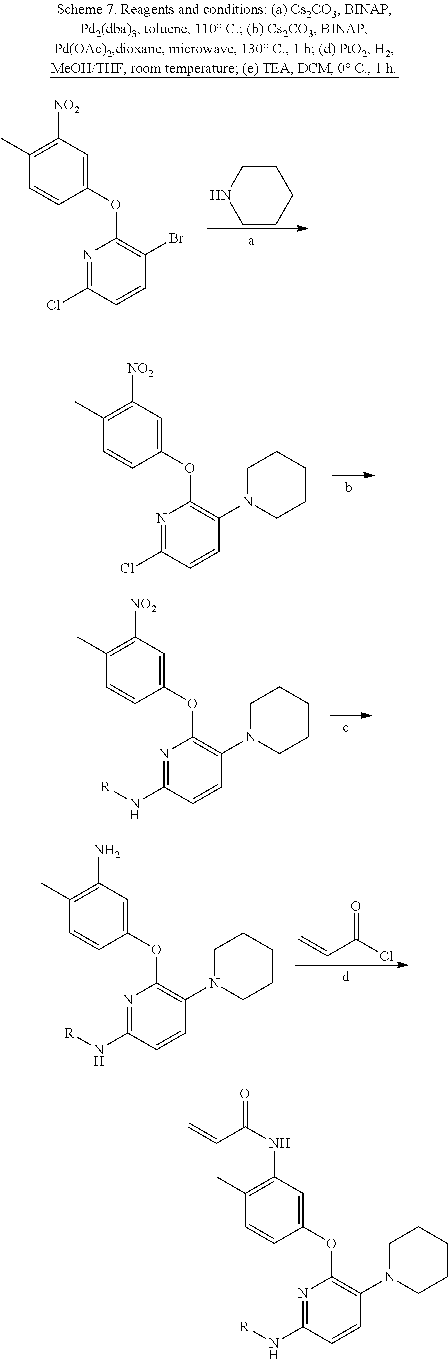

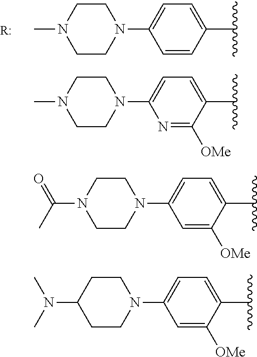

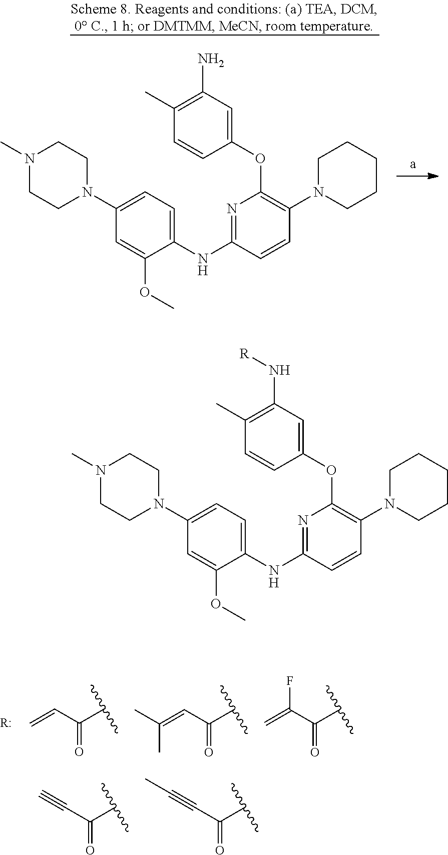

### Example 1: Synthesis of N-(5-((3-chloro-6-((2-methoxy-4-(4-methylpiperazin-1-yl)phenyl)amino)pyridin-2-yl)oxy)-2-methylphenyl)acrylamide

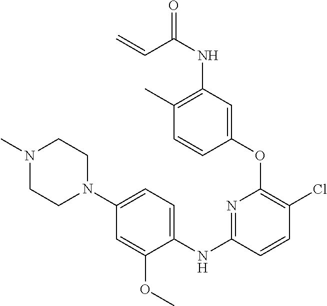 [SMILES: C=CC(=O)Nc1cc(Oc2nc(Nc3ccc(N4CCN(C)CC4)cc3OC)ccc2Cl)ccc1C]

### Step a: Synthesis of 3,6-dichloro-2-(3-nitrophenoxy)pyridine (Intermediate 1a)

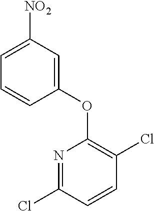 [SMILES: O=[N+]([O-])c1cccc(Oc2nc(Cl)ccc2Cl)c1]

Potassium carbonate (1.52 g, 10.96 mmol) and 2,3,6-trichloropyridine (1.0 g, 5.48 mmol) were added to the solution of 3-nitrophenol (763 mg, 5.48 mmol) in DMF (20 mL). The reaction mixture was heated to 100° C. fir 4 h. The reaction mixture was filtered, the filtrate was dilute with ethyl acetate and washed with brine three times. The organic layer wad dried over anhydrous sodium sulfate and concentrated, the residue product was purified by silica gel chromatograph to afford 1.02 g of white solid (yield: 65%). ¹H NMR 400 MHz (CDCl₃) δ 8.14-8.11 (m, 1H), 8.06 (t, 2.4 Hz, 1H), 7.75 (d, J=8.0 Hz, 1H), 7.60 (t, =8.0 Hz, 1H), 7.55-7.52 (m, 1H), 7.08 (d, J=8.0 Hz, 1H), LC-MS m/z: 285.02 (M+1)

### Step b: Synthesis of 5-chloro-N-(2-methoxy-4-(4-methylpiperazin-1-yl)phenyl)-6-(3-nitrophenoxy)pyridin-2-amine (Intermediate 1b)

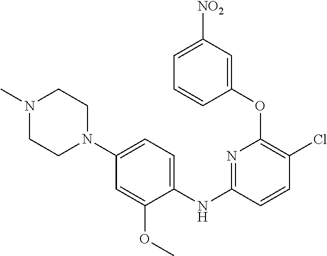 [SMILES: COc1cc(N2CCN(C)CC2)ccc1Nc1ccc(Cl)c(Oc2cccc([N+](=O)[O-])c2)n1]

A dioxane solution of 3,6-dichloro-2-(3-nitrophenoxy)pyridine (300 mg, 1.05 mmol), Pd(OAc)₂ (24 mg, 0.11 mmol), BINAP (98 mg, 0.16 mmol), cesium carbonate (515 mg, 1.58 mmol) and 2-methoxy-4-(4-methylpiperazin-1-yl)aniline (256 mg, 1.16 mmol) was added into a 20 mL microwave reaction vessel and capped. The reaction mixture was stirred for 1 hour at 130° C. using a biotage optimizer reactor. Water and ethyl acetate were added to the reaction solution, separated and then the aqueous phase was extracted with ethyl acetate. The combined organic phase was washed with saturated brine, then dried over anhydrous sodium sulfate. The organic phase was filtered off and then concentrated under reduced pressure. The obtained residue was purified by silica gel chromatograph to afford 309 mg of brown solid (yield: 62.6%). ¹H NMR 400 MHz (DMSO-d₆) δ 8.23 (s, 1H), 8.15-8.12 (m, 1H), 8.00 (t, =2.4 Hz, 1H), 7.73 (t, J=8.4 Hz, 1H), 7.68-7.65 (m, 1H), 7.65 (d, J=8.8 Hz, 1H), 7.07 (d, J=8.8 Hz, 1H), 6.59 (d, J=8.4 Hz, 1H), 6.50 (d, J=2.4 Hz, 1H), 5.97 (dd, J=2.4, 8.8 Hz, 1H), 3.74 (s, 3H), 3.00 (t, J=4.8 Hz, 4H), 2.43 (t, =4.8 Hz, 4H), 2.21 (s, 3H). LC-MS m/z: 470.36 (M+1).

### Step c: Synthesis of 6-(3-aminophenoxy)-5-chloro-N-(2-methoxy-4-(4-methylpiperazin-1-yl)phenyl)pyridin-2-amine (Intermediate 1c)

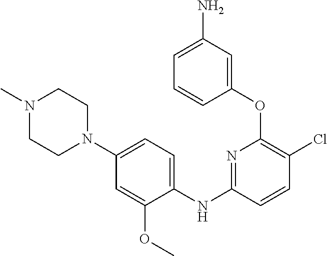 [SMILES: COc1cc(N2CCN(C)CC2)ccc1Nc1ccc(Cl)c(Oc2cccc(N)c2)n1]

5-chloro-N-(2-methoxy-4-(4-methylpiperazin-1-yl)phenyl)-6-(3-nitrophenoxy)pyridin-2-amine (305 mg, 0.65 mmol) was dissolved in ethanol (2 ml), tetrahydrofuran (2 mL) and water (2 mL) was added. Iron powder (363 mg, 6.49 mmol) and ammonium chloride (344 mg, 6.49 mmol) were then added, and the resulting mixture was heated to 75° C. for 4 hours. The reaction mixture was cooled to room temperature and filtered through celite. The solvent was removed in vacuo, and the resulting residue was basified with saturated sodium bicarbonate and extracted with ethyl acetate three times. The combined organic phase was dried with anhydrous sodium sulfate, concentrated, and purified by silica gel chromatograph with dichloromethane-methanol to afford 240 mg of brown solid (yield: 83.9%). ¹H NMR 400 MHz (DMSO-d₆) δ 8.07 (s, 1H), 7.57 (d, J=8.4 Hz, 1H), 7.41 (d, J=8.8 Hz, 1H), 7.03 (t, J=8.0 Hz, 1H), 6.54 (d, J=2.4 Hz, 1H), 6.52 (d, J=8.4 Hz, 1H), 6.42 (dd, =2.4, 8.0 Hz, 1H), 6.27 (t, =2.4 Hz, 1H), 6.23-6.17 (m, 1H), 5.21 (s, 3.77 (s, 3.04 (t, =4.8 Hz, 4H), 2.44 (t, J=4.8 Hz, 4H), 2.22 (s, 3H). LC-MS m/z: 440.33 (M+1).

### Step d: Synthesis of N-(3-((3-chloro-6-((2-methoxy-4-(4-methylpiperazin-1-yl)phenyl)amino)pyridin-2-yl)oxy)phenyl)acrylamide

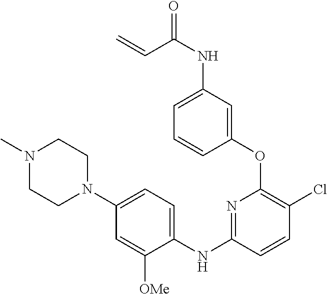 [SMILES: C=CC(=O)Nc1cccc(Oc2nc(Nc3ccc(N4CCN(C)CC4)cc3OC)ccc2Cl)c1]

### N-(3-((3-chloro-6-((2-methoxy-4-(4-methylpiperazin-1-yl)phenyl)amino)pyridin-2-yl)oxy)phenyl)acrylamide

Acryloyl chloride (14 mg, 0.16 mmol) was added dropwise to a solution of 6-(3-aminophenoxy)-5-chloro-N-(2-methoxy-4-(4-methylpiperazin-1-yl)phenyl)pyridin-2-amine (63 mg, 0.14 mmol) and triethylamine (30 μl, 0.21 mmol) in dichloromethane (2 mL) at 0° C. The reaction mixture was stirred at 0° C. for 1 h. Water and dichloromethane were added to the reaction solution, separated and then the aqueous phase was extracted with dichloromethane twice. The combined organic phase was washed with brine, then dried over anhydrous sodium sulfate. The organic phase was filtered off and then concentrated under reduced pressure. The obtained residue was dissolved in dichloromethane (2 ml) and TFA (0.25 ml) to afford the TFA salt of title compound. Dichloromethane and excess TFA was removed in vacuo and the residue dissolved in methanol and purified by reverse-phase HPLC eluting with acetonitrile containing 0.15‰ TFA. Fractions containing product were concentrated in vacuo to remove the solvent to give title compound (20 mg) as a gray solid. ¹H NMR (400 MHz, DMSO-d₆) δ 10.30 (s, 1H), 9.71 (bs, 1H), 8.23 (s, 1H), 7.64 (d, J=8.8 Hz, 1H), 7.61 (d, J=8.0 Hz, 1H), 7.44 (d, J=2.4 Hz, 1H), 7.40-7.35 (m, 2H), 6.86 (dd, J=8.0, 2.4 Hz, 1H), 6.64 (d, J=8.4 Hz, 1H), 6.69 (d, J=2.4 Hz, 1H), 6.42 (dd, J=16.8, 10.0 Hz, 1H), 6.26 (dd, J=16.8, 2.0 Hz, 1H), 6.07 (dd, J=8.8, 2.8 Hz, 1H), 5.77 (dd, J=10.0, 2.0 Hz, 1H), 3.78 (s, 3H), 3.66 (bs, 2H), 3.48 (bs, 2H), 3.13 (bs, 2H), 2.83 (bs, 2H), 2.86 (s, 3H). HRMS C₂₆H₂₈ClN₅O₃ (M+H)⁺ calculated mass, 494.1959; found, 494.1972.

### Example 2: Synthesis of N-(5-((6-((2-methoxy-4-(4-methylpiperazin-1-yl)phenyl)amino)-3-(piperidin-1-yl)pyridin-2-yl)oxy)-2-methylphenyl)acrylamide

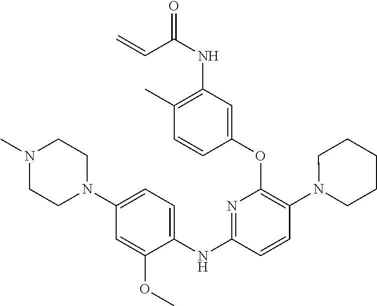 [SMILES: C=CC(=O)Nc1cc(Oc2nc(Nc3ccc(N4CCN(C)CC4)cc3OC)ccc2N2CCCCC2)ccc1C]

### Step a: Synthesis of 3-bromo-6-chloro-2-(1-methyl-3-nitrophenoxy)pyridine (Intermediate 2a)

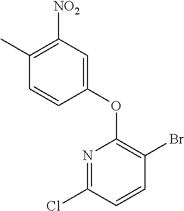 [SMILES: Cc1ccc(Oc2nc(Cl)ccc2Br)cc1[N+](=O)[O-]]

Caesium carbonate (14.4 g, 44.07 mmol) and 3-bromo-2,6-dichloropyridine (5.0 g, 22.04 mmol) were added to the solution of 4-methyl-3-nitrophenol (3.4 g, 22.04 mmol) in DMF (50 mL). The reaction mixture was heated to 90° C. for 8 h. The reaction mixture was filtered, the filtrate was dilute with ethyl acetate and washed with brine three times. The organic layer wad dried over anhydrous sodium sulfate and concentrated, the crude product was purified by silica gel chromatograph to afford 5.31 g of a white solid (yield: 70%). ¹H NMR 400 MHz (DMSO-d₆) δ 8.26 (d, J=8.0 Hz, 1H), 7.91 (d, J=2.4 Hz, 1H), 7.60-7.53 (m, 2H), 7.27 (d, J=8.0 Hz, 1H), 2.53 (s, 3H); LC-MS m/z: 343.03 (M+1).

### Step b: Synthesis of 6-chloro-2-(4-methyl-3-nitrophenoxy)-3-(piperidin-1-yl)pyridine (Intermediate 2b)

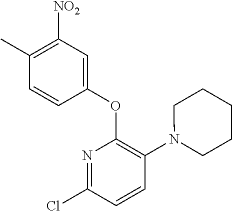 [SMILES: Cc1ccc(Oc2nc(Cl)ccc2N2CCCCC2)cc1[N+](=O)[O-]]

Piperidine (94 mg, 1.10 mmol) in anhydrous toluene (5 mL) was treated with 3-bromo-6-chloro-2-(4-methyl-3-nitrophenoxy)pyridine (345 mg, 1.00 mmol), Pd₂(dba)₃ (95 mg, 0.10 mmol), BINAP (65 mg, 0.10 mmol) and caesium carbonate (491 mg, 1.51 mmol). The reaction mixture was heated to 100° C. overnight. The mixture was allowed to cool to ambient temperature, diluted with ethyl acetate, washed with brine, dried over anhydrous sodium sulfate, and concentrated under reduced pressure. The residue was purified by silica gel chromatograph to provide the title compound (110 mg, 31%) as a brown oil. ¹H NMR 400 MHz (CDCl₃) δ 7.79 (d, J=2.0 Hz, 1H), 7.34-7.33 (m, 2H), 7.24-7.22 (m, 1H), 7.02 (d, J=8.0 Hz, 1H), 3.05 (t, J=4.2 Hz, 4H), 2.61 (s, 3H), 1.72-1.69 (m, 4H), 1.61-1.56 (m, 2H). LC-MS m/z: 348.28 (M+1).

### Step c: Synthesis of N-(2-methoxy-4-(4-methylpiperazin-1-yl)phenyl)-6-(4-methyl-3-nitrophenoxy)-5-(piperidin-1-yl)pyridin-2-amine (Intermediate 2c)

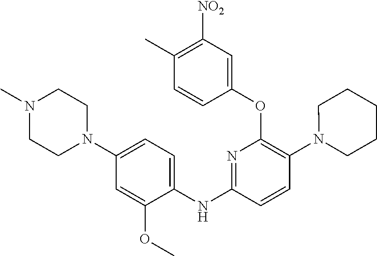 [SMILES: COc1cc(N2CCN(C)CC2)ccc1Nc1ccc(N2CCCCC2)c(Oc2ccc(C)c([N+](=O)[O-])c2)n1]

A dioxane solution of 6-chloro-2-(4-methyl-3-nitrophenoxy)-3-(piperidin-1-yl)pyridine (100 mg, 0.29 mmol), Pd(OAc)₂ (6.5 mg, 0.029 mmol), BINAP (27 mg, 0.043 mmol), cesium carbonate (141 mg, 0.43 mmol) and 2-methoxy-4-(4-methylpiperazin-1-yl)aniline (70 mg, 0.32 mmol) was added into a 5 mL microwave reaction vessel and capped. The reaction mixture was reacted at 130° C. for 1 hour using a biotage optimizer reactor. Water and ethyl acetate were added to the reaction solution, separated and then the aqueous phase was extracted with ethyl acetate. The combined organic phase was washed with saturated brine, then dried over anhydrous sodium sulfate. The organic phase was filtered off and then concentrated under reduced pressure. The obtained residue was purified by silica gel chromatography to afford the brown solid (69 mg, 45%), ¹H NMR 400 MHz (DMSO-d₆) δ 7.73 (d, J=2.4 Hz, 1H), 7.61 (s, 1H), 7.54 (d, J=8.8 Hz, 1H), 7.40 (dd, J=8.4, 4.2 Hz, 1H), 7.31 (d, J=8.4 Hz, 1H), 7.18 (d, J=8.8 Hz, 1H), 6.56 (d, J=8.4 Hz, 1H), 6.51 (d, J=2.8 Hz, 1H), 5.98 (dd, J=8.8, 2.4 Hz, 1H), 3.75 (s, 3H), 3.01 (t, J=5.2 Hz, 4H), 2.90 (t, J=5.2 Hz, 4H), 2.55 (s, 3H), 2.44 (t, J=4.8 Hz, 4H), 2.22 (s, 3H), 1.62-1.56 (m, 4H), 1.50-1.45 (m, 2H). LC-MS m/z: 533.42 (M+1).

### Step d: Synthesis of 6-(3-amino-4-methylphenoxy)-N-(2-methoxy-4-(4-methylpiperazin-1-yl)phenyl)-5-(piperidin-1-yl)pyridin-2-amine (Intermediate 2d)

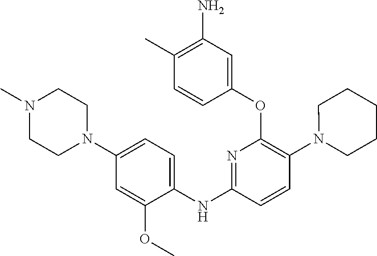 [SMILES: COc1cc(N2CCN(C)CC2)ccc1Nc1ccc(N2CCCCC2)c(Oc2ccc(C)c(N)c2)n1]

Platinum(IV) oxide (6 mg) was added to the solution of N-(2-methoxy-4-(4-methylpiperazin-1-yl)phenyl)-6-(4-methyl-3-nitrophenoxy)-5-(piperidin-1-yl)pyridin-2-amine (60 mg, 0.11 mmol) in methanol/tetrahydrofuran (2/2 ml). The reaction mixture was stirred under hydrogen overnight. The reaction mixture was filtrated through celite and concentrated to afford the title product, which was used without further purification. ¹H NMR (400 MHz, DMSO-d₆) δ 7.43-7.41 (m, 2H), 7.21 (d, J=8.4 Hz, 1H), 6.91 (d, J=8.8 Hz, 1H), 6.51 (d, J=2.8 Hz, 1H), 6.46 (d, J=8.4 Hz, 1H), 6.33 (d, J=2.4 Hz, 1H), 6.17 (dd, J=2.4, 8.0 Hz, 1H), 6.04 (dd, J=2.4, 8.8 Hz, 1H), 4.92 (s, 2H), 3.76 (s, 3H), 3.02 (t, J=4.8 Hz, 4H), 2.90 (t, J=4.8 Hz, 4H), 2.26 (s, 3H), 2.08 (s, 3H), 1.64-1.59 (m, 1.51-1.46 (m, 2H). LC-MS m/z: 503.32 (M+1).

### Step e: Synthesis of N-(5-((6-((2-methoxy-4-(4-methylpiperazin-1-yl)phenyl)amino)-3-(piperidin-1-yl)pyridin-2-yl)oxy)-2-methylphenyl)acrylamide

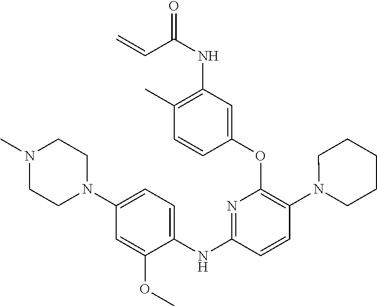 [SMILES: C=CC(=O)Nc1cc(Oc2nc(Nc3ccc(N4CCN(C)CC4)cc3OC)ccc2N2CCCCC2)ccc1C]

Acryloyl chloride (8 mg, 0,088 mmol) was added dropwise to a solution of 6-(3-amino-4-methylphenoxy)-N-(2-methoxy-4-(4-methylpiperazin-1-yl)phenyl)-5(piperidin-1-yl)pyridin-2-amine (40 mg, 0.08 mmol) and triethylamine (12 mg, 0.12 mmol) in anhydrous dichloromethane (2 mL) at 0° C. The reaction mixture was stirred at 0° C. for 1 hour. Water and dichloromethane were added to the reaction solution, separated and the aqueous phase was extracted with dichloromethane twice. The combined organic phase was washed with saturated brine, then dried over anhydrous sodium sulfate. The organic phase was filtered off and then concentrated under reduced pressure. The obtained residue was dissolved in dichloromethane (2 ml) and TFA (0.25 ml) to afford the TFA salt of title compound. Dichloromethane and excess TFA was removed in vacuo and the residue dissolved in methanol and purified by reverse-phase HPLC eluting with acetonitrile containing 0.15‰ TFA. Fractions containing product were concentrated in vacuo to remove the solvent to give title compound (15 mg) as a gray solid.

Structure of Compounds and HNMR/Mass Spectrum Data

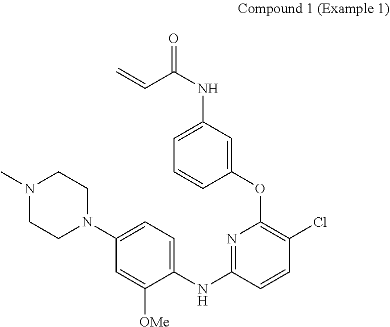 [SMILES: C=CC(=O)Nc1cccc(Oc2nc(Nc3ccc(N4CCN(C)CC4)cc3OC)ccc2Cl)c1]

### N-(3-((3-chloro-6-((2-methoxy-4-(4-methylpiperazin-1-yl)phenyl)amino)pyridin-2-yl)oxy)phenyl)acrylamide

¹H NMR (400 MHz, DMSO-d₆) δ 10.30 (s, 1H), 9.71 (bs, 1H), 8.23 (s, 1H), 7.64 (d, J=8.8 Hz, 1H), 7.61 (d, J=8.0 Hz, 1H), 7.44 (t, J=2.4 Hz, 1H), 7.40-7.35 (m, 2H), 6.86 (dd, J=8.0, 2.4 Hz, 1H), 6.64 (d, J=8.4 Hz, 1H), 6.69 (d, J=2.4 Hz, 1H), 6.42 (dd, J=16.8, 10.0 Hz, 1H), 6.26 (dd, J=16.8, 2.0 Hz, 1H), 6.07 (dd, J=8.8, 2.8 Hz, 1H), 5.77 (dd, J=10.0, 2.0 Hz, 1H), 3.78 (s, 3H), 3.66 (bs, 2H), 3.48 (bs, 2H), 3.13 (bs, 2H), 2.83 (bs, 2H), 2.86 (s, 3H). HRMS C₂₆H₂₈ClN₅O₃ (M+H)⁺ calculated mass, 494.1959; found, 494.1972.

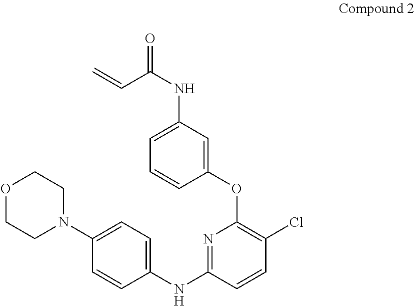 [SMILES: C=CC(=O)Nc1cccc(Oc2nc(Nc3ccc(N4CCOCC4)cc3)ccc2Cl)c1]

### N-(3-((3-chloro-6-((4-morpholinophenyl)amino)pyridin-2-yl)oxy)phenyl)acrylamide

Prepared according to example 1 (scheme 1) using 4-morpholinoaniline in step b to afford the title compound (20 mg) as a gray solid. ¹H NMR (400 MHz, DMSO-d₆) δ 10.31 (s, 1H), 9.09 (s, 1H), 7.67-7.61 (m, 2H), 7.54 (t, J=2.4 Hz, 1H), 7.42 (t, J=8.0 Hz, 1H), 7.13 (bs, 2H), 6.89 (dd, J=8.0, 2.4 Hz, 1H), 6.67 (bs, 2H), 6.47 (s, 1H), 6.42 (dd, J=16.8, 10.0 Hz, 1H), 6.25 (dd, J=16.8, 2.0 Hz, 1H), 5.77 (dd, J=10.0, 2.0 Hz, 1H), 3.73 (bs, 4H), 2.98 (bs, 4H). HRMS C₂₄H₂₄ClN4O₃ (M+H)⁺ calculated mass, 451.1537; found, 451.1521.

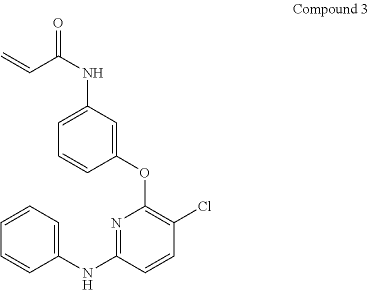 [SMILES: C=CC(=O)Nc1cccc(Oc2nc(Nc3ccccc3)ccc2Cl)c1]

### N-(3-((3-chloro-6-(phenylamino)pyridin-2-yl)oxy)phenyl)acrylamide

Prepared according to example 1 (scheme 1) using aniline in step b. In step d, the crude was purified by silica gel chromatography to afford the title compound (22 mg) as a gray solid. ¹H NMR (400 MHz, CDCl₃) δ 7.52 (dd, J=8.4, 0.8 Hz, 1H), 7.54-7.52 (m, 1H), 7.46 (s, 1H), 7.38 (t, J=8.4 Hz, 1H), 7.31 (s, 1H), 7.26 (s, 1H), 7.19-7.13 (m, 4H), 6.98-6.95 (m, 2H), 6.46-6.40 (m, 2H), 6.22 (dd, J=16.8, 10.0 Hz, 1H), 5.77 (d, J=10.0 Hz, 1H). HRMS C₂₀H₁₇ClN₃O₂ (M+H)⁺ calculated mass, 366.1009; found, 366.1019.

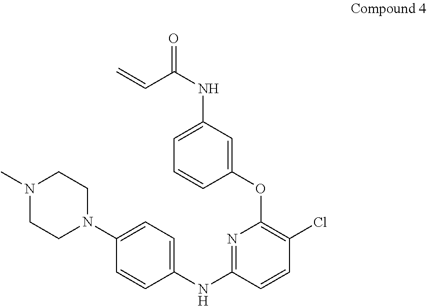 [SMILES: C=CC(=O)Nc1cccc(Oc2nc(Nc3ccc(N4CCN(C)CC4)cc3)ccc2Cl)c1]

### N-(3-((3-chloro-6-((4-(4-methylpiperazin-1-yl)phenyl)amino)pyridin-2-yl)oxy)phenyl)acrylamide

Prepared according to example 1 (scheme 1) using 4-(4-methylpiperazin-1-yl)aniline in step b to afford the title compound (8 mg) as a gray solid. ¹H NMR (400 MHz, MeOH) δ 7.82 (d, J=8.4 Hz, 1H), 7.53 (d, J=8.4 Hz, 1H), 7.41 (t, J=8.0 Hz, 1H), 7.30 (s, 1H), 7.16 (d, J=8.4 Hz, 2H), 6.91 (dd, J=8.0, 2.0 Hz, 1H), 6.42-6.38 (m, 3H), 5.78 (dd, J=9.6, 2.8 Hz, 1H), 3.59 (t, J=14.0 Hz, 4H), 3.27-3.20 (m, 2H), 2.96 (s, 3H), 2.92-2.87 (m, 2H). HRMS C₂₅H₂₇ClN₅O₂ (M+H)⁺ calculated mass, 464.1853; found, 464.1866.

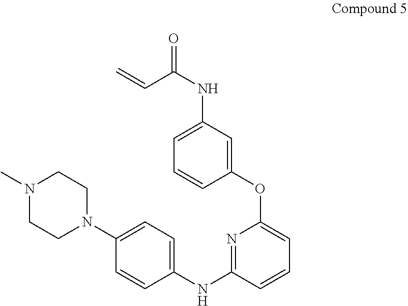 [SMILES: C=CC(=O)Nc1cccc(Oc2cccc(Nc3ccc(N4CCN(C)CC4)cc3)n2)c1]

### N-(3-((6-((4-(4-methylpiperazin-1-yl)phenyl)amino)pyridin-2-yl)oxy)phenyl)acrylamide

In purification of compound 4 by reverse-phase HPLC, we obtained compound 5 at the same time. ¹H NMR (400 MHz, MeOH) δ 7.67 (d, J=8.4 Hz, 1H), 7.49 (s, 1H), 7.40-7.36 (m, 2H), 7.27 (bs, 2H), 6.75 (bs, 2H), 6.89 (dd, J=8.0, 2.4 Hz, 1H), 6.46-6.33 (m, 3H), 6.24 (s, 1H), 5.77 (dd, J=9.6, 2.4 Hz, 1H), 6.42-6.38 (m, 3H), 5.78 (dd, J=9.6, 2.8 Hz, 1H), 3.64-3.56 (m, 4H), 3.27-3.20 (m, 2H), 2.96 (s, 3H), 2.92-2.88 (m, 2H). HRMS C₂₅H₂₈N₅O₂ (M+H)⁺ calculated mass, 430.2243; found, 430.2249.

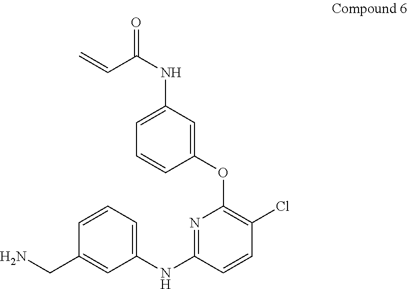 [SMILES: C=CC(=O)Nc1cccc(Oc2nc(Nc3cccc(CN)c3)ccc2Cl)c1]

### N-(3-((6-((3-(aminomethyl)phenyl)amino)-3-chloropyridin-2-yl)oxy)phenyl)acrylamide

Prepared according to example 1 (scheme 1) using tert-butyl (3-aminobenzyl)carbamate in step b and went through step c, d to obtain tert-butyl (3-((6-(3-acrylamidophenoxy)-5-chloropyridin-2-yl)amino)benzyl)carbamate (Intermediate 6d, MS m/z: 495.50 (M+1)). Intermediate 7d was dissolved in dichloromethane/TFA (2:1) and the mixture was stirred at room temperature for 2 hours. The mixture was evaporated to dryness, dissolved in methanol, and purified by reverse-phase HPLC eluting with acetonitrile containing 0.15‰ TFA to afford the title compound (13 mg) as a gray solid. ¹H NMR (400 MHz, DMSO-d₆) δ 10.28 (s, 1H), 9.39 (s, 1H), 8.03 (s, 2H), 7.76 (d, J=8.8 Hz, 1H), 7.58-7.57 (m, 1H), 7.55-7.54 (m, 1H), 7.43 (t, J=8.0 Hz, 1H), 7.30-7.27 (m, 1H), 7.20 (t, J=8.0 Hz, 1H), 7.00 (t, J=8.0 Hz, 1H), 6.97-6.94 (m, 1H), 6.87 (t, J=8.0 Hz, 1H), 6.60 (t, J=8.4 Hz, 1H), 6.40 (dd, J=16.8, 10.0 Hz, 1H), 6.24 (dd, J=17.2, 2.0 Hz, 1H), 5.76 (dd, J=10.0, 2.0 Hz, 1H), 3.74 (s, 2H). HRMS C₂₁H₂₀ClN₄O₂ (M+H)⁺ calculated mass, 395.1275; found, 395.1284.

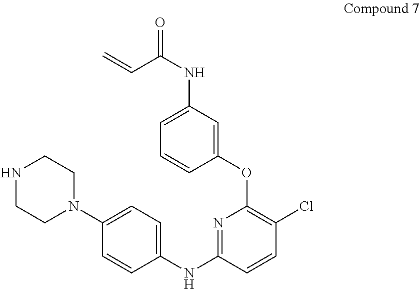 [SMILES: C=CC(=O)Nc1cccc(Oc2nc(Nc3ccc(N4CCNCC4)cc3)ccc2Cl)c1]

### N-(3-((3-chloro-6-((4-(piperazin-1-yl)phenyl)amino)pyridin-2-yl)oxy)phenyl)acrylamide

Prepared according to compound 6 (scheme 1) using tert-butyl 4-(4-aminophenyl)piperazine-1-carboxylate in step b to afford the title compound (10 mg) as a gray solid. ¹H NMR (400 MHz, DMSO-d₆) δ 10.34 (s, 1H), 9.11 (s, 1H), 8.76 (s, 1H), 7.67 (d, J=8.8 Hz, 1H), 7.65-7.63 (m, 1H), 7.49 (t, J=14 Hz, 1H), 7.41 (t, J=8.0 Hz, 1H), 7.13 (d, J=9.2 Hz, 2H), 6.91-6.88 (m, 1H), 6.63 (d, J=9.2 Hz, 2H), 6.47 (d, J=8.4 Hz, 1H), 6.43 (dd, J=16.8, 10.0 Hz, 1H), 6.26 (dd, J=16.8, 2.4 Hz, 1H), 5.77 (dd, J=10.0, 2.4 Hz, 1H), 3.21-3.18 (m, 4H), 3.15-3.12 (m, 4H). HRMS C₂₄H₂₅ClN₅O₂ (M+H)⁺ calculated mass, 450.1697; found, 450.1704.

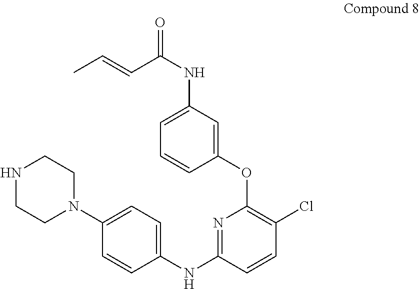 [SMILES: C/C=C/C(=O)Nc1cccc(Oc2nc(Nc3ccc(N4CCNCC4)cc3)ccc2Cl)c1]

### (E)-N-(3-((3-chloro-6-((4-piperazin-1-yl)phenyl)amino)pyridin-2-yl)oxy)phenyl)but-2-enamide

Prepared according to compound 7 (scheme 1) using (E)-but-2-enoyl chloride in step d to afford the title compound (9 mg) as a gray solid. ¹H NMR (400 MHz, DMSO-d₆ δ 10.13 (s, 1H), 7.61 (dd, J=8.0, 2.0 Hz, 1H), 7.48 (t, J=2.4 Hz, 1H), 7.37 (d, J=8.4 Hz, 1H), 7.13 (d, J=9.2 Hz, 2H), 6.79 (dd, J=15.2, 7.2 Hz, 1H), 6.63 (d, J=8.8 Hz, 2H), 6.43 (d, J=8.4 Hz, 1H), 6.11 (dd, J=15.2, 2.0 Hz, 1H), 5.98-5.88 (m, 1H), 5.19-5.09 (m, 1H), 3.13-3.12 (m, 4H), 3.11-3.10 (m, 4H), 1.86 (dd, J=6.8, 1.6 Hz, 1H). HRMS C₂₅H₂₇ClN₅O₂ (M+H)⁺ calculated mass, 464.1853; found, 464.1852.

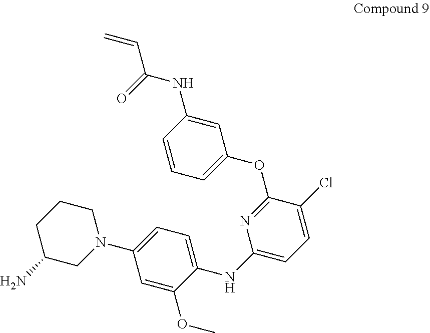 [SMILES: C=CC(=O)Nc1cccc(Oc2nc(Nc3ccc(N4CCC[C@@H](N)C4)cc3OC)ccc2Cl)c1]

### (R)-N-(3-((6-((4-(3-aminopiperidin-1-yl)-2-methoxyphenyl)amino)-3-chloropyridin-2-yl)oxy)phenyl)acrylamide

Prepared according to compound 6 (scheme 1) using tert-butyl (R)-(1-(4-amino-3-methoxyphenyl)piperidin-3-yl)carbamate in step b to afford the title compound (18 mg) as a gray solid. ¹H NMR (400 MHz, DMSO-d₆) δ 10.28 (s, 1H), 8.21 (s, 1H), 7.93 (bs, 2H), 7.64 (d, J=8.8 Hz, 1H), 7.61-7.59 (m, 1H), 7.46 (t, J=2.0 Hz, 1H), 7.36 (m, 2H), 6.88-6.85 (m, 1H), 5.62 (d, J=8.4 Hz, 1H), 6.53 (d, J=14 Hz, 1H), 6.42 (dd, J=16.8, 10.0 Hz, 1H), 6.26 (dd, J=17.2, 2.0 Hz, 1H), 6.08 (dd, J=8.8, 2.8 Hz, 1H), 5.77 (dd, J=10.0, 2.0 Hz, 1H), 3.76 (s, 3H), 3.35-3.32 (m, 2H), 3.13-3.09 (m, 1H), 2.90-2.81 (m, 2H), 1.89-1.80 (m, 2H), 1.61-1.52 (m, 2H). HRMS C₂₆H₂₉ClN₅O₃ (M+H)⁺ calculated mass, 494.1959; found, 494.1955.

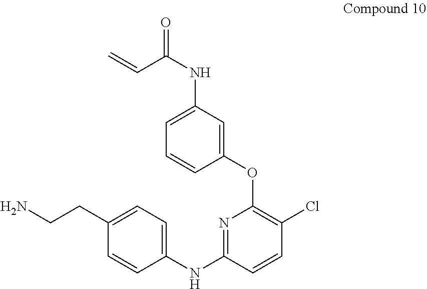 [SMILES: C=CC(=O)Nc1cccc(Oc2nc(Nc3ccc(CCN)cc3)ccc2Cl)c1]

### N-(3-((6-((4-(2-aminoethyl)phenyl)amino)-3-chloropyridin-2-yl)oxy)phenyl)acrylamide

Prepared according to compound 6 (scheme 1) using tert-butyl (4-aminophenethyl)carbamate in step b to afford the title compound (11 mg) as a gray solid. ¹H NMR (400 MHz, DMSO-d₆) δ 10.29 (s, 1H), 9.26 (s, 1H), 7.72 (d, J=8.8 Hz, 1H), 7.72 (s, 2H), 7.64-7.61 (m, 1H), 7.51 (s, 1H), 7.42 (t, J=8.4 Hz, 1H), 7.21-7.18 (m, 2H), 6.93-6.90 (m, 1H), 6.86-6.84 (m, 2H), 6.54 (d, J=8.4 Hz, 1H), 6.41 (dd, J=16.8, 10.0 Hz, 1H); 6.26 (dd, J=17.2, 2.0 Hz, 1H), 5.77 (dd, J=10.0, 2.0 Hz, 1H), 2.92 (bs, 2H), 2.67 (t, J=8.0 Hz, 2H). HRMS C₂₂H₂₂ClN₄O₂ (M+H)⁺ calculated mass, 409.1431; found, 409.1437.

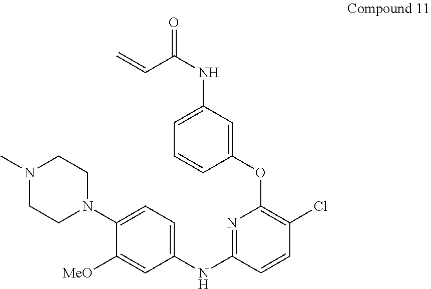 [SMILES: C=CC(=O)Nc1cccc(Oc2nc(Nc3ccc(N4CCN(C)CC4)c(OC)c3)ccc2Cl)c1]

### N-(3-((3-chloro-6-((3-methoxy-4-(4-methylpiperazin-1-yl)phenyl)amino)pyridin-2-yl)oxy)phenyl)acrylamide

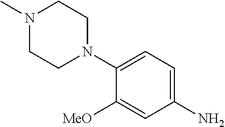 [SMILES: COc1cc(N)ccc1N1CCN(C)CC1]

3-methoxy-4-(4-methylpiperazin-1-yl)aniline was prepared following patent: WO2014/151871.

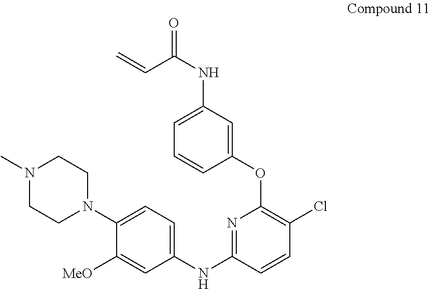 [SMILES: C=CC(=O)Nc1cccc(Oc2nc(Nc3ccc(N4CCN(C)CC4)c(OC)c3)ccc2Cl)c1]

Compound 11 was prepared according to example 1 (scheme 1) using 3-methoxy-4-(4-methylpiperazin-1-yl)aniline in step b to afford the title compound (13 mg) as a gray solid. ¹H NMR (400 MHz, DMSO-d₆) δ 10.27 (s, 1H), 9.53 (s, 1H), 9.22 (s, 1H), 7.72 (d, J=8.4 Hz, 1H), 7.59-7.56 (m, 2H), 7.45 (d, J=2.4 Hz, 1H), 7.04 (d, J=2.4 Hz, 1H), 6.88-6.85 (m, 1H), 6.80 (dd, J=8.4, 2.4 Hz, 1H), 6.58 (d, J=8.4 Hz, 1H), 6.56 (d, J=8.4 Hz, 1H), 6.40 (dd, J=17.2, 10.0 Hz, 1H), 6.25 (dd, J=17.2, 2.0 Hz, 1H), 5.76 (dd, J=10.0, 2.4 Hz, 1H), 3.44 (d, J=12.0 Hz, 2H), 3.40 (s, 3H), 3.30 (d, J=12.8 Hz, 2H), 3.18-3.10 (m, 2H), 2.84-2.73 (m, 5H). HRMS C₂₆H₂₉ClN₅O₃ (M+H)⁺ calculated mass, 494.1959; found, 494.1967.

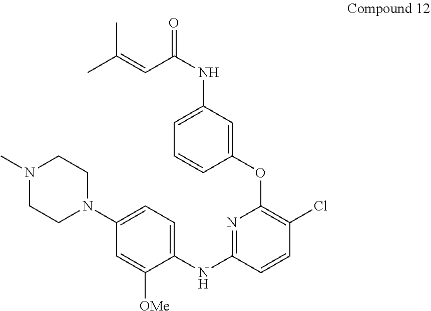 [SMILES: COc1cc(N2CCN(C)CC2)ccc1Nc1ccc(Cl)c(Oc2cccc(NC(=O)C=C(C)C)c2)n1]

### N-(3-((3-chloro-6-((2-methoxy-4-(4-methylpiperazin-1-yl)phenyl)amino)pyridin-2-yl)oxy)phenyl)-3-methylbut-2-enamide

Prepared according to example 1 (scheme 1) using 3-methylbut-2-enoyl chloride in step d to afford the title compound (16 mg) as a gray solid. ¹H NMR (400 MHz, DMSO-d₆) δ 9.96 (s, 1H), 8.13 (s, 1H), 7.61 (d, J=8.4 Hz, 1H), 7.53-7.49 (m, 1H), 7.43 (t, J=2.4 Hz, 1H), 7.34 (t, J=8.4 Hz, 1H), 7.30 (d, J=8.8 Hz, 1H), 6.79-6.77 (m, 1H), 6.59 (d, J=8.4 Hz, 1H), 6.52 (d, J=2.4 Hz, 1H), 5.85 (t, J=1.6 Hz, 1H), 3.78 (s, 3H), 3.03 (bs, 4H), 2.28 (bs, 4H), 2.13 (s, 3H), 1.86-1.84 (m, 3H), 1.74 (s, 3H). HRMS C₂₈H₃₃ClN₅O₃(M+H)⁺ calculated mass, 522.2272; found, 522.2275.

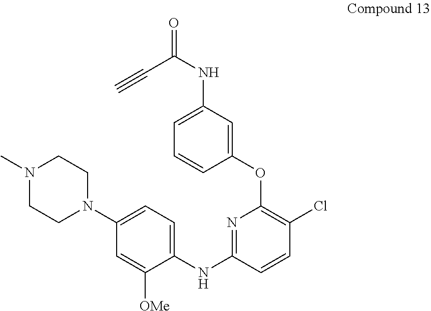 [SMILES: C#CC(=O)Nc1cccc(Oc2nc(Nc3ccc(N4CCN(C)CC4)cc3OC)ccc2Cl)c1]

### N-(3-((3-chloro-6-((2-methoxy-4-(4-methylpiperazin-1-yl)phenyl)amino)pyridin-2-yl)oxy)phenyl)propiolamide

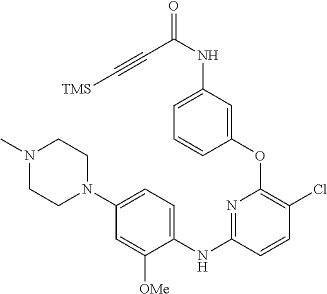 [SMILES: COc1cc(N2CCN(C)CC2)ccc1Nc1ccc(Cl)c(Oc2cccc(NC(=O)C#C[Si](C)(C)C)c2)n1]

### N-(3-((3-chloro-6-((2-methoxy-4-(4-methylpiperazin-1-yl)phenyl)amino)pyridin-2-yl)oxy)phenyl)-3-(trimethylsilyl)propiolamide

3-(trimethylsilyl)propiolic acid (1.1 eq) was dissolved in anhydrous dichloromethane, oxalyl chloride (1.44 eq) was added dropwise at 0° C. and then one drop DMF was added. The reaction mixture stirred at room temperature for 2 hours. The excess oxalyl chloride was removed under reduced pressure, and the residue dissolved in dichloromethane for next transformation. Intermediate 2c (1 eq) and triethylamine (1.5 eq) were added sequently to a 25 ml three-neck-bottom flask under a nitrogen atmosphere. 3-(trimethylsilyl)propioloyl chloride dichloromethane was added dropwise to flask at 0° C. The reaction mixture was stirred at 0° C. for 1 hour. Saturated sodium bicarbonate solution was added to reaction mixture at 0° C., and the aqueous phase was extracted with dichloromethane twice. The combined organic phase was washed with saturated brine, then dried over anhydrous sodium sulfate. The organic phase was filtered off and then concentrated under reduced pressure. LC-MS m/z: 564.73 (M+1)

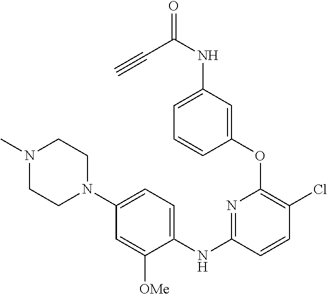 [SMILES: C#CC(=O)Nc1cccc(Oc2nc(Nc3ccc(N4CCN(C)CC4)cc3OC)ccc2Cl)c1]

To a solution of intermediate N-(3-((3-chloro-6-((2-methoxy-4-(4-methylpiperazin-1-yl)phenyl)amino)pyridin-2-yl)oxy)phenyl)-3-(trimethylsilyl)propiolamide in THF was added a 1M solution of TBAF in THF (10 eq). The mixture was stirred at room temperature for 2 hours. Water and ethyl acetate were added to the reaction solution, separated and then the aqueous phase was extracted with ethyl acetate. The combined organic phase was washed with saturated brine, then dried over anhydrous sodium sulfate. The organic phase was filtered off and then concentrated under reduced pressure. The obtained residue was dissolved in dichloromethane (2 ml) and TFA (0.25 ml) to afford the TFA salt of title compound. Dichloromethane and excess TFA was removed in vacuo and the residue dissolved in methanol and purified by reverse-phase HPLC eluting with acetonitrile containing 0.15‰ TFA to afford the title compound (10 mg) as a gray solid. NMR (400 MHz, DMSO-d₆) δ 10.94 (s, 1H), 9.62 (s, 1H), 8.23 (s, 1H), 7.64 (d, J=8.8 Hz, 1H), 7.58-7.55 (m, 1H); 7.38 (t, J=8.4 Hz, 1H), 7.36-7.32 (m, 1H), 6.91-6.88 (m, 1H), 6.64 (d, J=8.4 Hz, 1H), 6.60 (d, J=2.4 Hz, 1H), 6.08 (dd, J=8.8, 2.4 Hz, 1H), 4.45 (s, 1H), 3.78 (s, 3H), 3.69 (bs, 2H), 3.49 (bs, 2H), 3.12 (bs, 2H), 2.87 (bs, 2H), 2.86 (s, 3H). HRMS C₂₆H₂₇ClN₅O₃ (M+H)⁺ calculated mass, 492.1802; found, 492.1801.

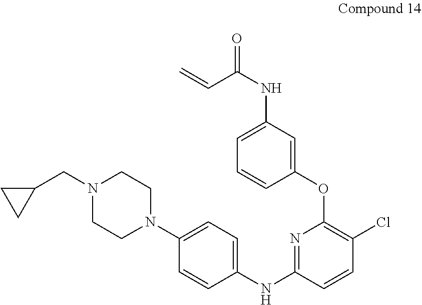 [SMILES: C=CC(=O)Nc1cccc(Oc2nc(Nc3ccc(N4CCN(CC5CC5)CC4)cc3)ccc2Cl)c1]

### N-(3-((3-chloro-6-((4-(4-(cyclopropylmethyl)piperazin-1-yl)phenyl)amino)pyridin-2-yl)oxy)phenyl)acrylamide

Compound 7 (30 mg, 0.067 mmol) was dissolved in DMF (2 ml), then potassium carbonate (18 mg, 0.13 mmol) and (bromomethyl)cyclopropane (9 mg, 0.067 mmol) were added. The mixture was stirred at 70° C. for 4 hours. Water and ethyl acetate were added to the reaction mixture, separated and then the aqueous phase was extracted with ethyl acetate three times. The combined organic phase was washed with saturated brine, then dried over anhydrous sodium sulfate. The organic phase was filtered off and then concentrated under reduced pressure. The obtained residue was dissolved in dichloromethane (2 ml) and TFA (0.25 ml) to afford the TFA salt of title compound. Dichloromethane and excess TFA was removed in vacuo and the residue dissolved in methanol and purified by reverse-phase HPLC eluting with acetonitrile containing 0.15‰ TFA to afford the title compound (5 mg) as a gray solid. NMR (400 MHz, DMSO-4) δ 10.32 (s, 1H), 9.55 (s, 1H), 9.09 (s, 1H), 7.68-7.64 (m, 2H), 7.47 (t, J=2.0 Hz, 1H), 7.41 (t, J=8.0 Hz, 1H), 7.14 (d, J=9.2 Hz, 2H), 6.91-6.88 (m, 1H), 6.65 (d, J=9.2 Hz, 2H), 6.47 (d, J=8.8 Hz, 2H), 6.42 (dd, J=17.2, 10.0 Hz, 1H), 6.26 (dd, J=17.2, 2.0 Hz, 1H), 5.77 (dd, J=10.0, 2.0 Hz, 1H), 3.60 (d, J=11.6 Hz, 2H), 3.15-3.06 (m, 4H), 2.86 (t, J=12.0 Hz, 2H), 1.12-1.06 (m, 1H), 0.70-0.65 (m, 2H), 0.41-0.37 (m, 2H). HRMS C₂₈H₃₁ClN₅O₂(M+H)⁺ calculated mass, 504.2166; found, 504.2181.

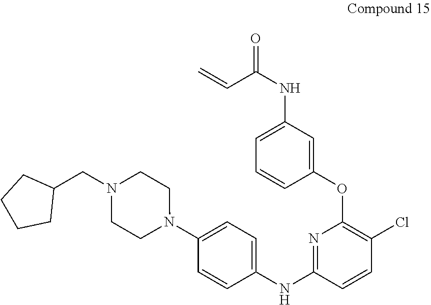 [SMILES: C=CC(=O)Nc1cccc(Oc2nc(Nc3ccc(N4CCN(CC5CCCC5)CC4)cc3)ccc2Cl)c1]

### N-(3-((3-chloro-6-((4-(4-(cyclopentylmethyl)piperazin-1-yl)phenyl)amino)pyridin-2-yl)oxy)phenyl)acrylamide

Prepared according to compound 14 using (bromomethyl)cyclopentane in last step to afford the title compound (7 mg) as a gray solid. ¹H NMR (400 MHz, DMSO-d₆) δ 10.32 (s, 1H), 9.09 (s, 1H), 7.68-7.64 (m, 2H), 7.47 (s, 1H), 7.41 (t, J=8.0 Hz, 1H), 7.14 (d, J=8.8 Hz, 1H), 7.54-7.51 (m, 2H), 6.91-6.88 (m, 1H), 6.64 (d, J=8.8 Hz, 1H), 6.47 (d, J=8.8 Hz, 1H), 6.43 (dd, J=16.8, 10.0 Hz, 1H), 6.26 (dd, J=16.8, 2.0 Hz, 1H), 5.77 (dd, J=10.0, 2.0 Hz, 1H), 3.57-3.54 (m, 2H), 3.38-3.28 (m, 2H), 3.17-3.14 (m, 2H), 2.94-2.88 (m, 2H), 1.87-1.81 (m, 2H), 1.65-1.52 (m, 4H), 1.28-1.21 (m, 5H). HRMS C₃₀H₃₅ClN₅O₂ (M+H)⁺ calculated mass, 532.2479; found, 532.2522.

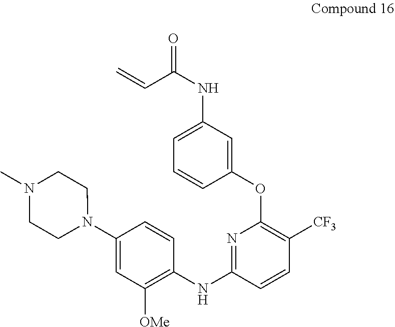 [SMILES: C=CC(=O)Nc1cccc(Oc2nc(Nc3ccc(N4CCN(C)CC4)cc3OC)ccc2C(F)(F)F)c1]

### N-(3-((6-((2-methoxy-4-(4-methylpiperazin-1-yl)phenyl)amino)-3-(trifluoromethyl)pyridin-2-yl)oxy)phenyl)acrylamide

Prepared according to example 1 (scheme 1) using 2,6-dichloro-3-(trifluoromethyl)pyridine in step a to afford the title compound (20 mg) as a gray solid. ¹H NMR (400 MHz, DMSO-d₆) δ 10.32 (s, 1H), 7.96 (d, J=8.4 Hz, 1H), 7.66-7.64 (m, 1H), 7.55 (t, J=2.4 Hz, 1H), 7.46 (d, J=2.0 Hz, 1H), 7.42 (t, J 8.0 Hz, 1H), 7.33 (d, J=8.8 Hz, 1H), 6.92-6.89 (m, 1H), 6.55 (d, J=2.4 Hz, 1H), 6.46 (d, J=8.8 Hz, 1H), 6.41 (dd, =16.8, 10.0 Hz, 1H), 6.25 (dd, J=16.8, 2.0 Hz, 1H), 6.00 (dd, =8.8, 2.4 Hz, 1H), 5.77 (dd, J=10.0, 2.0 Hz, 1H), 3.80 (s, 3H), 3.01 (t, J=4.8 Hz, 4H), 2.42 (t, J=4.8 Hz, 4H), 2.21 (s, 3H). HRMS C₂₇H₂₉F₃N₅O₃ (M+H)⁺ calculated mass, 528.2222; found, 528.2232.

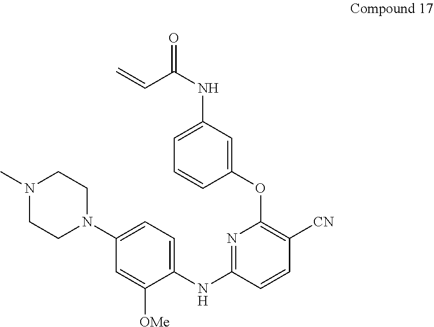 [SMILES: C=CC(=O)Nc1cccc(Oc2nc(Nc3ccc(N4CCN(C)CC4)cc3OC)ccc2C#N)c1]

### N-(3-((3-cyano-6-((2-methoxy-4-(4-methylpiperazin-1-yl)phenyl)amino)pyridin-2-yl)oxy)phenyl)acrylamide

Prepared according to example 1 (scheme 1) using 2,6-dichloronicotinonitrile in step a to afford the title compound (19 mg) as a gray solid. NMR (400 MHz, DMSO-d₆) δ 10.28 (s, 1H), 8.31 (s, 1H), 7.59-7.57 (m, 1H), 7.57 (d, J=8.4 Hz, 1H), 7.50 (t, J=2.4 Hz, 1H), 7.37 (t, J=8.0 Hz, 1H), 7.29 (d, J=9.2 Hz, 1H), 6.85-6.82 (m, 1H), 6.54 (d, J=8.8 Hz, 1H), 6.51 (d, J=2.4 Hz, 1H), 6.42 (dd, J=16.8, 10.0 Hz, 1H), 6.25 (dd, J=16.8, 2.0 Hz, 1H), 6.02 (dd, =8.8, 2.4 Hz, 1H), 5.76 (dd, J=10.0, 2.0 Hz, 1H), 3.75 (s, 3H), 3.00 (t, J=4.8 Hz, 4H), 2.41 (t, J=4.8 Hz, 4H), 2.21 (s, 3H). HRMS C₂₇H₂₉N₆O₃ (M+H)⁺ calculated mass, 485.2301; found, 485.2312.

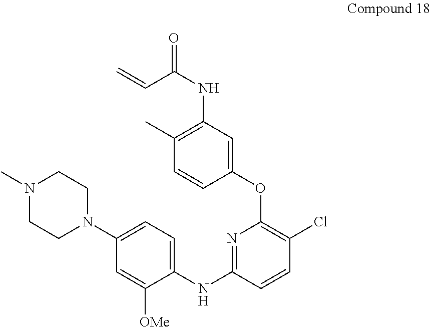 [SMILES: C=CC(=O)Nc1cc(Oc2nc(Nc3ccc(N4CCN(C)CC4)cc3OC)ccc2Cl)ccc1C]

### N-(5-((3-chloro-6-((2-methoxy-4-(4-methylpiperazin-1-yl)phenyl)amino)pyridin-2-yl)oxy)-2-methylphenyl)acrylamide

Prepared according to example 1 (scheme 1) using 4-methyl-3-nitrophenol in step a to afford the title compound (16 mg) as a gray solid. ¹H NMR (400 MHz, DMSO-d₆) δ 9.52 (s, 1H), 8.18 (s, 1H), 7.62 (d, J=8.4 Hz, 1H), 7.39 (s, 1H), 7.34 (d, J=8.8 Hz, 1H), 7.27 (d, J=8.4 Hz, 1H), 6.88 (dd, =7=8.0, 2.4 Hz, 1H), 6.60-6.58 (m, 2H), 6.57 (dd, J==16.8, 10.0 Hz, 1H), 6.23 (dd, J=17.2, 2.0 Hz, 1H), 6.11 (dd, 8.8, 2.8 Hz, 1H), 5.75 (dd, J=10.0, 2.0 Hz, 1H), 3.77 (s, 3H), 3.71 (bs, 2H), 3.49 (its, 2H), 3.14 (bs, 2H), 2.86 (Its, 5H), 2.27 (s, 3H). HRMS C₂₁H₃₁ClN₅O₃ (M+H)⁺ calculated mass, 508.2115; found, 508.2112.

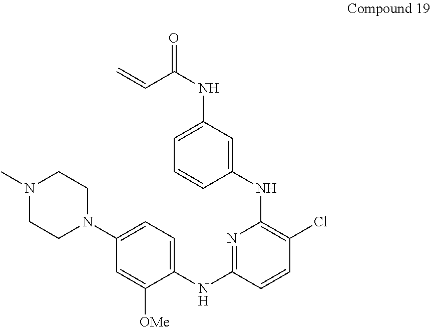 [SMILES: C=CC(=O)Nc1cccc(Nc2nc(Nc3ccc(N4CCN(C)CC4)cc3OC)ccc2Cl)c1]

### N-(3-((3-chloro-6-((2-methoxy-4-(4-methylpiperazin-1-yl)phenyl)amino)pyridin-2-yl)amino)phenyl)acrylamide

### 3,6-dichloro-N-(3-nitrophenyl)pyridin-2-amine

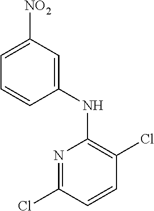 [SMILES: O=[N+]([O-])c1cccc(Nc2nc(Cl)ccc2Cl)c1]

3-nitroaniline (763 mg, 5.53 mmol) was dissolved in anhydrous DMF, NaH (60%, 332.7 mg, 8.29 mmol) was added at 0° C., the reaction mixture was stirred at 0° C. for 1 hour, then 2,3,6-trichloropyridine (1.0 g, 5.53 mmol) was added. The reaction mixture was heated to 45′C for 4 h. The reaction mixture was diluted with water at 0° C., stirred for 10 min and extracted with ethyl acetate three times, the combined organic phase was washed with brine. The organic phase was dried over anhydrous sodium sulfate and concentrated in vacuo, the crude product was purified by silica gel chromatograph to afford 782 mg of yellow solid (yield: 50%). LC-MS m/z: 283.94 (M+1).

### N-(3-((3-chloro-6-((2-methoxy-4-(4-methylpiperazin-1-yl)phenyl)amino)pyridin-2-yl)amino)phenyl)acrylamide

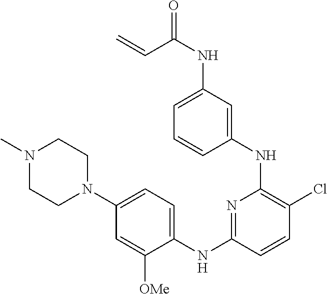 [SMILES: C=CC(=O)Nc1cccc(Nc2nc(Nc3ccc(N4CCN(C)CC4)cc3OC)ccc2Cl)c1]

Prepared according to example 1 using 3,6-dichloro-N-(3-nitrophenyl)pyridin-2-amine in step b to afford the title compound (12 mg) as a gray solid. ¹H NMR (400 MHz, DMSO-d₆) δ 10.02 (s, 1H), 8.00 (s, 1H), 7.78 (t, J=2.0 Hz, 1H), 7.74 (s, 1H), 7.61 (d, J=8.8 Hz, 1H), 7.40 (t, J=8.4 Hz, 1H), 7.37-7.35 (m, 1H), 7.28-7.24 (m, 1H), 7.15 (t, J=8.0 Hz, 1H), 6.57 (d, J=2.4 Hz, 1H), 6.43 (dd, J=16.8, 10.0 Hz, 1H), 6.26-6.24 (m, 2H), 6.22 (dd, J=4.0, 2.4 Hz, 1H), 5.73 (dd, J=10.0, 2.0 Hz, 1H), 3.77 (s, 3H), 3.04 (t, J=5.2 Hz, 4H), 2.43 (t, J=5.2 Hz, 4H), 2.22 (s, 3H). HRMS C₂₆H₃₀ClN₆O₂ (M+H)⁺ calculated mass, 493.2119; found, 493.2113.

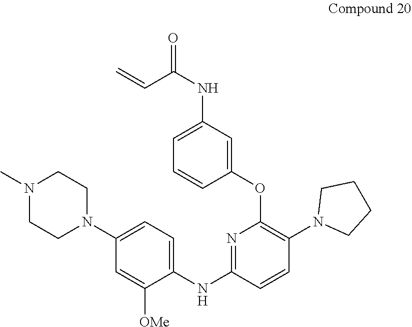 [SMILES: C=CC(=O)Nc1cccc(Oc2nc(Nc3ccc(N4CCN(C)CC4)cc3OC)ccc2N2CCCC2)c1]

### N-(3-((6-((2-methoxy-4-(4-methylpiperazin-1-yl)phenyl)amino)-3-(pyrrolidin-1-yl)pyridin-2-yl)oxy)phenyl)acrylamide

Prepared according to example 2 (scheme 2) using 3-nitrophenol in step a and pyrrolidine in step b to afford the title compound (14 mg) as a gray solid. ¹H NMR (400 MHz, DMSO-d₆) δ 10.30 (s, 1H), 9.69 (s, 1H), 7.57-7.53 (m, 1H), 7.40-7.35 (m, 2H), 6.89-6.85 (m, 1H), 6.64-6.59 (m, 2H), 6.07-6.00 (m, 1H), 6.42 (dd, J=16.8, 10.0 Hz, 1H), 6.25 (dd J=16.8, 2.0 Hz, 1H), 5.77 (dd, J=10.0, 2.0 Hz, 1H), 3.79 (s, 3H), 3.50 (d, J=12.0 Hz, 4H), 3.12 (bs, 2H), 2.87-2.77 (m, 5H), 1.99 (bs, 4H). HRMS C₃₀H₃₇N₆O₃ (M+H)⁺ calculated mass, 529.2927; found, 529.2923.

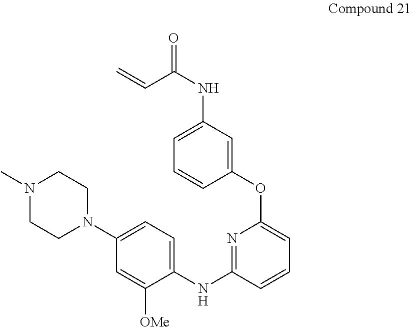 [SMILES: C=CC(=O)Nc1cccc(Oc2cccc(Nc3ccc(N4CCN(C)CC4)cc3OC)n2)c1]

### N-(3-((6-((2-methoxy-4-(4-methylpiperazin-1-yl)phenyl)amino)pyridin-2-yl)oxy)phenyl)acrylamide

In purification of compound 20 by reverse-phase HPLC, ore obtained compound 21 at the same time. ¹H NMR (400 MHz, DMSO-d₆) δ 10.27 (s, 1H), 8.00 (s, 1H), 7.57-7.49 (m, 3H), 7.46-7.44 (m, 1H), 7.36 (t, J=8.0 Hz, 1H), 6.84-6.81 (m, 1H), 6.62 (d, J=2.81 Hz, 1H), 6.56 (d, J=8.0 Hz, 1H), 6.42 (dd, J=16.8, 10.0 Hz, 1H), 6.25 (dd, J=16.8, 2.0 Hz, 1H), 6.21 (d, J=8.0 Hz, 1H), 6.18 (dd, J=8.8, 2.4 Hz, 1H), 5.76 (dd, J=10.0, 2.0 Hz, 1H), 3.79 (s, 3H), 3.70 (d, J=13.2 Hz, 2H), 3.50 (d, J=12.0 Hz, 2H), 3.18-3.09 (m, 2H), 2.87-2.82 (m, 5H). HRMS C₂₆H₃₀N₅O₃ (M+H)⁺ calculated mass, 460.2349; found, 460.2361.

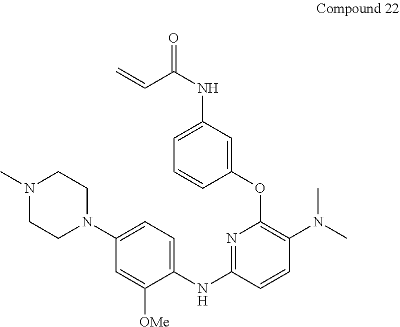

### N-(3-((3-(dimethylamino)-6-((2-methoxy-4-(4-methylpiperazin-1-yl)phenyl)amino)pyridin-2-yl)ox)phenyl)acrylamide

Prepared according to example 2 (scheme 2) using 3-nitrophenol in step a and dimethylamine in step b to afford the title compound (20 mg) as a gray solid. ¹H NMR (400 MHz, DMSO-d₆) δ: 10.24 (s, 7.57-7.54 (m, 1H), 7.50 (s, 1H), 7.46 (t, J=2.4 Hz, 1H), 7.37-7.33 (m, 2H), 7.28 (d, J=8.4 Hz, 1H), 6.82-6.79 (m, 1H), 6.56 (d, J=8.4 Hz, 1H), 6.50 (d, J=2.8 Hz, 1H), 6.41 (dd, J=16.8, 10.0 Hz, 1H), 6.24 (dd, J=16.8, 2.0 Hz, 1H), 6.02 (dd, J=8.8, 2.4 Hz, 1H), 6.75 (dd, J=10.0, 2.0 Hz, 1H), 3.75 (s, 3H), 2.97 (t, J=5.2 Hz, 1H), 2.69 (s, 6H), 2.43 (t, J=5.2 Hz, 1H), 2.22 (s, 3H). HRMS C₂₈H₃₅N₆O₃ (M+H)⁺ calculated mass, 503.2771; found, 503.2770.

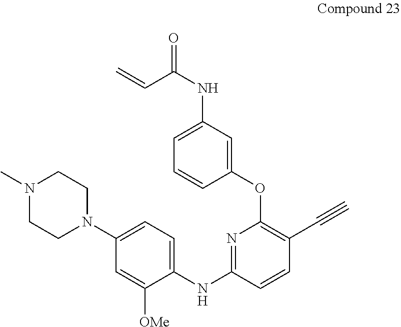 [SMILES: C#Cc1ccc(Nc2ccc(N3CCN(C)CC3)cc2OC)nc1Oc1cccc(NC(=O)C=C)c1]

### N-(3-((3-ethynyl-6-((2-methoxy-4-(4-methylpiperazin-1-yl)phenyl)amino)pyridin-2-yl)oxy)phenyl)acrylamide

### 3-((3-bromo-6-chloropyridin-2-yl)oxy)aniline

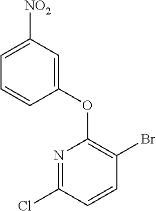 [SMILES: O=[N+]([O-])c1cccc(Oc2nc(Cl)ccc2Br)c1]

Prepared according to intermediate 2a (scheme 2) using 3-nitro enol in step a to afford the title compound as a white solid, LC-MS m/z: 328.94 (M+1).

### 3-((6-chloro-3-((trimethylsilyl)ethynyl)pyridin-2-yl)oxy)aniline

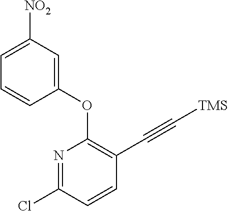 [SMILES: C[Si](C)(C)C#Cc1ccc(Cl)nc1Oc1cccc([N+](=O)[O-])c1]

A flask containing 3-bromo-6-chloro-2-(3-nitrophenoxy)pyridine (275 mg, 0.83 mmol), Pd(PPh₃)₂Cl₂ (59 mg, 0.083 mmol) and CuI (16 mg, 0.083 mmol) was evacuated and back filled with nitrogen three times. Then was added sequentially, with stirring, dioxane (4 ml), triethylamine (126 mg, 1.25 mmol) and ethynyltrimethylsilane (98 mg, 1.00 mmol). The resulting solution was stirred at 70° C. for 4 hours, before quenching with sat. ammonium chloride (aq). The mixture was diluted with ethyl acetate, and washed with brine. The organic layers were dried over anhydrous sodium sulfate and concentrated in vacuo to afford the crude, which was purified by silica gel chromatography to afford the title product. LC-MS m/z: 347.12 (M+1).

### 6-(3-aminophenoxy)-N-(2-methoxy-4-(4-methylpiperazin-1-yl)phenyl)-5-((trimethylsilyl)ethynyl)pyridin-2-amine

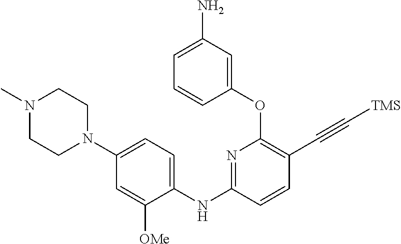 [SMILES: COc1cc(N2CCN(C)CC2)ccc1Nc1ccc(C#C[Si](C)(C)C)c(Oc2cccc(N)c2)n1]

Prepared according to the methods of step b and step c mentioned in scheme 1 to afford the title compound as a brown solid. LC-MS m/z: 502.28 (M+1).

### 6-(3-aminophenoxy)-5-ethynyl-N-(2-methoxy-4-(4-methylpiperazin-1-yl)phenyl)pyridin-2-amine

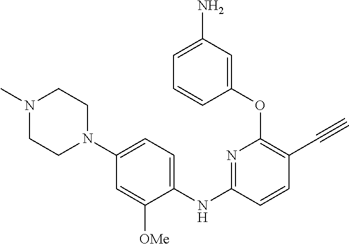 [SMILES: C#Cc1ccc(Nc2ccc(N3CCN(C)CC3)cc2OC)nc1Oc1cccc(N)c1]

Prepared according to the method mentioned in preparation of compound 13 to afford the title compound as a brown solid. LC-MS m/z: 430.20 (M+1).

### N-(3-((3-ethynyl-6-((2-methoxy-4-(4-methylpiperazin-1-yl)phenyl)amino)pyridin-2-yl)oxy)phenyl)acrylamide

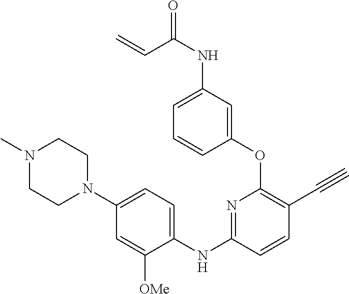 [SMILES: C#Cc1ccc(Nc2ccc(N3CCN(C)CC3)cc2OC)nc1Oc1cccc(NC(=O)C=C)c1]

Prepared according to the method of step e in example 2 (scheme 2) to afford the title compound as a gray solid. ¹H NMR (400 MHz, DMSO-d₆) δ 10.32 (s, 1H), 8.04 (d, J=8.4 Hz, 1H), 7.89 (s, 1H), 7.61-7.58 (m, 1H), 7.56 (t, J=2.0 Hz, 1H), 7.40 (t, J=8.4 Hz, 1H), 7.26 (d, J=8.8 Hz, 1H), 6.90-6.87 (m, 1H) 6.55 (d, J=2.8 Hz, 1H), 6.42 (d, J=8.4 Hz, 1H), 6.42 (dd, J=16.8, 10.0 Hz, 1H), 6.25 (dd, J=16.8, 2.0 Hz, 1H), 6.07 (dd, J=8.8, 2.4 Hz, 1H), 5.77 (dd, J=10.0, 2.0 Hz, 1H), 3.79 (s, 3H), 3.03 (t, J=4.8 Hz, 4H), 2.42 (t, J=4.8 Hz, 4H), 2.22 (s, 3H). HRMS C₂₈H₃₀N₅O₃ (M+H)⁺ calculated mass, 484.2349; found, 484.2358.

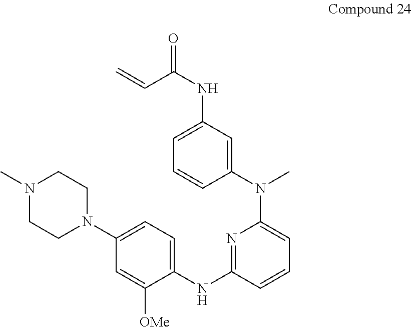

### N-(3-((6-((2-methoxy-4-(4-methylpiperazin-1-yl)phenyl)amino)pyridin-2-yl)(methyl)amino)phenyl)acrylamide

### 6-chloro-N-(3-nitrophenyl)pyridin-2-amine

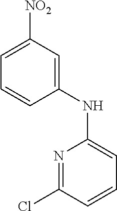 [SMILES: O=[N+]([O-])c1cccc(Nc2cccc(Cl)n2)c1]

Prepared according to the method of step b in scheme 1 using 2,6-dichloropyridine and 3-nitroaniline as the reagents. LC-MS m/z: 250.17 (M+1).

### 6-chloro-N-methyl-N-(3-nitrophenyl)pyridin-2-amine

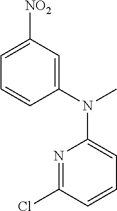 [SMILES: CN(c1cccc([N+](=O)[O-])c1)c1cccc(Cl)n1]

6-chloro-N-(3-nitrophenyl)pyridine-2-amine (160 mg, 0.56 mmol) was dissolved in anhydrous DMF (2 ml), NaH (60%, 34 mg, 0.85 mmol) was added at 0° C., the reaction mixture was stirred at 0° C. for 1 hour, then iodomethane (88 mg, 0.62 mmol) was added. The reaction mixture was stirred at 0° C. for 20 min. The reaction mixture was diluted with water at 0° C., stirred for 10 min and extracted with ethyl acetate three times, the combined organic phase was washed with brine. The organic phase wad dried over anhydrous sodium sulfate and concentrated, the crude product was purified by silica gel chromatograph to afford 138 mg of yellow solid (yield: 82%), LC-MS m/z: 264.26 (M+1).

### N-(3-((6-((2-methoxy-4-(4-methylpiperazin-1-yl)phenyl)amino)pyridin-2-yl)(methyl)amino)phenyl)acrylamide

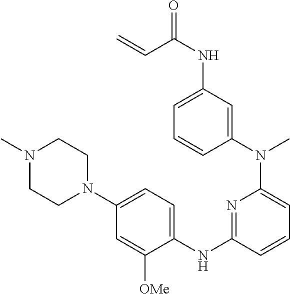

Prepared according to example 1 using 6-chloro-N-methyl-N-(3-nitrophenyl)pyridin-2-amine in step h to afford the title compound (18 mg) as a gray solid. NMR (400 MHz, DMSO-d₆) δ 10.18 (s, 1H), 7.77 (d, J=8.8 Hz, 1H), 7.61 (t, J=2.0 Hz, 1H), 7.54-7.51 (m, 1H), 7.48 (s, 1H), 7.34 (t, J=8.0 Hz, 1H), 7.19 (t, J=8.0 Hz, 1H), 6.98-6.96 (m, 1H), 6.58 (d, J=2.4 Hz, 1H), 6.41 (dd, J=16.8, 10.0 Hz, 1H), 6.33 (dd, =8.8, 2.4 Hz, 1H), 6.24 (dd, J=16.8, 2.0 Hz, 1H), 6.16 (d, J=8.0 Hz, 1H), 5.90 (d, J=8.0 Hz, 1H), 5.75 (dd, J=10.0, 2.0 Hz, 1H), 3.80 (s, 3H), 3.34 (s, 3H), 3.06 (t, J=4.8 Hz, 4H), 2.44 (J=4.8 Hz, 4H), 2.22 (s, 3H). HRMS C₂₇H₃₃N₆O₂ (M+H)⁺ calculated mass, 473.2665; found, 473.2676.

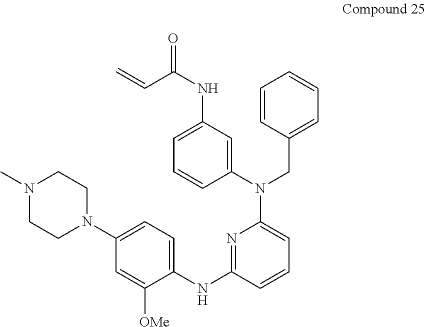 [SMILES: C=CC(=O)Nc1cccc(N(Cc2ccccc2)c2cccc(Nc3ccc(N4CCN(C)CC4)cc3OC)n2)c1]

### N-(3-(benzyl(6-((2-methoxy-4-(4-methylpiperazin-1-yl)phenyl)amino)pyridin-2-yl)amino)phenyl)acrylamide

Prepared according to compound 24 using (bromomethyl)benzene instead of iodomethane to afford the product (16 mg) as a gray solid. ¹H NMR (400 MHz, DMSO-d₆) δ 10.14 (s, 1H), 7.63 (d, J=2.0 Hz, 1H), 7.53-7.48 (m, 3H), 7.31-7.25 (m, 4H), 7.21-7.17 (m, 2H), 6.98-6.95 (m, 1H), 6.56 (d, J=2.4 Hz, 1H), 6.39 (dd, J=16.8, 9.6 Hz, 1H), 6.22 (dd, J=16.8, 2.0 Hz, 1H), 6.17 (d, J=8.0 Hz, 1H), 6.14 (dd, J=8.8, 2.4 Hz, 1H), 5.93 (d, J=8.0 Hz, 1H), 5.73 (dd, J=10.0, 2.0 Hz, 1H), 5.14 (s, 2H), 3.78 (s, 3H), 3.04 (bs, 4H), 2.48 (bs, 4H), 2.25 (s, 3H). HRMS C₃₃H₃₇N₆O₂(M+H)⁺ calculated mass, 549.2978; found, 549.2986.

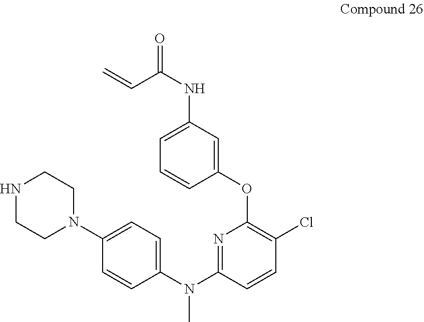 [SMILES: C=CC(=O)Nc1cccc(Oc2nc(N(C)c3ccc(N4CCNCC4)cc3)ccc2Cl)c1]

### N-(3-((3-chloro-6-(methyl(4-(4-methylpiperazin-1-yl)phenyl)amino)pyridin-2-yl)oxy)phenyl)acrylamide

### tert-butyl 4-(4-((5-chloro-6-(3-nitrophenoxy)pyridin-2-yl)amino)phenyl)piperazine-1-carboxylate

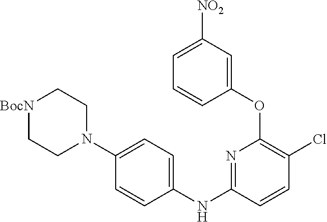 [SMILES: CC(C)(C)OC(=O)N1CCN(c2ccc(Nc3ccc(Cl)c(Oc4cccc([N+](=O)[O-])c4)n3)cc2)CC1]

Prepared according to compound 7 through step a, b to afford the product as a gray solid. LC-MS m/z: 526.55 (M+1).

### tert-butyl 4-(4-((5-chloro-6-(3-nitrophenoxy)pyridin-2-yl)(methyl)amino)phenyl)piperazine-1-carboxylate

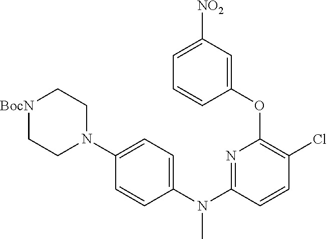 [SMILES: CN(c1ccc(N2CCN(C(=O)OC(C)(C)C)CC2)cc1)c1ccc(Cl)c(Oc2cccc([N+](=O)[O-])c2)n1]

tert-butyl 4-(4-((5-chloro-6-(3-nitrophenoxy)pyridin-2-yl)(methyl)amino)phenyl)piperazine-1-carboxylate (85 mg, 0.16 mmol) was dissolved in anhydrous DMF (1.5 ml), NaH (60%, 6 mg, 0.24 mmol) was added at 0° C., the reaction mixture was stirred at 0° C. for 20 min, then iodomethane (25 mg, 0.17 mmol) was added. The reaction mixture was stirred at 0° C. for 15 min. The reaction mixture was diluted with water at 0° C., stirred for 10 min and extracted with ethyl acetate three times, the combined organic phase was washed with brine. The organic phase wad dried over anhydrous sodium sulfate and concentrated, the residue product was purified by silica gel chromatograph to afford 66 mg of yellow solid (yield: 76%). LC-MS m/z: 540.49 (M+1).

### N-(3-((3-chloro-6-(methyl(4-(4-methylpiperazin-1-yl)phenyl)amino)pyridin-2-yl)oxy)phenyl)acrylamide

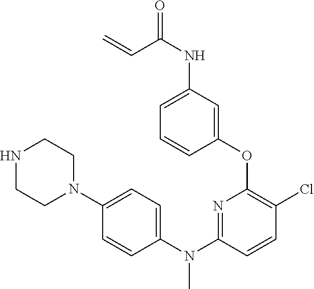 [SMILES: C=CC(=O)Nc1cccc(Oc2nc(N(C)c3ccc(N4CCNCC4)cc3)ccc2Cl)c1]

Prepared according to compound 7 using tert-butyl 4-(4-((5-chloro-6-(3-nitrophenoxy)pyridin-2-yl)(methyl)amino)phenyl)piperazine-1-carboxylate in step c to afford the product as a gray solid, ¹H NMR (400 MHz, DMSO-d₆) δ 10.25 (s, 1H), 7.60 (bs, 1H), 7.60 (t, J=2.0 Hz, 1H), 7.57 (d, J=8.8 Hz, 1H), 7.43-7.41 (m, 1H), 7.34 (t, =8.0 Hz, 1H), 7.16-7.13 (m, 2H), 7.02-6.98 (m, 2H), 6.89-6.86 (m, 1H), 6.43 (dd, J=16.8, 10.0 Hz, 1H), 6.26 (dd, 16.8, 2.0 Hz, 1H), 6.07 (d, J=8.8 Hz, 2H), 5.77 (dd, J=10.0, 2.0 Hz, 1H), 3.32 (bs, 4H), 3.24 (bs, 4H), 3.06 (s, 3H). LC-MS C₂₆H₂₉ClN₅O₂ (M+H)⁺ calculated mass, 464.1853; found, 464.47.

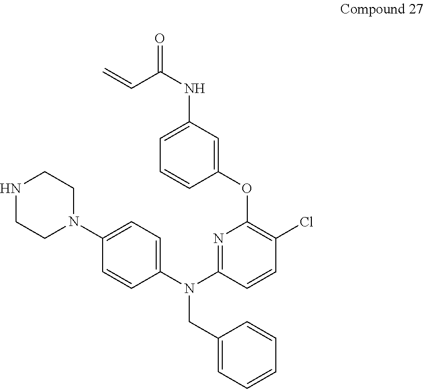 [SMILES: C=CC(=O)Nc1cccc(Oc2nc(N(Cc3ccccc3)c3ccc(N4CCNCC4)cc3)ccc2Cl)c1]

### N-(3-((6-(benzyl(4-(piperazin-1-yl)phenyl)amino)-3-chloropyridin-2-yl)oxy)phenyl)acrylamide

Prepared according to compound 26 using (bromomethyl)benzene instead of iodomethane to afford the product (16 mg) as a gray solid. ¹H NMR (400 MHz, DMSO-d₆) δ 10.24 (s, 1H), 8.70 (bs, 1H), 7.65 (t, J=2.0 Hz, 1H), 7.55 (d, J=8.8 Hz, 1H), 7.38-7.35 (m, 1H), 7.30 (t, J=8.4 Hz, 1H), 7.12-7.08 (m, 2H), 7.06-7.03 (m, 2H), 7.00-6.97 (m, 2H), 6.87-6.83 (m, 3H), 6.44 (dd, J=16.8, 10.0 Hz, 1H), 6.28 (dd, J=16.8, 2.0 Hz, 1H), 5.94 (d, J=8.4 Hz, 1H), 5.79 (dd, J=10.0, 2.0 Hz, 1H), 4.71 (s, 2H), 3.78 (s, 3H), 3.32 (bs, 4H), 3.22 (bs, 4H). LC-MS C₃₁H₃₁ClN₅O₂ (M+H)⁺ calculated mass, 540.2166; found, 540.61.

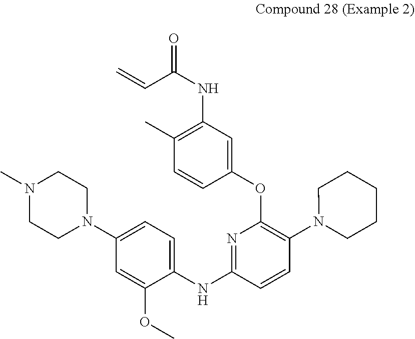 [SMILES: C=CC(=O)Nc1cc(Oc2nc(Nc3ccc(N4CCN(C)CC4)cc3OC)ccc2N2CCCCC2)ccc1C]

### N-(5-((6-((2-methoxy-4-(4-methylpiperazin-1-yl)phenyl)amino)-3-(piperidin-1-yl)pyridin-2-yl)oxy)-2-methylphenyl)acrylamide

¹H NMR (400 MHz, DMSO-d₆) δ 9.87 (s, 1H), 9.54 (s, 1H), 7.73 (s, 1H), 7.51 (s, 1H), 7.32-7.28 (m, 2H), 6.95 (d, J=8.4 Hz, 1H), 6.62-6.55 (m, 3H), 6.23 (dd, J=17.2, 2.0 Hz, 1H), 6.09 (dd, J=8.8, 2.4 Hz, 1H), 5.75 (dd, J=10.0, 2.0 Hz, 1H), 3.78 (s, 3H), 3.72 (d, J==12.8 Hz, 2H), 3.52 (d, J=12.0 Hz, 2H), 3.42 (bs, 2H), 3.18-3.10 (m, 2H), 2.87 (s, 3H), 2.91-2.84 (m, 2H), 2.28 (s, 3H), 1.86 (bs, 4H), 1.59 (bs, 2H). HRMS C₃₂H₄₀N₆O₃ (M+H)⁺ calculated mass, 557.3240; found, 557.3245.

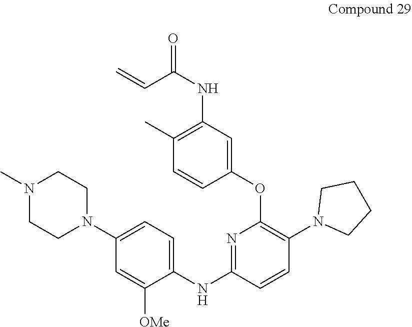 [SMILES: C=CC(=O)Nc1cc(Oc2nc(Nc3ccc(N4CCN(C)CC4)cc3OC)ccc2N2CCCC2)ccc1C]

### N-(5-((6-((2-methoxy-4-(4-methylpiperazin-1-yl)phenyl)amino)-3-(pyrrolidin-1-yl)pyridin-2-yl)oxy)-2-methylphenyl)acrylamide

Prepared according to example 2 (scheme 2) using pyrrolidine in step b to afford the title compound (25 mg) as a gray solid. NMR (400 MHz, DMSO-d₆) δ 9.45 (s, 1H), 7.36 (d, J=8.8 Hz, 1H), 7.14 (d, J=8.8 Hz, 1H), 7.31 (s, 1H), 7.22 (d, J=8.0 Hz, 1H), 7.12 (d, J=8.8 Hz, 1H), 6.80 (dd, J=8.0, 2.4 Hz, 1H), 6.54 (d, J=8.4 Hz, 1H), 7.51 (d, J=2.4 Hz, 1H), 6.21 (dd, J=17.2, 2.0 Hz, 1H), 6.05 (dd, J=8.8, 2.4 Hz, 1H), 5.73 (dd, J=10.0, 2.0 Hz, 1H), 3.76 (s, 3H), 3.17 (t, J=6.0 Hz, 1H), 3.00 (t, =5.2 Hz, 1H), 2.43 (t, J=5.2 Hz, 1H), 2.25 (s, 3H), 2.21 (s, 3H), 1.86-1.83 (m, 4H). HRMS C₃₁H₃₉N₆O (M+H)⁺ calculated mass, 543.3084; found, 543.3088.

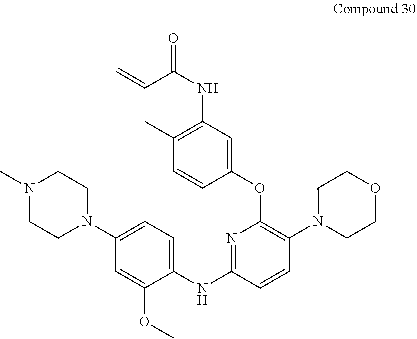 [SMILES: C=CC(=O)Nc1cc(Oc2nc(Nc3ccc(N4CCN(C)CC4)cc3OC)ccc2N2CCOCC2)ccc1C]

### N-(5-((6-((2-methoxy-4-(4-methylpiperazin-1-yl)phenyl)amino)-3-morpholinopyridin-2-yl)oxy)-2-methylphenyl)acrylamide

Prepared according to example 2 (scheme 2) using morpholine in step b to afford the title compound (23 mg) as a gray solid. ¹H NMR (400 MHz, DMSO-d₆) δ 9.46 (s, 1H), 7.53 (s, 1H), 7.39 (s, 1H), 7.32-7.23 (m, 3H), 6.83 (dd, J=8.4, 2.8 Hz, 1H), 6.58 (dd, J=17.6, 10.8 Hz, 1H), 6.53-6.51 (m, 2H), 6.21 (dd, J=17.2, 2.0 Hz, 1H), 6.02 (dd, J=8.8, 2.4 Hz, 1H), 5.73 (dd, J=10.4, 2.0 Hz, 1H), 3.75 (s, 3H), 3.70 (t, J=4.8 Hz, 4H), 3.02 (bs, 4H), 2.95 (t, J=4.8 Hz, 4H), 2.46 (bs, 4H), 2.27 (s, 3H), 2.24 (s, 3H). HRMS C₃₁H₃₈N₆O₄ (M+H)⁺ calculated mass, 559.3033; found, 559.3063.

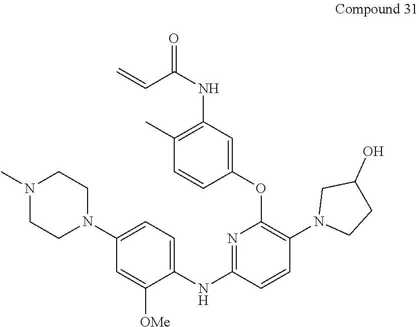 [SMILES: C=CC(=O)Nc1cc(Oc2nc(Nc3ccc(N4CCN(C)CC4)cc3OC)ccc2N2CCC(O)C2)ccc1C]

### N-(5-((3-(3-hydroxypyrrolidin-1-yl)-6-((2-methoxy-4-(4-methylpiperazin-1-yl)phenyl)amino)pyridin-2-yl)oxy)-2-methylphenyl)acrylamide

### (±)-3-(tert-Butyldimethylsilyloxy)pyrrolidine hydrochloride

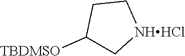

(±)-3-(tert-Butyldimethylsilyloxy)pyrrolidine hydrochloride was prepared following the reference: Tetrahedron 68 (2012) 7295-7301.

### N-(5-((3-(3-((tert-butyldimethylsilyl)oxy)pyrrolidin-1-yl)-6-((2-methoxy-4-(4-methylpiperazin-1-yl)phenyl)amino)pyridin-2-yl)oxy)-2-methylphenyl)acrylamide

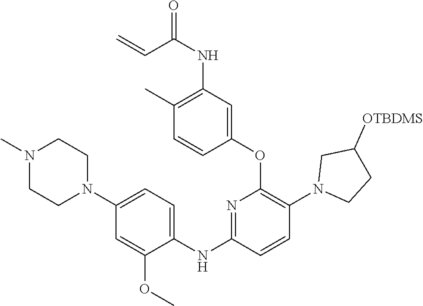 [SMILES: C=CC(=O)Nc1cc(Oc2nc(Nc3ccc(N4CCN(C)CC4)cc3OC)ccc2N2CCC(O[Si](C)(C)C(C)(C)C)C2)ccc1C]

Prepared according to example 2 (scheme 2) using (±)-3-(tert-Butyldimethylsilyloxy)pyrrolidine hydrochloride in step b to afford N-(5-((3-(3-((tert-butyldimethylsilyl)oxy)pyrrolidin-1-yl)-6-((2-methoxy-4-(4-methylpiperazin-1-yl)phenyl)amine)pyridin-2-yl)oxy)-2-methylphenyl)acrylamide. LC-MS m/z: 673.78 (M+1).

### N-(5-((3-(3-hydroxypyrrolidin-1-yl)-6-((2-methoxy-4-(4-methylpiperazin-1-yl)phenyl)amino)pyridin-2-yl)oxy)-2-methylphenyl)acrylamide

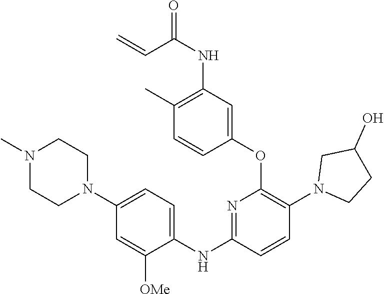 [SMILES: C=CC(=O)Nc1cc(Oc2nc(Nc3ccc(N4CCN(C)CC4)cc3OC)ccc2N2CCC(O)C2)ccc1C]

To a solution of N-(5-((3-(3-((tert-butyldimethylsilyl)oxy)pyrrolidin-1-yl)-methoxy-4-(4-methylpiperazin-1-yl)phenyl)amino)pyridin-2-yl)oxy)-2-methylphenyl)acrylamide (1 eq) in tetrahydrofuran was added tetrabutylammonium fluoride (1M tetrahydrofuran solution, 5 eq) on an ice bath, which was stirred for 4 hours at room temperature. Water was added to the reaction solution, which was then extracted with ethyl acetate three times. The extract was dried over anhydrous sodium sulfate, and the solvent was evaporated under a reduced pressure. The obtained residue was dissolved in dichloromethane (2 ml) and TEA (0.25 ml) to afford the TEA salt of title compound. Dichloromethane and excess TEA was removed in vacuo and resolved in methanol and purified by reverse-phase HPLC eluting with acetonitrile containing 0.15%) TEA to give title compound (9 mg) as a gray solid. ¹H NMR (400 MHz, DMSO-d₆) δ 9.73 (s, 1H), 9.52 (s, 1H), 7.44-7.14 (m, 4H), 6.98-6.74 (m, 2H), 6.57 (dd, J=16.8, 10.0 Hz, 1H), 6.22 (dd, =16.8, 2.0 Hz, 1H), 5.74 (dd, J=10.0, 2.0 Hz, 1H), 3.76 (s, 3H), 3.54-3.49 (m, 4H), 3.11 (bs, 4H), 2.86 (d, J=3.6 Hz, 3H), 2.25 (s, 3H). HRMS C₃₁H₃₈N₆O₄ (M+H)⁺ calculated mass, 559.3033; found, 559.3017.

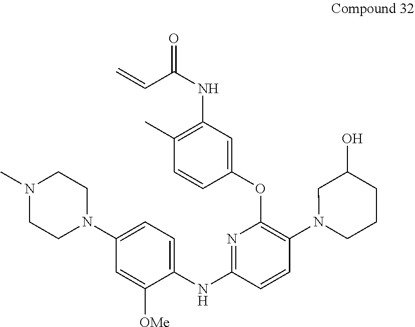 [SMILES: C=CC(=O)Nc1cc(Oc2nc(Nc3ccc(N4CCN(C)CC4)cc3OC)ccc2N2CCCC(O)C2)ccc1C]

### N-(5-((3-(3-hydroxypiperidin-1-yl)-6-((2-methoxy-4-(4-methylpiperazin-1-yl)phenyl)amino)pyridin-2-yl)oxy)-2-methylphenyl)acrylamide

### (±)3-(tert-butyldimethylsilyl)oxy)piperidine

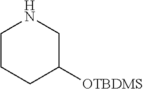 [SMILES: CC(C)(C)[Si](C)(C)OC1CCCNC1]

(±)3-((tert-butyldimethylsilyl)oxy)piperidine was prepared according to methods mentioned in preparation of (±)-3-(tert-Butyldimethylsilyloxy)pyrrolidine hydrochloride in compound 34. LC-MS m/z: 216.28 (M+1).

### N-(5-((3-(3-hydroxypiperidin-1-yl)-6-((2-methoxy-4-(4-methylpiperazin-1-yl)phenyl)amino)pyridin-2-yl)oxy)-2-methylphenyl)acrylamide

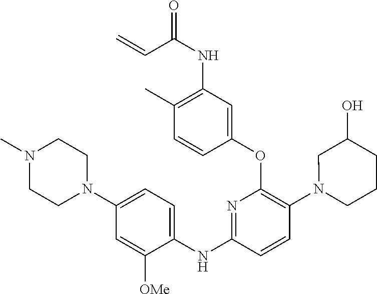 [SMILES: C=CC(=O)Nc1cc(Oc2nc(Nc3ccc(N4CCN(C)CC4)cc3OC)ccc2N2CCCC(O)C2)ccc1C]

Prepared according to compound 31 (scheme 2) using (±)3-((tert-butyldimethylsilyl)oxy)piperidine in step b to afford the title compound (12 mg) as a gray solid. ¹H NMR (400 MHz, DMSO-d₆) δ 965 (s, 1H), 951 (s, 1H), 7.40-7.36 (m, 3H), 7.26 (d, J=8.0 Hz, 1H), 6.88 (d, J=8.0 Hz, 1H), 6.60-6.53 (m, 3H), 6.23 (dd, J=16.8, 2.0 Hz, 1H), 6.09 (dd, J=8.8, 2.4 Hz, 1H), 5.75 (d, J=10.0, 1H), 3.78 (s, 3H), 3.71-3.68 (m, 2H), 3.53-3.50 (m, 2H), 3.40-3.35 (m, 2H), 3.18-3.10 (m, 3H), 2.88-2.83 (m, 5H), 2.27 (s, 3H), 1.89-1.81 (m, 2H), 1.66-1.58 (m, 2H), 1.29-1.23 (m, 2H). HRMS C₃₂H₄₀N₆O₄ (M+H)⁺ calculated mass, 573.3189; found, 573.3195.

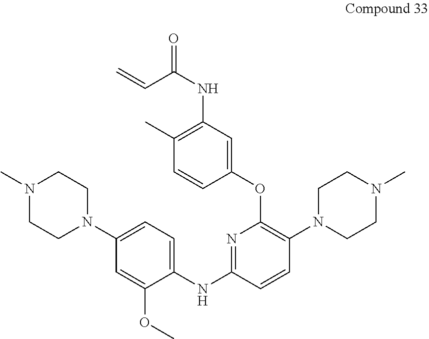

### N-(5-((6-((2-methoxy-4-(4-methylpiperazin-1-yl)phenyl)amino)-3-(4-methylpiperazin-1-yl)pyridin-2-yl)oxy)-2-methylphenyl)acrylamide

Prepared according to example 2 (scheme 2) using 1-methylpiperazine in step b to afford the title compound (18 mg) as a gray solid. ¹H NMR (400 MHz, MeOH) & 7.51 (5, 1H), 7.39 (s, 1H), 7.31-7.28 (m, 2H), 6.91 (dd, =8.0, 2.4 Hz, 1H), 6.64-6.58 (m, 1H), 6.52 (dd, J=16.8, 10.4 Hz, 1H), 6.53-6.46 (m, 1H), 6.35 (dd, =16.8, 2.0 Hz, 1H), 6.25-6.17 (m, 1H), 5.79 (dd, J=10.4, 2.0 Hz, 1H), 3.84 (s, 3H), 3.56-3.52 (m, 4H), 3.49-3.46 (m, 4H), 3.24-3.19 (m, 4H), 2.97 (s, 3H), 2.94 (s, 3H), 2.33 (s, 3H). HRMS C₃₂H₄₂N₇O₃ (M+H)⁺ calculated mass, 572.3349; found, 572.3361.

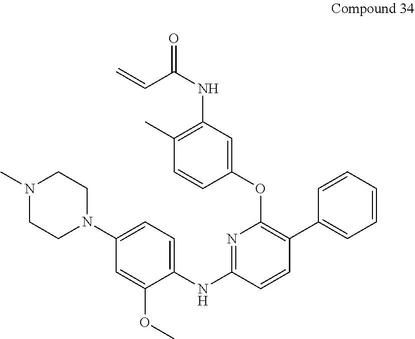 [SMILES: C=CC(=O)Nc1cc(Oc2nc(Nc3ccc(N4CCN(C)CC4)cc3OC)ccc2-c2ccccc2)ccc1C]

### N-(5-((6-((2-methoxy-4-(4-methylpiperazin-1-yl)phenyl)amino)-3-phenylpyridin-2-yl)oxy)-2-methylphenyl)acrylamide

### 6-chloro-2-(4-methyl-3-nitrophenoxy)-3-phenylpyridine

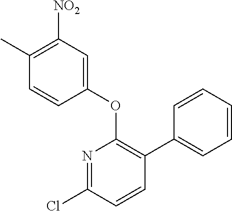 [SMILES: Cc1ccc(Oc2nc(Cl)ccc2-c2ccccc2)cc1[N+](=O)[O-]]

Tetrakistriphenylphosphine palladium (116 mg, 0.1 mmol), phenylboronic acid (146 mg, 1.2 mmol) and a 2 M sodium carbonate solution (1 ml) were sequentially added to a solution of intermediate 2a (344 mg, 1 mmol) in 1,2-dimethoxyethane (5 nil) in a nitrogen atmosphere, and the mixture was stirred at 80° C. for 8 hours. Water was added to the reaction solution at room temperature, followed by extraction with ethyl acetate three times. The combined organic phase was washed with brine, dried over anhydrous sodium sulfate and filtered. The solvent was then evaporated under reduced pressure. The resulting residue was purified by silica gel column chromatography to give title product (304 mg, yield: 89%). LC-MS m/z: 341.23 (M+1).

### N-(5-((6-((2-methoxy-4-(4-methylpiperazin-1-yl)phenyl)amino)-3-phenylpyridin-2-yl)oxy)-2-methylphenyl)acrylamide

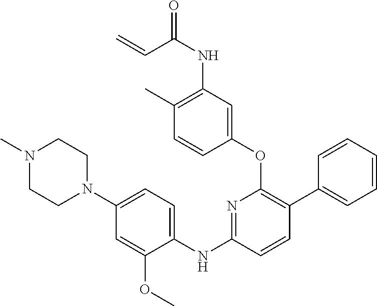 [SMILES: C=CC(=O)Nc1cc(Oc2nc(Nc3ccc(N4CCN(C)CC4)cc3OC)ccc2-c2ccccc2)ccc1C]

Prepared according to example 2 using 6-chloro-2-(4-methyl-3-nitrophenoxy)-3-phenylpyridine in step c to afford the title compound (22 mg) as a gray solid. ¹H NMR (400 MHz, DMSO-d₆) δ 9.47 (s, 1H), 8.10 (s, 1H), 7.67-7.60 (m, 3H), 7.45-7.38 (m, 4H), 7.30-7.24 (m, 2H), 6.88 (dd, J=8.4, 2.4 Hz, 1H), 6.70 (d, J=8.4 Hz, 1H), 6.62 (d, J=2.4 Hz, 1H), 6.57 (dd, J=16.8, 10.0 Hz, 1H), 6.21 (dd, J=16.8, 2.0 Hz, 1H), 6.04 (dd, J=8.8, 2.4 Hz, 1H), 5.74 (dd, J=10.0, 2.0 Hz, 1H), 3.80 (s, 3H), 3.72 (d, J=12.8 Hz, 2H), 3.52 (d, J=12.0 Hz, 2H), 3.19-3.11 (m, 2H), 2.92-2.86 (m, 5H), 2.26 (s, 3H). HRMS C₃₃H₃₅N₅O₃ (M+H)⁺ calculated mass, 550.2818; found, 550.2834.

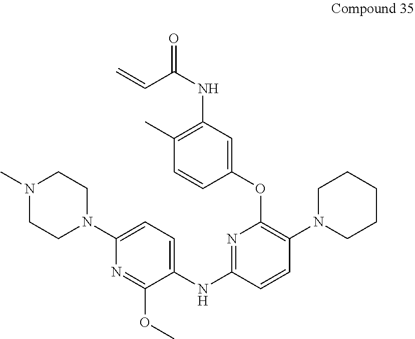 [SMILES: C=CC(=O)Nc1cc(Oc2nc(Nc3ccc(N4CCN(C)CC4)nc3OC)ccc2N2CCCCC2)ccc1C]

### N-(5-((6-((2-methoxy-6-(4-methylpiperazin-1-yl)pyridin-3-yl)amino)-3-(piperidin-1-yl)pyridin-2-yl)oxy)-2-methylphenyl)acrylamide

### 2-methoxy-6-(4-methylpiperazin-1-yl)pyridin-3-amine

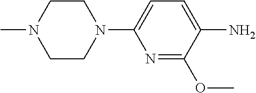 [SMILES: COc1nc(N2CCN(C)CC2)ccc1N]

2-methoxy-6-(4-methylpiperazin-1-yl)pyridin-3-amine was prepared following the patent: CN105218561, 2016.

### N-(5-((6-((2-methoxy-6-(4-methylpiperazin-1-yl)pyridin-3-yl)amino)-3-(piperidin-1-yl)pyridin-2-yl)oxy)-2-methylphenyl)acrylamide

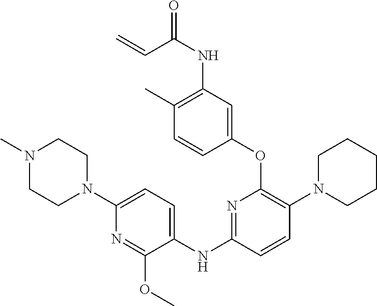 [SMILES: C=CC(=O)Nc1cc(Oc2nc(Nc3ccc(N4CCN(C)CC4)nc3OC)ccc2N2CCCCC2)ccc1C]

Prepared according to example 2 (scheme) using 2-methoxy-6-(4-methylpiperazin-1-yl)pyridin-3-amine in step c to afford the title compound (19 mg) as a gray solid, ¹H NMR (400 MHz, MeOH) δ 7.81 (d, J=8.8 Hz, 1H), 7.66 (d, J=8.4 Hz, 1H), 7.45 (d, J=2.4 Hz, 1H), 7.35 (d, J=8.4 Hz, 1H), 7.02 (dd, J=8.0, 2.4 Hz), 6.57 (d, J=8.0 Hz, 1H), 6.54 (dd, J=16.8, 10.0 Hz, 1H), 6.36 (dd, J=16.8, 1.6 Hz, 1H), 6.00 (d, J=8.8 Hz, 1H), 5.80 (dd, 10.0, 1.6 Hz, 1H), 4.33 (bs, 2H), 3.90 (s, 3H), 3.72 (bs, 4H), 3.58 (bs, 2H), 3.13 (bs, 4H), 2.95 (s, 3H), 2.36 (s, 3H), 2.06 (bs, 4H), 1.79 (bs, 2H). LC-MS C₃₁H₄₀N₇O₃ (M+H)⁺ calculated mass, 558.3193 found, 558:32.

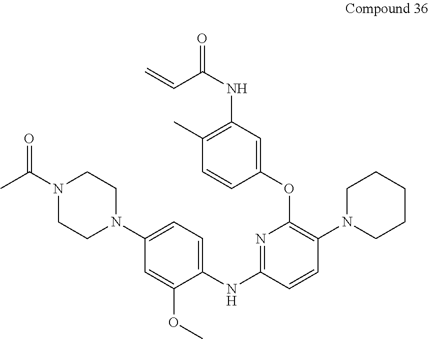 [SMILES: C=CC(=O)Nc1cc(Oc2nc(Nc3ccc(N4CCN(C(C)=O)CC4)cc3OC)ccc2N2CCCCC2)ccc1C]

### N-(5-((6-((4-(4-acetylpiperazin-1-yl)-2-methoxyphenyl)amino)-3-(piperidin-1-yl)pyridin-2-yl)oxy)-2-methylphenyl)acrylamide

Prepared according to example 2 using 1-(4-(4-amino-3-methoxyphenyl)piperazin-1-yl)ethan-1-one in step c to afford the title compound (25 mg) as a gray solid. ¹H NMR (400 MHz, DMSO-d₆) δ 9.51 (s, 1H), 7.58 (s, 1H), 7.30 (d, J=8.4 Hz, 1H), 7.26-7.23 (m, 1H), 6.96 (s, 1H), 6.64-6.57 (m, 2H), 6.61 (dd, J=16.8, 10.0 Hz, 1H), 6.23 (dd, J=17.2, 2.0 Hz, 1H), 6.06 (d, J=8.0 Hz, 1H), 5.76 (dd, =10.0, 2.0 Hz, 1H), 3.77 (s, 3H), 3.57-3.55 (m, 4H), 3.06-3.01 (m, 4H), 2.30 (s, 3H), 2.05 (s, 3H), 1.88 (bs, 4H), 1.53 (bs, 2H). LC-MS C₃₃H₄₁N₆O₄ (M+H)⁺ calculated mass, 585.3189; found, 585.44.

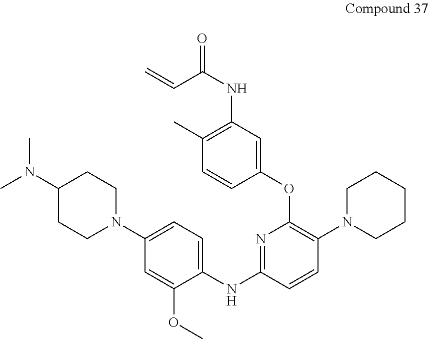 [SMILES: C=CC(=O)Nc1cc(Oc2nc(Nc3ccc(N4CCC(N(C)C)CC4)cc3OC)ccc2N2CCCCC2)ccc1C]

### N-(5-((6-((4-(4-(dimethylamino)piperidin-1-yl)-2-methoxyphenyl)amino)-3-(piperidin-1-yl)pyridin-2-yl)oxy)-2-methylphenyl)acrylamide

### 1-(4-amino-3-methoxyphenyl)-N,N-dimethylpiperidin-4-amine

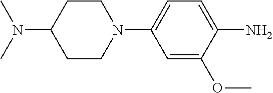 [SMILES: COc1cc(N2CCC(N(C)C)CC2)ccc1N]

1-(4-amino-3-methoxyphenyl)-N,N-dimethylpiperidin-4-amine was prepared following the patent: CN106905245, 2017.

### N-(5-((6-((4-(4-(dimethylamino)piperidin-1-yl)-2-methoxyphenyl)amino)-3-(piperidin-1-yl)pyridin-2-yl)oxy)-2-methylphenyl)acrylamide

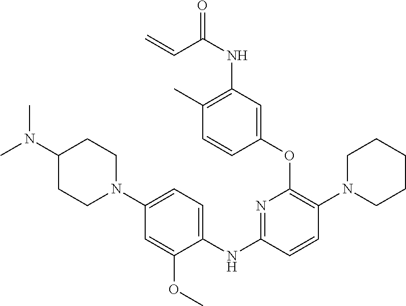 [SMILES: C=CC(=O)Nc1cc(Oc2nc(Nc3ccc(N4CCC(N(C)C)CC4)cc3OC)ccc2N2CCCCC2)ccc1C]

Prepared according to example 2 using 1-(4-amino-3-methoxyphenyl)-N,N-dimethylpiperidin-4-amine in step c to afford the title compound (22 mg) as a gray solid. ¹H NMR (400 MHz, DMSO-d₆) δ 9.91 (s, 1H), 9.56 (s, 1H), 7.52 (s, 1H), 7.30 (d, J=8.4 Hz, 1H), 7.26-7.24 (m, 1H), 6.95 (d, J=8.4 Hz, 1H), 6.61 (dd, J=17.2, 10.0 Hz, 1H), 6.61-6.57 (m, 2H), 6.23 (dd, J=17.2, 2.0 Hz, 1H), 6.10 (d, J=8.4 Hz, 1H), 5.76 (dd, J=10.0, 2.0 Hz, 1H), 3.77 (s, 3H), 3.72-3.69 (m, 3.28 (bs, 2H), 2.78 (s, 3H), 2.77 (s, 3H), 2.66 (bs, 2H), 2.30 (s, 3H), 2.09-2.06 (m, 2H), 1.86-1.73 (m, 7H), 1.55 (bs, 2H). LC-MS C₃₄H₄₅N₆O (M+H)⁺ calculated mass, 585.3553; found, 585.17.

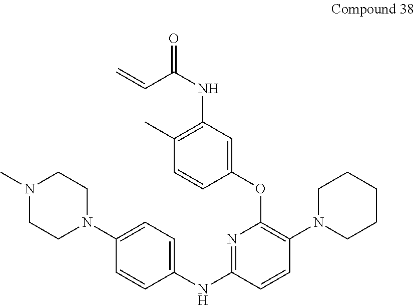 [SMILES: C=CC(=O)Nc1cc(Oc2nc(Nc3ccc(N4CCN(C)CC4)cc3)ccc2N2CCCCC2)ccc1C]

### N-(2-methyl-5-((6-((4-(4-methylpiperazin-1-yl)phenyl)amino)-3-(piperidin-1-yl)pyridin-2-yl)oxy)phenyl)acrylamide

Prepared according to example 2 using 4-(4-methylpiperazin-1-yl)aniline in step c to afford the title compound (13 mg) as a gray solid. ¹H NMR (400 MHz, DMSO-d₆) δ 9.85 (s, 1H), 9.58 (s, 1H), 7.49 (s, 1H), 7.31 (d, J=8.4 Hz, 1H), 7.12 (d, J=8.4 Hz, 2H), 6.95 (d, J=8.4 Hz, 1H), 6.64 (d, J=8.8 Hz, 2H), 6.58 (dd, J=16.8, 10.0 Hz, 1H), 6.46 (d, J=8.4 Hz, 1H), 6.22 (dd, J=16.8, 2.0 Hz, 1H), 5.75 (dd, J=10.0, 2.0 Hz, 1H), 3.61 (d, J=12.8 Hz, 2H), 3.51 (d, J=12.0 Hz, 2H), 3.18-3.10 (m, 2H), 2.86-2.80 (m, 5H), 2.30 (s, 3H), 1.81 (bs, 4H), 1.57 (bs, 2H). HRMS C₃₁H₃₉N₆O₂ (M+H)⁺ calculated mass, 527.3134; found, 527.3146.

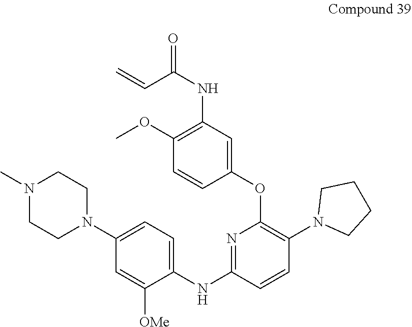 [SMILES: C=CC(=O)Nc1cc(Oc2nc(Nc3ccc(N4CCN(C)CC4)cc3OC)ccc2N2CCCC2)ccc1OC]

### N-(2-methoxy-5-((6-((2-methoxy-4-(4-methylpiperazin-1-yl)phenyl)amino)-3-(pyrrolidin-1-yl)pyridin-2-yl)oxy)phenyl)acrylamide

Prepared according to example 2 using 4-methoxy-3-nitrophenol in step a and pyrrolidine in step b to afford the title compound (8 mg) as a gray solid. ¹H NMR (400 MHz, DMSO-d₆) δ 9.48 (s, 1H), 8.07-8.03 (m, 1H), 7.45-7.40 (m, 1H), 7.12-7.08 (m, 1H); 6.90-6.86 (m, 1H), 6.75 (dd, J=16.8, 10.0 Hz, 1H), 6.61-6.55 (m, 2H), 6.20 (dd, J=17.2, 2.0 Hz, 1H), 6.08-6.04 (m, 1H), 5.72 (dd, J=10.0, 2.0 Hz, 1H), 3.89 (s, 3H), 3.78 (s, 3H), 3.52-3.49 (m, 4H), 3.13-3.08 (m, 4H), 2.87-2.80 (m, 5H), 1.96 (bs, 4H). HRMS C₃₁H₃₉N₆O₄ (M+H)⁺ calculated mass, 559.3033 found, 559.3047.

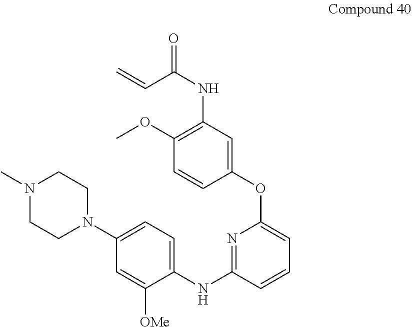 [SMILES: C=CC(=O)Nc1cc(Oc2cccc(Nc3ccc(N4CCN(C)CC4)cc3OC)n2)ccc1OC]

### N-(2-methoxy-5-((6-((2-methoxy-4-(4-methylpiperazin-1-yl)phenyl)amino)pyridin-2-yl)oxy)phenyl)acrylamide

In purification of compound 39 by reverse-phase HPLC, we obtained compound 40 at the same time. ¹H NMR (400 MHz, DMSO-d₆) δ 9.47 (s, 1H), 8.01 (d, J=2.8 Hz, 1H), 7.92 (s, 1H), 7.57 (d, J=8.8 Hz, 1H), 7.47 (t, J=8.0 Hz, 1H), 7.09 (d, J=8.8 Hz, 1H), 6.85 (dd, J=8.8, 2.8 Hz, 1H), 6.75 (dd, J=16.8, 10.0 Hz, 1H), 6.63 (d, J=2.8 Hz, 1H), 6.50 (d, J=8.0 Hz, 1H), 6.22 (dd, J=8.8, 2.4 Hz, 1H), 6.20 (dd, J=16.8, 2.0 Hz, 1H), 6.12 (d, J=7.6 Hz, 1H), 5.71 (dd, J=10.0, 2.0 Hz, 1H), 3.89 (s, 3H), 3.79 (s, 3H), 3.73 (d, J=13.2 Hz, 2H), 3.51 (d, J=12.8 Hz, 2H), 3.19-3.11 (m, 2H), 2.91-2.84 (m, 5H). HRMS C₂₇H₃₂N₅O₄ (M+H)⁺ calculated mass, 490.2454; found, 490.2463.

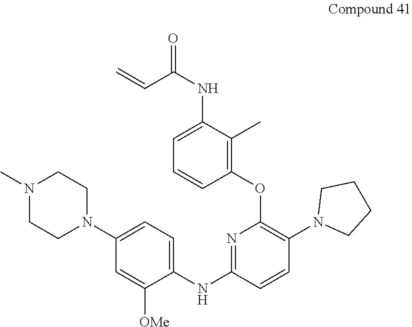 [SMILES: C=CC(=O)Nc1cccc(Oc2nc(Nc3ccc(N4CCN(C)CC4)cc3OC)ccc2N2CCCC2)c1C]

### N-(3-((6-((2-methoxy-4-(4-methylpiperazin-1-yl)phenyl)amino)-3-(pyrrolidin-1-yl)pyridin-2-yl)oxy)-2-methylphenyl)acrylamide

Prepared according to example 2 using 2-methyl-3-nitrophenol in step a and pyrrolidine in step b to afford the title compound (12 mg) as a gray solid, ¹H NMR (400 MHz, DMSO-d₆) δ 9.57 (s, 1H), 7.59 (d, J=8.8 Hz, 1H), 7.28-7.21 (m, 2H), 6.96-6.90 (m, 1H), 6.67-6.54 (m, 2H), 6.61 (dd, J=17.2, 10.0 Hz, 1H), 6.28 (dd, J=17.8, 2.0 Hz, 1H), 6.12-6.06 (m, 1H), 5.78 (dd, J=10.0, 2.0 Hz, 1H), 3.78 (s, 3H), 3.52-3.49 (m, 4H), 3.16-3.07 (m, 2H), 2.87-2.80 (m, 5H), 2.06 (s, 3H), 1.98 (bs, 4H). HRMS C₃₁H₃₉N₆O₃ (M+H)⁺ calculated mass, 543.3084; found, 543.3085.

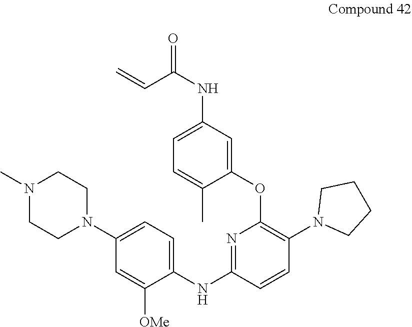 [SMILES: C=CC(=O)Nc1ccc(C)c(Oc2nc(Nc3ccc(N4CCN(C)CC4)cc3OC)ccc2N2CCCC2)c1]

### N-(3-((6-((2-methoxy-4-(4-methylpiperazin-1-yl)phenyl)amino)-3-(pyrrolidin-1-yl)pyridin-2-yl)oxy)-4-methylphenyl)acrylamide

Prepared according to example 2 using 2-methyl-5-nitrophenol in step a and pyrrolidine in step b to afford the title compound (13 mg) as a gray solid. ¹H NMR (400 MHz, DMSO-d₆) δ 10.19 (s, 1H), 9.64 (s, 1H), 7.58-7.55 (m, 1H), 7.39-7.22 (m, 3H), 6.67-6.54 (m, 2H), 6.39 (dd, J=16.8, 10.0 Hz, 1H), 6.23 (dd, J=16.8, 2.0 Hz, 1H), 6.04-5.94 (m, 1H), 5.74 (dd, J=10.0, 2.0 Hz, 1H), 3.77 (s, 3H), 3.51-3.48 (m, 4H), 3.15-3.07 (m, 2H), 2.86 (d, J=4.4 Hz, 3H), 2.82-2.79 (m, 2H), 2.09 (s, 3H), 1.95 (bs, 4H). HRMS C₃₁H₃₈N₆O₃ (M+H)⁺ calculated mass, 543.3084; found, 543.3091.

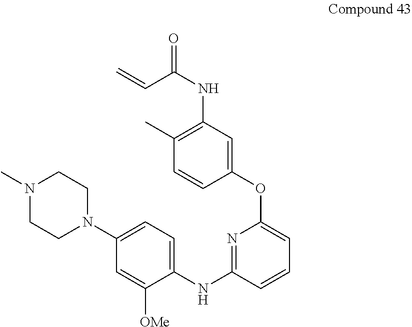 [SMILES: C=CC(=O)Nc1cc(Oc2cccc(Nc3ccc(N4CCN(C)CC4)cc3OC)n2)ccc1C]

### N-(5-((6-((2-methoxy-4-(4-methylpiperazin-1-yl)phenyl)amino)pyridin-2-yl)oxy)-2-methylphenyl)acrylamide

In purification of compound 29 by reverse-phase HPLC, we obtained compound 43 at the same time. ¹H NMR (400 MHz, MeOH) δ 7.64 (s, 1H), 7.49 (s, 1H), 7.29-7.28 (m, 2H), 6.93 (dd, J=8.0, 2.4 Hz, 1H), 6.68-6.63 (m, 1H), 6.51 (dd, J=16.8, 10.0 Hz, 1H), 6.35 (dd, J=16.8, 2.0 Hz, 1H), 6.24-6.15 (m, 1H), 5.79 (dd, J=10.0, 2.0 Hz, 1H), 3.91-3.81 (bs, 4H), 3.64-3.57 (bs, 4H), 2.97 (s, 3H), 2.30 (s, 3H). HRMS C₂₇H₃₂N₅O₃(M+H)⁺ calculated mass, 474.2505; found, 474.2515.

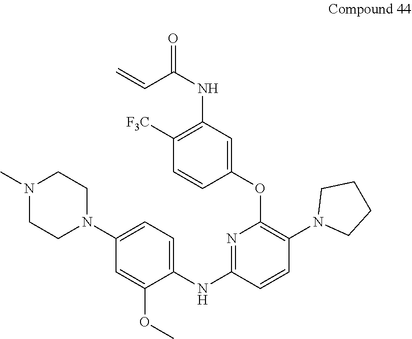 [SMILES: C=CC(=O)Nc1cc(Oc2nc(Nc3ccc(N4CCN(C)CC4)cc3OC)ccc2N2CCCC2)ccc1C(F)(F)F]

### N-(5-((6-((2-methoxy-4-(4-methylpiperazin-1-yl)phenyl)amino)-3-(pyrrolidin-1-yl)pyridin-2-yl)oxy)-2-(trifluoromethyl)phenyl)acrylamide

### 3-bromo-6-chloro-2-(3-nitro-4-(trifluoromethyl)phenoxy)pyridine

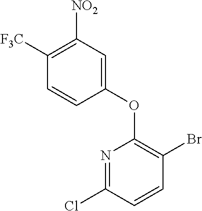 [SMILES: O=[N+]([O-])c1cc(Oc2nc(Cl)ccc2Br)ccc1C(F)(F)F]

Prepared according to the method of step a mentioned in scheme 2 using 3-nitro-4-(trifluoromethyl)phenol and 3-promo-2,6-dichloropyridine as starting material. LC-MS m/z: 396.90 (M+1).

### 5-((3-bromo-6-chloropyridin-2-yl)oxy)-2-(trifluoromethyl)aniline

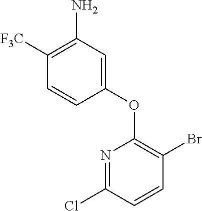 [SMILES: Nc1cc(Oc2nc(Cl)ccc2Br)ccc1C(F)(F)F]

Prepared according to the method of step c mentioned in scheme 1. LC-MS m/z: 367.08 (M+1).

### tert-butyl (5-((3-bromo-6-chloropyridin-2-yl)oxy)-2-(trifluoromethyl)phenyl)carbamate

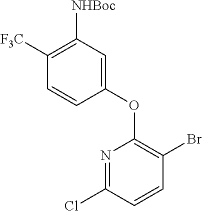 [SMILES: CC(C)(C)OC(=O)Nc1cc(Oc2nc(Cl)ccc2Br)ccc1C(F)(F)F]

5-((3-bromo-6-chloropyridin-2-yl)oxy)-2-(trifluoromethyl)aniline (284 mg, 0.77 mmol) was dissolved in THF (4 ml), (Boc)₂O (387 mg, 1.78 mmol), DMAP (9 mg, 0.077 mmol) and TEA (195 mg, 1.93 mmol) was added, the reaction mixture was stirred at room temperature overnight. The solvent was removed under reduced pressure and the residue was partitioned between dichloromethane and saturated aqueous sodium bicarbonate solution. The aqueous phase was extracted with DCM three times. The combined organic phase was dried over anhydrous sodium sulfate and concentrated in vacuo. The residue was purified by silica gel chromatography to afford the solid product (235 mg, yield: 65%). LC-MS m/z: 467.35).

### tert-butyl (3-((6-((2-methoxy-4-(4-methylpiperazin-1-yl)phenyl)amino)-3-(pyrrolidin-1-yl)pyridin-2-yl)oxy)phenyl)carbamate

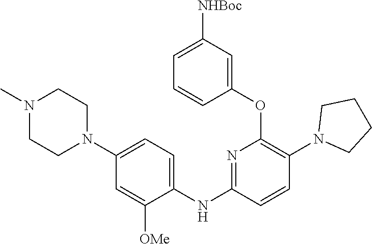 [SMILES: COc1cc(N2CCN(C)CC2)ccc1Nc1ccc(N2CCCC2)c(Oc2cccc(NC(=O)OC(C)(C)C)c2)n1]

Prepared according to the method of step b and c mentioned in scheme 2 using pyrrolidine in step b and tert-butyl (5-((3-bromo-6-chloropyridin-2-yl)oxy)-2-(trifluoromethyl)phenyl) carbamate in step c to afford the product. LC-MS m/z: 643.02 (M+1).

### 6-(3-aminophenoxy)-N-(2-methoxy-4-(4-methylpiperazin-1-yl)phenyl)-5-(pyrrolidin-1-yl)pyridin-2-amine

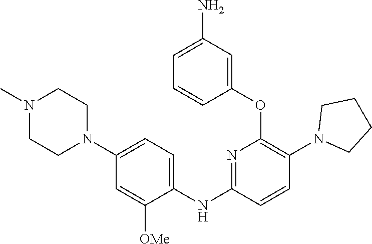 [SMILES: COc1cc(N2CCN(C)CC2)ccc1Nc1ccc(N2CCCC2)c(Oc2cccc(N)c2)n1]

tert-butyl(3-((6-((2-methoxy-4-(4-methylpiperazin-1-yl)phenyl)amino)-3-(pyrrolidin-1-yl)pyridin-2-yl)oxy)phenyl)carbamate was dissolved in DCM/TFA (2:1), the reaction mixture was stirred at room temperature for 2 hours. The solvent and excess TFA was removed in vacuo. The crude product was used for next transformation without further purification. LC-MS m/z: 542.58 (M+1).

### N-(5-((6-((2-methoxy-4-(4-methylpiperazin-1-yl)phenyl)amino)-3 (pyrrolidin-1-yl)pyridin-2-yl)oxy)-2-(trifluoromethyl)phenyl)acrylamide

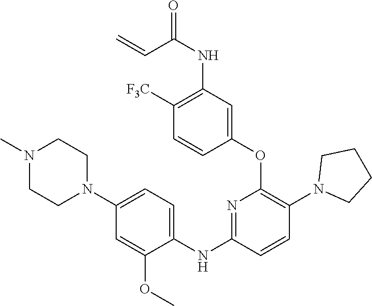 [SMILES: C=CC(=O)Nc1cc(Oc2nc(Nc3ccc(N4CCN(C)CC4)cc3OC)ccc2N2CCCC2)ccc1C(F)(F)F]

Prepared according to the method of step e mentioned in scheme 2 to afford the product (16 mg). ¹H NMR (400 MHz, DMSO-d₆) δ 9.76 (s, 1H), 7.75 (d, J=8.8 Hz, 1H), 7.47 (s, 1H), 7.32 (d, J=8.8 Hz, 1H), 7.26 (d, J=2.4 Hz, 1H), 7.19 (d, J=8.8 Hz, 1H), 7.10 (dd, J=8.8, 2.4 Hz, 1H), 6.62 (d, J=8.4 Hz, 1H), 6.53 (d, J=2.4 Hz, 1H), 6.54 (dd, J=17.2, 10.4 Hz, 1H), 6.23 (dd, J=17.2, 2.0 Hz, 1H), 6.10 (dd, 8.8, 2.8 Hz, 1H), 5.76 (dd, 10.4, 2.0 Hz, 1H), 3.76 (s, 3H), 3.16 (t, J=6.4 Hz, 4H), 3.00 (t, J=4.8 Hz, 4H), 2.42 (t, J=4.8 Hz, 4H), 2.21 (s, 3H), 1.85-1.82 (m, 4H). HRMS C₃₄H₃₅F₃N₆O₃ (M+H)⁺ calculated mass, 597.2801; found, 597.2819.

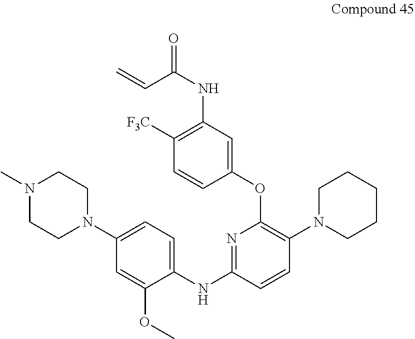 [SMILES: C=CC(=O)Nc1cc(Oc2nc(Nc3ccc(N4CCN(C)CC4)cc3OC)ccc2N2CCCCC2)ccc1C(F)(F)F]

### N-(5-((6-((2-methoxy-4-(4-methylpiperazin-1-yl)phenyl)amino)-3-piperidin-1-yl)pyridin-2-yl)oxy)-2-(trifluoromethyl)phenyl)acrylamide

Prepared according to compound 44 using piperidine instead of pyrrolidine to afford the title compound (30 mg) as a gray solid. ¹H NMR (400 MHz, DMSO-d₆) δ 9.82 (s, 1H), 9.64 (s, 1H), 7.79 (d, J=8.8 Hz, 1H), 7.38-7.30 (m, 2H), 7.22 (s, 1H), 6.65 (d, J=8.4 Hz, 1H), 6.62 (d, J=2.4 Hz, 1H), 6.54 (dd, J=17.2, 10.0 Hz, 1H), 6.23 (dd, J=17.2, 2.0 Hz, 1H), 6.12 (d, J=9.2 Hz, 1H), 5.78 (dd, J=10.4, 2.0 Hz, 1H), 3.78 (s, 3.68 (d, J=13.2 Hz, 2H), 3.52 (d, J=4.8 Hz, 2H), 3.16-3.08 (m, 2H), 2.89-2.82 (m, 5H), 1.67 (bs, 4H), 1.51 (bs, 2H), HRMS C₃₂H₃₈F₃N₆O₃ (M+H)⁺ calculated mass, 611.2957; found, 611.2964.

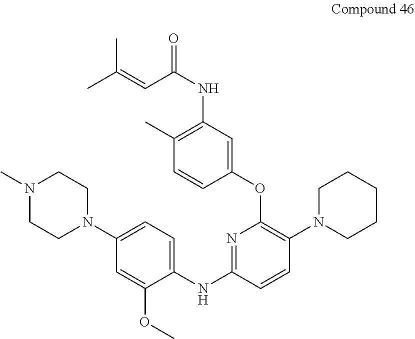 [SMILES: COc1cc(N2CCN(C)CC2)ccc1Nc1ccc(N2CCCCC2)c(Oc2ccc(C)c(NC(=O)C=C(C)C)c2)n1]

### N-(5-((6-((2-methoxy-4-(4-methylpiperazin-1-yl)phenyl)amino)-3-(piperidin-1-yl)pyridin-2-yl)oxy)-2-methylphenyl)-3-methylbut-2-enamide

Prepared according to example 2 using 3-methylbut-2-enoyl chloride in step e to afford the title compound (13 mg) as a gray solid. ¹H NMR (400 MHz, DMSO-d₆) δ 9.86 (s, 1H), 9.39 (s, 1H), 7.32-7.25 (m, 3H), 6.91-6.90 (m, 1H), 6.60-6.56 (m, 2H), 6.08 (d, J=8.8 Hz, 1H), 3.77 (s, 3H), 3.71 (d, J=13.2 Hz, 2H), 3.52 (d, J=12.0 Hz, 2H), 3.18-3.12 (m, 2H), 3.09 (s, 3H), 2.87 (s, 3H), 2.86 (s, 3H), 2.25 (s, 3H), 1.83 (bs, 4H), 1.85-1.76 (m, 7H), 1.55 (bs, 2H). LC-MS C₃₄H₄₅N₆O₃ (M+H)⁺ calculated mass, 585.3553, found, 585.54.

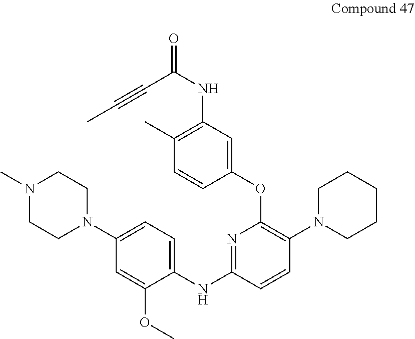 [SMILES: CC#CC(=O)Nc1cc(Oc2nc(Nc3ccc(N4CCN(C)CC4)cc3OC)ccc2N2CCCCC2)ccc1C]

### N-(5-((6-((2-methoxy-4-(4-methylpiperazin-1-yl)phenyl)amino)-3-(piperidin-1-yl)pyridin-2-yl)oxy)-2-methylphenyl)but-2-ynamide

A mixture of 6-(3-amino-4-methylphenoxy)-N-(2-methoxy-4-(4-methylpiperazin-1-yl)phenyl)-5-(piperidin-1-yl)pyridin-2-amine (Intermediate 2d, 60 mg, 0.12 mmol) and but-2-ynoic acid (11 mg, 0.13 mmol) in anhydrous acetonitrile (3 mL) was stirred at room temperature for 10 min. DMTMM (36 mg, 013 mmol) was added to the mixture and stirred overnight at room temperature. The solvent was removed, and water was added to the reaction solution at room temperature, followed by extraction with dichloromethane three times. The combined organic phase was washed with brine, dried over anhydrous sodium sulfate and filtered, then concentrated under reduced pressure. The obtained residue was dissolved in dichloromethane (2 ml) and TEA (0.25 ml) to afford the TEA salt of title compound. Dichloromethane and excess TFA was removed in vacuo and the residue dissolved in methanol and purified by reverse-phase HPLC eluting with acetonitrile containing 0.15‰ TEA to give title compound (22 mg) as a gray solid. ¹H NMR (400 MHz, DMSO-d₆) δ 10.09 (s, 1H), 9.81 (s, 1H), 7.28-7.20 (m, 3H), 6.94 (s, 1H), 6.60-6.56 (m, 2H), 6.09 (d, J=8.8 Hz, 1H), 3.77 (s, 3H), 3.72 (d, J=13.2 Hz, 2H), 3.54-3.51 (m, 2H), 3.18-3.10 (m, 2H), 2.90-2.84 (m, 5H), 2.24 (s, 3H), 2.04 (s, 3H), 1.77 (bs, 4H), 1.55 (bs, 2H). LC-MS C₃₃H₄₁N₆O₃ (M+H)⁺ calculated mass, 569.3240; found, 569.54.

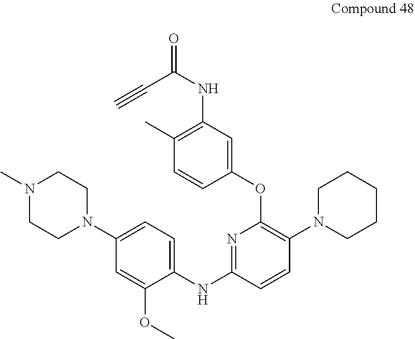 [SMILES: C#CC(=O)Nc1cc(Oc2nc(Nc3ccc(N4CCN(C)CC4)cc3OC)ccc2N2CCCCC2)ccc1C]

### N-(5-((6-((2-methoxy-4-(4-methylpiperazin-1-yl)phenyl)amino)-3-(piperidin-1-yl)pyridin-2-yl)oxy)-2-methylphenyl)propiolamide

### N-(5-((6-((2-methoxy-4-(4-methylpiperazin-1-yl)phenyl)amino)-3-(piperidin-1-yl)pyridin-2-yl)oxy)-2-methylphenyl)-3-(trimethylsilyl)propiolamide

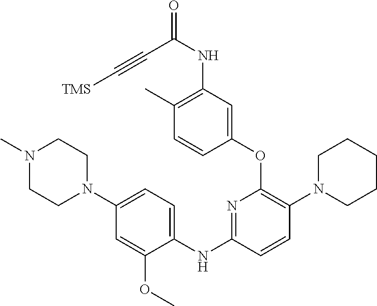 [SMILES: COc1cc(N2CCN(C)CC2)ccc1Nc1ccc(N2CCCCC2)c(Oc2ccc(C)c(NC(=O)C#C[Si](C)(C)C)c2)n1]

Prepared according to compound 47 using 3-(trimethylsilyl)propiolic acid in step e to obtain the title compound. LC-MS m/z: 627.53 (M+1).

### N-(5-((6-((2-methoxy-4-(4-methylpiperazin-1-yl)phenyl)amino)-3-(piperidin-1-yl)pyridin-2-yl)oxy)-2-methylphenyl)propiolamide

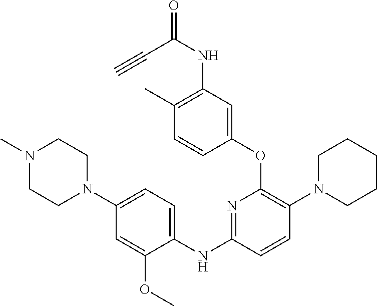 [SMILES: C#CC(=O)Nc1cc(Oc2nc(Nc3ccc(N4CCN(C)CC4)cc3OC)ccc2N2CCCCC2)ccc1C]

Prepared according to the methods mentioned in preparation of compound 13 to afford the title compound (12 mg) as a gray solid. ¹H NMR (400 MHz, DMSO-d₆) δ 10.35 (s, 1H), 9.73 (s, 1H), 7.32-7.27 (m, 3H), 6.95 (s, 1H), 6.60-6.56 (m, 2H), 6.09 (d, J=8.8 Hz, 1H), 4.40 (s, 1H), 3.77 (s, 3H), 3.72 (d, J=13.2 Hz, 2H), 3.52 (d, J=12.0 Hz, 2H), 3.18-3.10 (m, 2H), 2.89-2.83 (m, 5H), 2.24 (s, 3H), 1.74 (bs, 4H), 1.52 (bs, 2H), LC-MS C₃₂H₃₉N₆O₃ (M+H)⁺ calculated mass, 555.3084; found, 555.51.

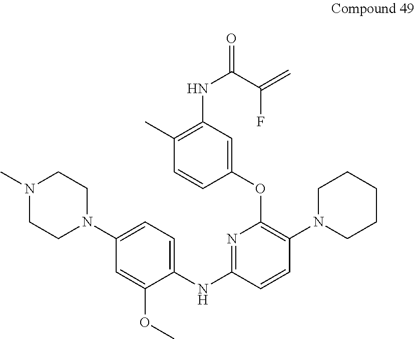 [SMILES: C=C(F)C(=O)Nc1cc(Oc2nc(Nc3ccc(N4CCN(C)CC4)cc3OC)ccc2N2CCCCC2)ccc1C]

### 2-fluoro-N-(5-((6-((2-methoxy-4-(4-methylpiperazin-1-yl)phenyl)amino)-3-(piperidin-1-yl)pyridin-2-yl)oxy)-2-methylphenyl)acrylamide

Prepared according to compound 47 using 2-fluoroacrylic acid in step e to afford the title compound (21 mg) as a gray solid. ¹H NMR (400 MHz, DMSO-d₆) δ 10.18 (s, 1H), 10.05 (s, 1H), 7.76 (s, 1H), 7.33 (d, J==8.4 Hz, 1H), 7.31-7.29 (m, 1H), 7.21 (s, 1H), 7.05 (d, J=8.0 Hz, 1H), 6.61-6.57 (m, 2H), 6.14 (d, J=8.8 Hz, 1H), 5.68 (dd, J=48.0, 3.6 Hz, 1H), 5.43 (dd, J=15.6, 3.6 Hz, 1H), 3.78 (s, 3H), 3.72 (d, J=12.8 Hz, 2H), 3.52 (d, J=12.0 Hz, 2H), 3.17-3.09 (m, 2H), 3.92 (d, J=12.8 Hz, 2H), 2.85 (s, 3H), 2.24 (s, 3H), 1.86 (bs, 4H), 1.59 (bs, 2H). LC-MS C₃₂H₄₀FN₆O₃ (M+H)⁺ calculated mass, 575.3146; found, 575.17.

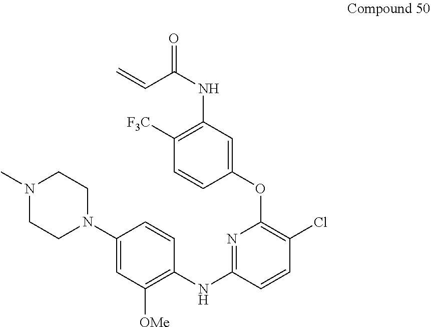 [SMILES: C=CC(=O)Nc1cc(Oc2nc(Nc3ccc(N4CCN(C)CC4)cc3OC)ccc2Cl)ccc1C(F)(F)F]

### N-(5-((3-chloro-6-((2-methoxy-4-(4-methylpiperazin-1-yl)phenyl)amino)pyridin-2-yl)oxy)-2-(trifluoromethyl)phenyl)acrylamide

Prepared according to example 1 (scheme 1) using 3-nitro-4-(trifluoromethyl)phenol step a to afford the title compound as a gray solid. ¹H NMR (400 MHz, DMSO-d₆) δ 9.82 (s, 1H), 9.73 (s, 1H), 8.33 (s, 1H), 7.79 (d, J=8.8 Hz, 1H), 7.67 (d, J=8.4 Hz, 1H), 7.37 (d, J=2.4 Hz, 1H), 7.28 (d, J=8.8 Hz, 1H), 7.22 (dd, J=8.4; 2.4 Hz, 1H), 6.66 (d, J=8.4 Hz, 1H), 662 (d, J=2.8 Hz, 1H), 6.54 (dd, J=16.8, 10.0 Hz, 1H), 6.24 (dd, J=16.8, 2.0 Hz, 1H), 6.13 (dd, J=8.8, 2.4 Hz, 1H), 5.78 (dd, J=10.0, 2.0 Hz, 1H), 3.78 (s, 3H), 3.73-3.69 (m, 2H), 3.53-3.50 (m, 2H), 3.17-3.10 (m, 2H), 2.91-2.88 (m, 2H), 2.86 (s, 3H). HRMS C₂₇H₂₈ClF₃N₅O₃ (M+H)⁺ calculated mass, 562.1833; found, 562.1862.

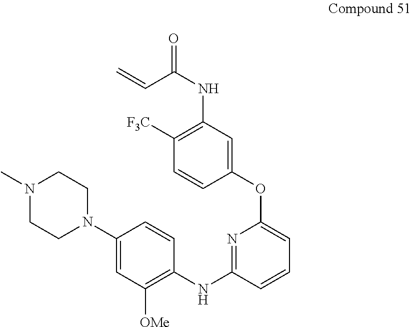 [SMILES: C=CC(=O)Nc1cc(Oc2cccc(Nc3ccc(N4CCN(C)CC4)cc3OC)n2)ccc1C(F)(F)F]

### N-(5-((6-((2-methoxy-4-(4-methylpiperazin-1-yl)phenyl)amino)pyridin-2-yl)oxy)-2-(trifluoro methyl)phenyl)acrylamide

In purification of compound 50 by reverse-phase HPLC, we obtained compound 51 at the same time. ¹H NMR (400 MHz, DMSO-d₆) 9.82 (s, 1H), 9.79 (s, 1H), 8.10 (s, 1H), 7.77 (d, J=8.8 Hz, 1H), 7.60 (t, J=8.0 Hz, 1H), 7.42 (d, J=8.8 Hz, 1H), 7.31 (d, J=2.4 Hz, 1H), 7.18 (dd, J=8.4, 2.4 Hz, 1H), 6.64 (d, J=2.4 Hz, 1H), 6.60 (d, J=8.0 Hz, 1H), 6.54 (dd, J=16.8, 10.0 Hz, 1H), 6.32 (d, J=7.6 Hz, 1H), 6.23 (dd, J=16.8, 2.0 Hz, 1H), 6.21 (dd, J=8.8, 2.4 Hz, 1H), 5.77 (dd, J=10.0, 2.0 Hz, 1H), 3.79 (s, 3H), 3.72 (d, J=13.6 Hz, 2H), 3.52 (d, J=12.0 Hz, 2H), 3.18-3.10 (m, 2H), 2.91-2.84 (m, 2H), 2.87 (s, 3H). HRMS C₂₇H₂₉F₃N₅O₃ (M+H)⁺ calculated mass, 528.2222; found, 528.2271.

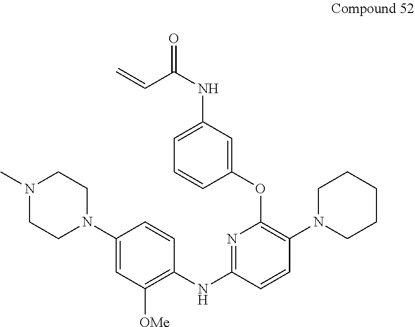 [SMILES: C=CC(=O)Nc1cccc(Oc2nc(Nc3ccc(N4CCN(C)CC4)cc3OC)ccc2N2CCCCC2)c1]

### N-(3-((6-((2-methoxy-4-(4-methylpiperazin-1-yl)phenyl)amino)-3-(piperidin-1-yl)pyridin-2-yl)oxy)phenyl)acrylamide

Prepared according to example 2 (scheme 2) using 3-nitrophenol in step a to afford the title compound as a brown solid. ¹H NMR (400 MHz, DMSO-d₆) 10.35 (s, 1H), 9.91 (s, 1H), 7.58 (d, J=8.4 Hz, 2H), 7.40 (t, J=8.4 Hz, 1H), 7.34 (d, J=8.4 Hz, 1H), 6.92 (d, J=8.4 Hz, 1H), 6.63 (d, J=8.4 Hz, 1H), 6.59 (s, 1H), 6.44 (dd, J=16.8, 10.0 Hz, 1H), 6.26 (dd, J=16.8, 2.0 Hz, 1H), 6.05 (d, J=8.8 Hz, 1H), 5.77 (dd, J=10.0, 2.0 Hz, 1H), 3.78 (s, 3H), 3.67 (d, J=13.2 Hz, 2H), 3.51 (d, J=12.0 Hz, 2H), 3.16-3.09 (m, 2H), 2.86 (s, 3H), 2.84-2.81 (m, 2H), 1.83 (bs, 4H), 1.58 (bs, 2H). HRMS C₃₁H₃₈N₆O₃ (M+H)⁺ calculated mass, 543.3084; found, 543.3133.

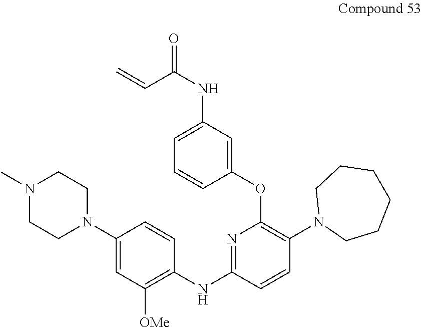 [SMILES: C=CC(=O)Nc1cccc(Oc2nc(Nc3ccc(N4CCN(C)CC4)cc3OC)ccc2N2CCCCCC2)c1]

### N-(3-((3-(azepan-1-yl)-6-((2-methoxy-4-(4-methylpiperazin-1-yl)phenyl)amino)pyridin-2-yl)oxy)phenyl)acrylamide

Prepared according to example 2 (scheme 2) using 3-nitrophenol in step a and azepane in step b to afford the title compound as a brown solid. The step b reaction condition: t-BuONa, Xantphos, Pd₂(dba)₃, toluene, 110° C., overnight. The step c reaction condition: Cs₂CO₃, DavePhos, Pd₂(dba)₃, 1,2-dimethoxyethane, 90° C., overnight. ¹H NMR (400 MHz, MeOD) δ 7.84 (s, 1H), 7.67 (d, J=8.0 Hz, 1H), 7.40 (t, J=8.4 Hz, 1H), 7.60 (s, 1H), 7.46 (t, J=8.0 Hz, 1H), 7.01 (d, J=8.4 Hz, 1H), 6.60 (bs, 1H), 8.44 (dd, J=16.8, 9.6 Hz, 1H), 6.37 (dd, J=16.8, 2.4 Hz, 1H), 6.13 (bs, 1H), 5.80 (dd, J=9.6, 2.4 Hz, 1H), 3.83 (bs, 4H), 3.59 (d, J=12.0 Hz, 2H), 3.25-3.19 (m, 2H), 2.97 (s, 3H), 2.13 (bs, 4H), 1.87 (bs, 4H). HRMS C₃₂H₄₁N₆O₃ (M+H)⁺ calculated mass, 557.3240; found, 557.3273.

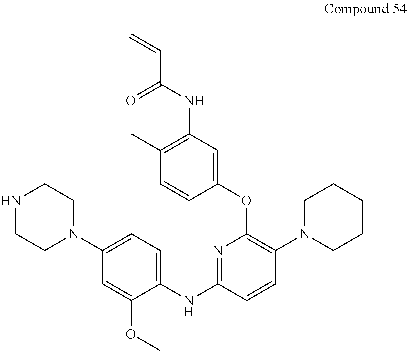 [SMILES: C=CC(=O)Nc1cc(Oc2nc(Nc3ccc(N4CCNCC4)cc3OC)ccc2N2CCCCC2)ccc1C]

### N-(5-((6-((2-methoxy-4-(piperazin-1-yl)phenyl)-3-(piperidin-1-yl)pyridin-2-yl)oxy)-2-methylphenyl)acrylamide

Prepared according to example 2 (scheme 2) using tert-butyl 4-(4-amino-3-methoxyphenyl)piperazine-1-carboxylate in step c to afford tert-butyl 4-(4-((6-(3-acrylamido-4-methylphenoxy)-5-(piperidin-1-yl)pyridin-2-yl)amino)-3-methoxyphen-yl)piperazine-1-carboxylate, and then the step of BOC de-protection accomplished through TFA to afford the title compound. ¹H NMR (400 MHz, DMSO-d₆) δ 9.54 (s, 1H), 8.81 (s, 1H), 7.51 (s, 1H), 7.32-7.28 (m, 2H), 6.95 (d, J=8.4 Hz, 1H), 6.62-6.56 (m, 3H), 6.23 (dd, J=16.8, 2.0 Hz, 1H), 6.26 (dd, J=16.8, 2.0 Hz, 1H), 6.07 (d, J=9.6 Hz, 1H), 5.75 (dd, J=10.0, 2.0 Hz, 1H), 3.77 (s, 3H), 3.45 (bs, 4H), 2.29 (s, 3H), 1.84 (bs, 4H), 1.58 (bs, 2H). HRMS C₃₁H₃₉N₆O₃ (M+H)⁺ calculated mass, 543.3084; found, 543.3109.

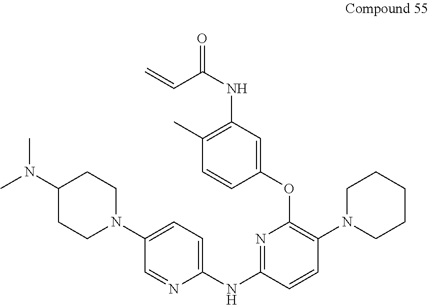 [SMILES: C=CC(=O)Nc1cc(Oc2nc(Nc3ccc(N4CCC(N(C)C)CC4)cn3)ccc2N2CCCCC2)ccc1C]

### N-(5-((6-((5-(4-(dimethylamino)piperidin-1-yl)pyridin-2-yl)amino-3-(piperidin-1-yl)pyridin-2-yl)oxy)-2-methylphenyl)acrylamide

### 5-(4-(dimethylamino)piperidin-1-yl)pyridin-2-amine

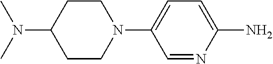 [SMILES: CN(C)C1CCN(c2ccc(N)nc2)CC1]

5-(4-(dimethylamino)piperidin-1-yl)pyridin-2-amine was prepared following the reference: J. Med. Chem., 2017, 60, 1892-1915.

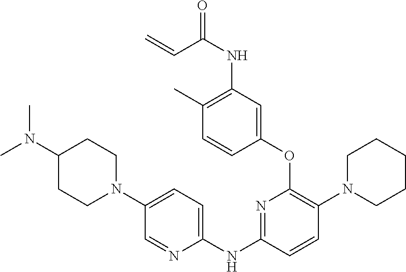 [SMILES: C=CC(=O)Nc1cc(Oc2nc(Nc3ccc(N4CCC(N(C)C)CC4)cn3)ccc2N2CCCCC2)ccc1C]

Prepared according to example 2 (scheme 2) using 5-(4-(dimethylamino)piperidin-1-yl)pyridin-2-amine in step c to afford the title compound as a yellow solid. ¹H NMR (400 MHz, DMSO-d₆) δ 9.79 (s, 1H), 9.76 (s, 1H), 7.72-7.64 (m, 2H), 7.46-7.45 (m, 1H), 7.43 (d, J=8.4 Hz, 1H), 7.22 (d, J==9.6 Hz, 1H), 7.04 (dd, J=8.0, 2.4 Hz, 1H), 6.92 (d, J=8.8 Hz, 1H), 6.55 (dd, 17.2, 10.0 Hz, 1H), 6.22 (dd, J===17.2, 2.0 Hz, 1H), 5.77 (dd, J=10.0, 2.0 Hz, 1H), 3.69 (d, J=8.8 Hz, 2H), 3.34-3.27 (m, 1H), 3.14 (bs, 4H), 2.80 (s, 3H), 2.78 (s, 3H), 2.66 (t, J=12.0 Hz, 2H), 2.31 (s, 3H), 2.07 (d, J=12.0 Hz, 2H), 1.75-1.64 (m, 6H), 1.58 (bs, 2H). HRMS C₃₄H₄₅N₆O₃ (M+H)⁺ calculated mass, 556.3400; found, 556.3466.

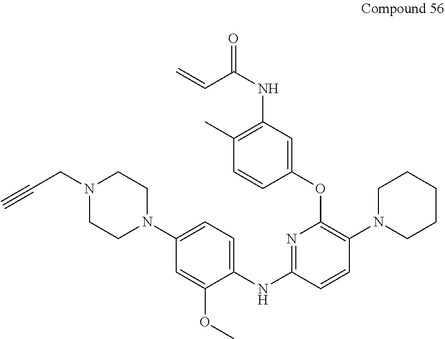 [SMILES: C#CCN1CCN(c2ccc(Nc3ccc(N4CCCCC4)c(Oc4ccc(C)c(NC(=O)C=C)c4)n3)c(OC)c2)CC1]

### N-(5-((6-((2-methoxy-4-(4-prop-2-yn-1-yl)piperazin-1-yl)phenyl)amino)-3-(piperidin-1-yl)pyridin-2-yl)oxy)-2-methylphenyl)acrylamide

Prepared according to the last step of preparation of compound 14, using compound 54 to replace compound 7 and 3-bromoprop-1-yne to replace (bromomethyl)cyclopropane to afford the title compound. ¹H NMR (400 MHz, DMSO-d₆) δ 9.45 (s, 1H), 7.47 (s, 1H), 7.40 (d, J=2.4 Hz, 1H), 7.32 (d, J=8.8 Hz, 1H), 7.29-7.23 (m, 2H), 6.83 (dd, J=8.4, 2.4 Hz, 1H), 6.58 (dd, J=17.2, 10.0 Hz, 1H), 6.52-6.50 (m, 2H), 6.22 (dd, J=17.2, 2.0 Hz, 1H), 6.02 (dd, J=8.8, 2.8 Hz, 1H), 5.73 (dd, J=10.0, 2.0 Hz, 1H), 3.75 (s, 3H), 3.19 (t, J=2.4 Hz, 1H), 3.03 (t, J=5.2 Hz, 2H), 2.90 (t, J=5.2 Hz, 2H), 2.58 (t, J=5.2 Hz, 2H), 2.27 (s, 3H), 1.64-1.59 (m, 4H), 1.51-1.45 (m, 2H). HRMS C₃₄H₄₅N₆O₃ (M+H)⁺ calculated mass, 581.3240; found, 581.3262.

In Vitro CatC Enzyme Assay

The CatC enzyme assay was performed in black 384-well plate. Firstly, rhCatC (440 nM) was activated by rhCatL (300 nM) in activation buffer (25 mM MES, 5 mM DTT, pH 6.0) at room temperature for 2 h and then activated rhCatC was diluted to 4.4 nM in assay buffer (25 mM MES, 50 mM NaCl, 5 mM DTT, 0.01% (v/v) Triton X100, pH 6.0), 14 μL activated rhCatC and 1 μL compound in 10% DMSO or 10% DMSO were incubated at room temperature for 3 h, and 5 μL substrate (Gly-Phe-AFC) was added to final concentration of 100 μM. After 60 min reaction, the product of the reaction was measured in the Enspire plate reader using Ex λ400 nm and Em λ505 nm for AFC. A standard irreversible CatC inhibitor 10 by AstraZeneca (J. Med. Chem. 2014, 57, 2357-2367) was used as an inhibitor control in the assay.

Mice/Rat In Vivo Studies

C57BL/6 mice (20-25 g) were randomly divided into 4 groups (n=10) and kept at 5 animals per cage. Compound 31 was administered orally twice daily at 6.7, 20, 60 mg kg-1 day-1 for 6 days. The matched vehicle controls (0.5% Methocel™, 0.1% Tween 80 in 100 mM citrate buffer, pH 3) were also administered twice daily for 6 days. Mice were weighed and the dose adjusted accordingly on each day. Sprague Dawley (280-300 g) were conducted by intraperitoneal injection twice daily at 1, 5, 20 mg kg-1 day-1 for 8 days. Blood was sampled through eye puncture at 14 h intervals after the end of the administration. Then mice were terminated and bone marrow cells were immediately extracted from femurs and tibias using ice cold RPMI 1640 medium. Blood cells and bone marrow cells were lysed according to the protocols previously described 8 with some modifications. Briefly, cell lysis buffer contained 50 mM Tris (pH 7.4), 750 mM NaCl, 1% (v/v) Triton X-100 and cell lysates were mildly treated by ultrasonic in ice. Following cell lysis, the supernatants were kept at −80° C. until NSP activity analyses.

Analysis of Downstream NSPs Activities.

The analysis was performed as previously described. Briefly, cell lysates were diluted into according protein concentration using different assay buffer. The assay buffer used were 25 mM MES, 50 mM NaCl, 5 mM DTT, pH 6.0 for Cat C, 50 mM Tris, 750 mM NaCl, pH 7.4 for NE and Pr3, and 50 mM Tris, 750 mM NaCl, 5 mM EDTA, pH 7.4 for CatG. Blood lysates were diluted into 5 fold higher than bone marrow lysates. Lysates were added to 384-well plates together with DMSO control or protease inhibitors (inhibitor 10 by AstraZeneca for CatC, avelestat for NE, sivelestat for Pr3, Cathepsin G inhibitor 1 for CatG). Synthetic peptide substrates (Gly-Phe-AFC(SMBiochemicals) for CatC, methoxysuccinyl-Ala-Ala-Pro-Val-AMC (Sigma) for NE, minobenzoyl-Val-Ala-Asp-Cys-Ala-Asp-Gin-ethylenediamine 2,4-dinitrophenyl (Peptide Synthetics) for Pr3, and N-succinyl-Ala-Ala-Pro-Phe-ONA (Sigma) for CatG) were added before plates were read at fluorescence or absorbance. Assay kinetics was monitored for up to 90 min. Data were analyzed using GraphPad Prism (GraphPad Software, Inc., San Diego, Calif., USA).

Induction of Pancreatitis.

The acute pancreatitis was induced by hourly intraperitoneal injections of caerulein (50 μg/kg/body weight) for up to 8 h, 36 C57BL/6 mice (20-25 g) were randomly divided into 4 groups (n=5). Compound 28 were administered orally twice daily at 20 mg/kg for 6 days. The matched vehicle controls (0.5% Methocel™, 0.1% Tween 80 in 100 mM citrate buffer, pH 3) were also administered twice daily for 6 days. On the 6th day, mice were induced pancreatitis after compound administration and after the last caerulein injection, mice were administered compound. The sham-treated group was performed with the same as vehicle controls except that saline was administered instead of caerulein. Mice were killed at 1 h after the last caerulein injection. Tissue was harvested immediately after sacrificing the animals. A portion of pancreas samples were frozen in liquid nitrogen for preparation of homogenate. For histology, pancreas was fixed in 4.5% formalin for paraffin embedding and stained with hematoxylin and eosin. Serum samples were prepared and stored at −20 C.

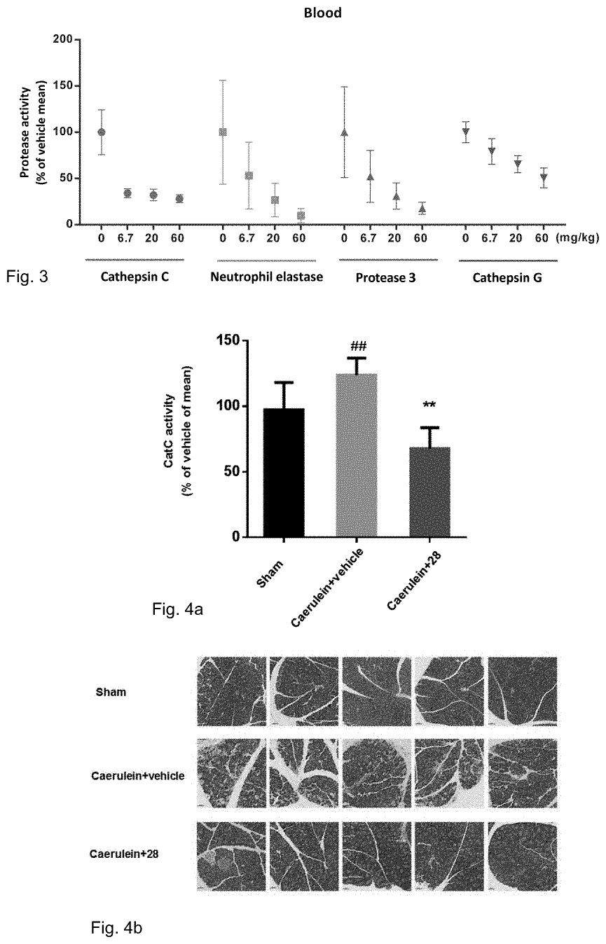

Compound 28 reduced the severity of acute pancreatitis. FIG. 4a shows CatC activity in homogenates prepared from mice pancreas samples. Compound 28 were administered orally twice daily at 20 mg/kg. The activity inhibition data of the compound-treated groups are shown as the percentage activity of a sham mean (set to 100% activity). Values are means±SEM. Statistical significance was considered: ^{##}p<0.05 versus Sham; ** p<0.05 versus caelurein+vehicle group. FIG. 4b shows pancreas histology representative photomicrographs. Hematoxylin and eosin staining of formalin-fixed sections of pancreas tissue. Scale bar: 50 μm.

TABLE 1
The Cathepsin C inhibitory activity of compounds; A: 3 nM < IC₅₀ < 300
nM, B: 30 uM > IC₅₀ > 300 nM

| Compound | | |
|---|---|---|
| Number | Structure | Cat C Enzy |
| 1 | 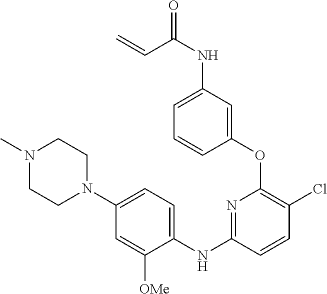 [SMILES: C=CC(=O)Nc1cccc(Oc2nc(Nc3ccc(N4CCN(C)CC4)cc3OC)ccc2Cl)c1] | B |
| 2 | 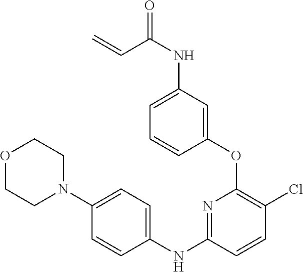 [SMILES: C=CC(=O)Nc1cccc(Oc2nc(Nc3ccc(N4CCOCC4)cc3)ccc2Cl)c1] | B |
| 3 | 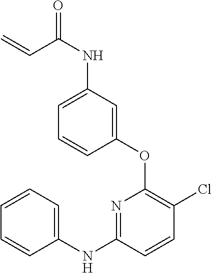 [SMILES: C=CC(=O)Nc1cccc(Oc2nc(Nc3ccccc3)ccc2Cl)c1] | B |
| 4 | 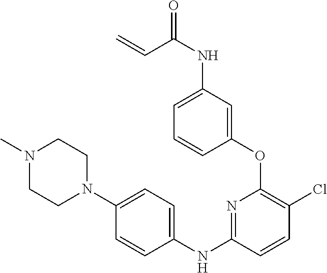 [SMILES: C=CC(=O)Nc1cccc(Oc2nc(Nc3ccc(N4CCN(C)CC4)cc3)ccc2Cl)c1] | B |
| 5 | 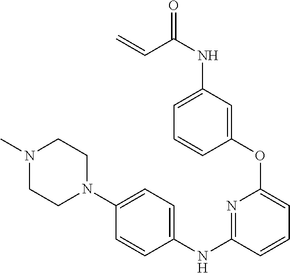 [SMILES: C=CC(=O)Nc1cccc(Oc2cccc(Nc3ccc(N4CCN(C)CC4)cc3)n2)c1] | B |
| 6 | 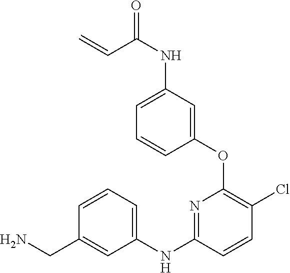 [SMILES: C=CC(=O)Nc1cccc(Oc2nc(Nc3cccc(CN)c3)ccc2Cl)c1] | B |
| 7 | 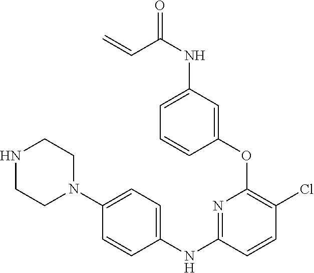 [SMILES: C=CC(=O)Nc1cccc(Oc2nc(Nc3ccc(N4CCNCC4)cc3)ccc2Cl)c1] | B |
| 8 | 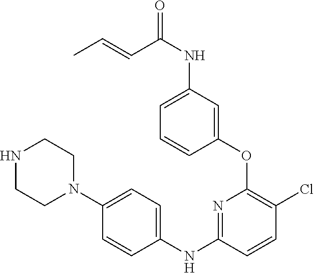 [SMILES: C/C=C/C(=O)Nc1cccc(Oc2nc(Nc3ccc(N4CCNCC4)cc3)ccc2Cl)c1] | B |
| 9 | 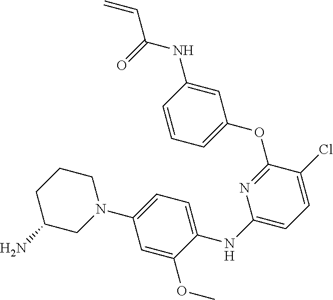 [SMILES: C=CC(=O)Nc1cccc(Oc2nc(Nc3ccc(N4CCC[C@@H](N)C4)cc3OC)ccc2Cl)c1] | B |
| 10 | 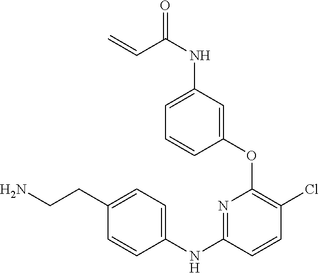 [SMILES: C=CC(=O)Nc1cccc(Oc2nc(Nc3ccc(CCN)cc3)ccc2Cl)c1] | B |
| 11 | 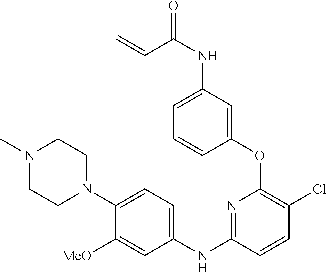 [SMILES: C=CC(=O)Nc1cccc(Oc2nc(Nc3ccc(N4CCN(C)CC4)c(OC)c3)ccc2Cl)c1] | B |
| 12 | 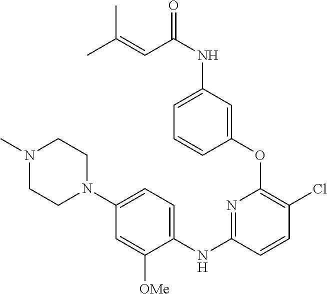 [SMILES: COc1cc(N2CCN(C)CC2)ccc1Nc1ccc(Cl)c(Oc2cccc(NC(=O)C=C(C)C)c2)n1] | B |
| 13 | 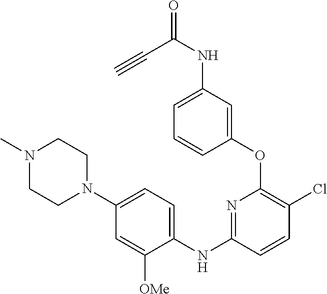 [SMILES: C#CC(=O)Nc1cccc(Oc2nc(Nc3ccc(N4CCN(C)CC4)cc3OC)ccc2Cl)c1] | B |
| 14 | 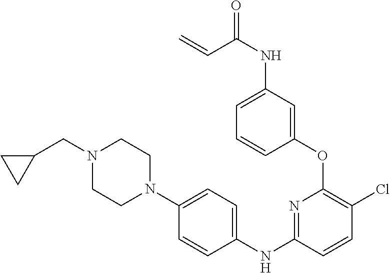 [SMILES: C=CC(=O)Nc1cccc(Oc2nc(Nc3ccc(N4CCN(CC5CC5)CC4)cc3)ccc2Cl)c1] | B |
| 15 | 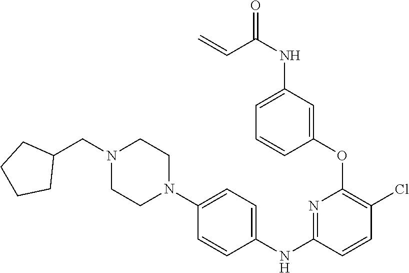 [SMILES: C=CC(=O)Nc1cccc(Oc2nc(Nc3ccc(N4CCN(CC5CCCC5)CC4)cc3)ccc2Cl)c1] | B |
| 16 | 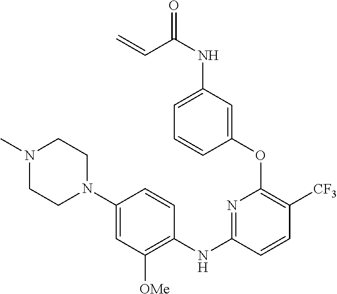 [SMILES: C=CC(=O)Nc1cccc(Oc2nc(Nc3ccc(N4CCN(C)CC4)cc3OC)ccc2C(F)(F)F)c1] | B |
| 17 | 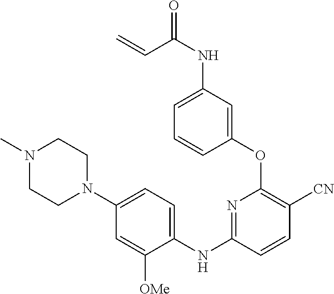 [SMILES: C=CC(=O)Nc1cccc(Oc2nc(Nc3ccc(N4CCN(C)CC4)cc3OC)ccc2C#N)c1] | B |
| 18 | 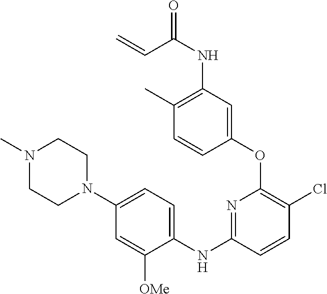 [SMILES: C=CC(=O)Nc1cc(Oc2nc(Nc3ccc(N4CCN(C)CC4)cc3OC)ccc2Cl)ccc1C] | B |
| 19 | 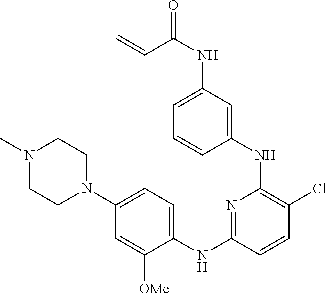 [SMILES: C=CC(=O)Nc1cccc(Nc2nc(Nc3ccc(N4CCN(C)CC4)cc3OC)ccc2Cl)c1] | B |
| 20 | 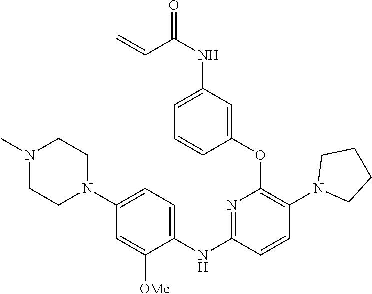 [SMILES: C=CC(=O)Nc1cccc(Oc2nc(Nc3ccc(N4CCN(C)CC4)cc3OC)ccc2N2CCCC2)c1] | A |
| 21 | 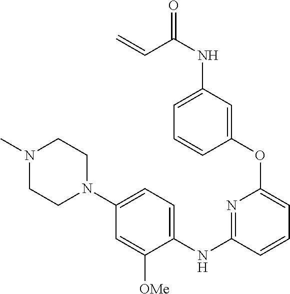 [SMILES: C=CC(=O)Nc1cccc(Oc2cccc(Nc3ccc(N4CCN(C)CC4)cc3OC)n2)c1] | B |
| 22 | 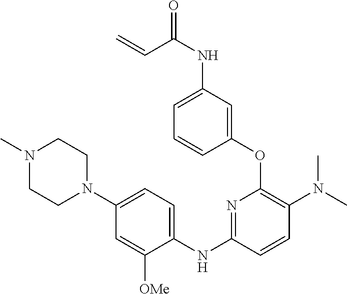 | B |
| 23 | 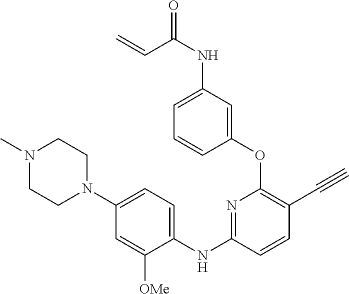 [SMILES: C#Cc1ccc(Nc2ccc(N3CCN(C)CC3)cc2OC)nc1Oc1cccc(NC(=O)C=C)c1] | B |
| 24 | 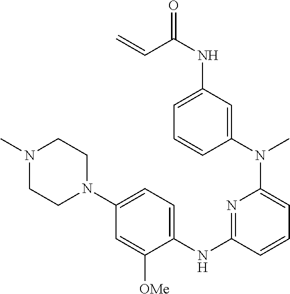 | B |
| 25 | 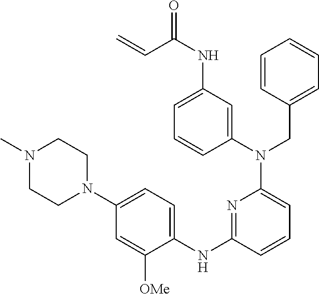 [SMILES: C=CC(=O)Nc1cccc(N(Cc2ccccc2)c2cccc(Nc3ccc(N4CCN(C)CC4)cc3OC)n2)c1] | B |
| 26 | 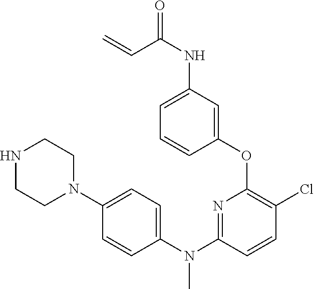 [SMILES: C=CC(=O)Nc1cccc(Oc2nc(N(C)c3ccc(N4CCNCC4)cc3)ccc2Cl)c1] | B |
| 27 | 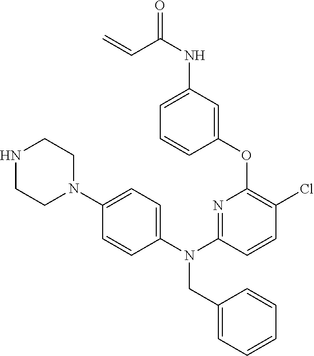 [SMILES: C=CC(=O)Nc1cccc(Oc2nc(N(Cc3ccccc3)c3ccc(N4CCNCC4)cc3)ccc2Cl)c1] | B |
| 28 | 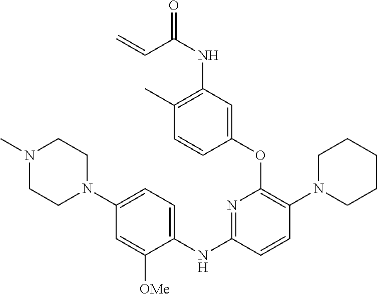 [SMILES: C=CC(=O)Nc1cc(Oc2nc(Nc3ccc(N4CCN(C)CC4)cc3OC)ccc2N2CCCCC2)ccc1C] | A |
| 29 | 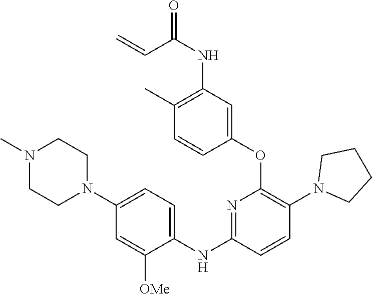 [SMILES: C=CC(=O)Nc1cc(Oc2nc(Nc3ccc(N4CCN(C)CC4)cc3OC)ccc2N2CCCC2)ccc1C] | A |
| 30 | 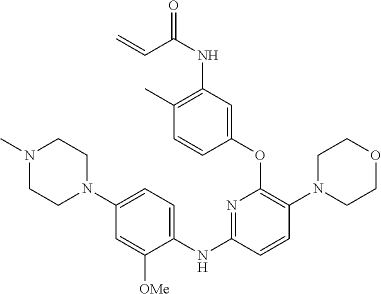 [SMILES: C=CC(=O)Nc1cc(Oc2nc(Nc3ccc(N4CCN(C)CC4)cc3OC)ccc2N2CCOCC2)ccc1C] | A |
| 31 | 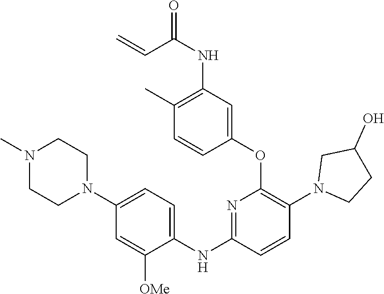 [SMILES: C=CC(=O)Nc1cc(Oc2nc(Nc3ccc(N4CCN(C)CC4)cc3OC)ccc2N2CCC(O)C2)ccc1C] | A |
| 32 | 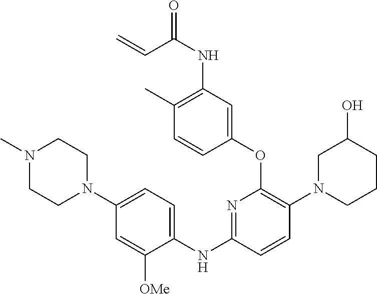 [SMILES: C=CC(=O)Nc1cc(Oc2nc(Nc3ccc(N4CCN(C)CC4)cc3OC)ccc2N2CCCC(O)C2)ccc1C] | A |
| 33 | 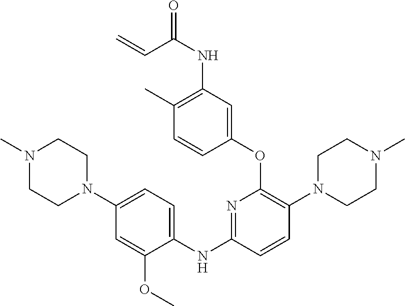 | A |
| 34 | 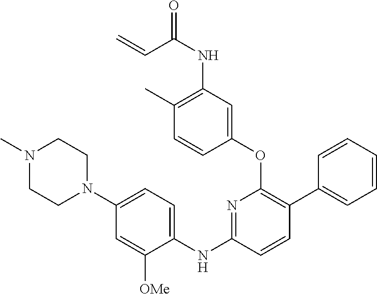 [SMILES: C=CC(=O)Nc1cc(Oc2nc(Nc3ccc(N4CCN(C)CC4)cc3OC)ccc2-c2ccccc2)ccc1C] | A |
| 35 | 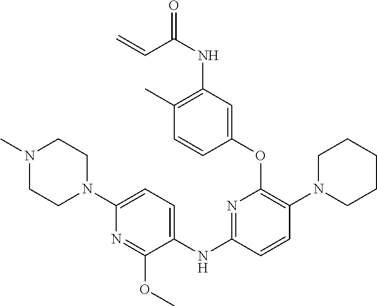 [SMILES: C=CC(=O)Nc1cc(Oc2nc(Nc3ccc(N4CCN(C)CC4)nc3OC)ccc2N2CCCCC2)ccc1C] | A |
| 36 | 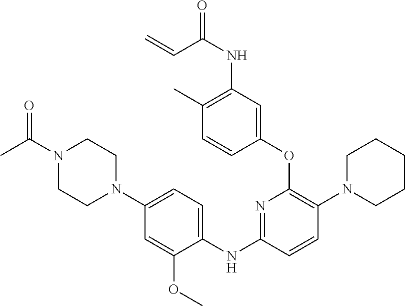 [SMILES: C=CC(=O)Nc1cc(Oc2nc(Nc3ccc(N4CCN(C(C)=O)CC4)cc3OC)ccc2N2CCCCC2)ccc1C] | A |
| 37 | 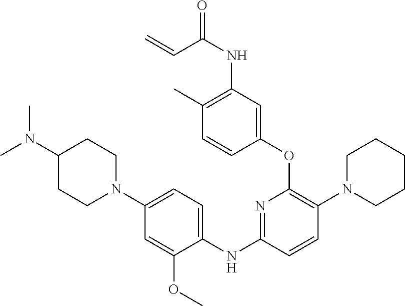 [SMILES: C=CC(=O)Nc1cc(Oc2nc(Nc3ccc(N4CCC(N(C)C)CC4)cc3OC)ccc2N2CCCCC2)ccc1C] | A |
| 38 | 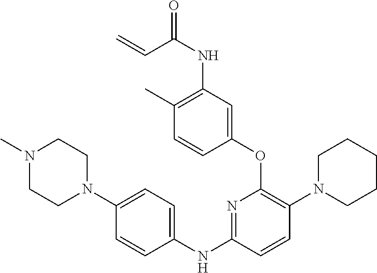 [SMILES: C=CC(=O)Nc1cc(Oc2nc(Nc3ccc(N4CCN(C)CC4)cc3)ccc2N2CCCCC2)ccc1C] | A |
| 39 | 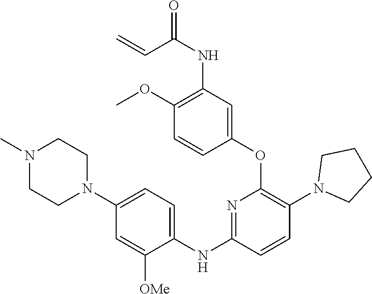 [SMILES: C=CC(=O)Nc1cc(Oc2nc(Nc3ccc(N4CCN(C)CC4)cc3OC)ccc2N2CCCC2)ccc1OC] | A |
| 40 | 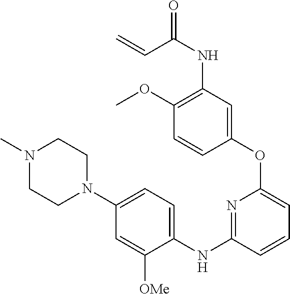 [SMILES: C=CC(=O)Nc1cc(Oc2cccc(Nc3ccc(N4CCN(C)CC4)cc3OC)n2)ccc1OC] | B |
| 41 | 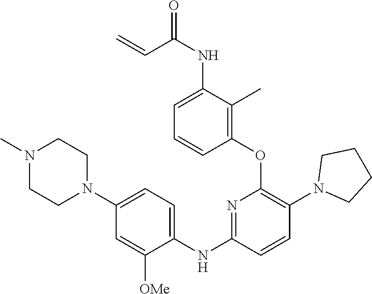 [SMILES: C=CC(=O)Nc1cccc(Oc2nc(Nc3ccc(N4CCN(C)CC4)cc3OC)ccc2N2CCCC2)c1C] | B |
| 42 | 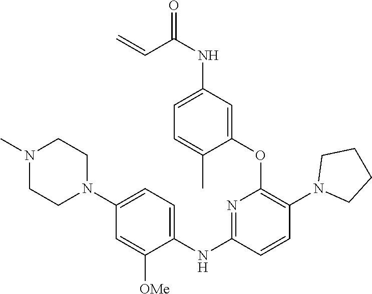 [SMILES: C=CC(=O)Nc1ccc(C)c(Oc2nc(Nc3ccc(N4CCN(C)CC4)cc3OC)ccc2N2CCCC2)c1] | B |
| 43 | 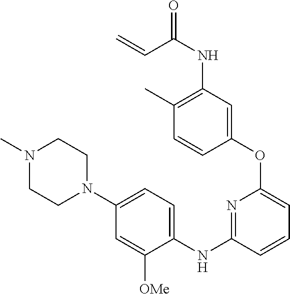 [SMILES: C=CC(=O)Nc1cc(Oc2cccc(Nc3ccc(N4CCN(C)CC4)cc3OC)n2)ccc1C] | A |
| 44 | 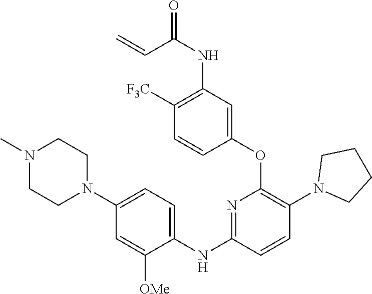 [SMILES: C=CC(=O)Nc1cc(Oc2nc(Nc3ccc(N4CCN(C)CC4)cc3OC)ccc2N2CCCC2)ccc1C(F)(F)F] | A |
| 45 | 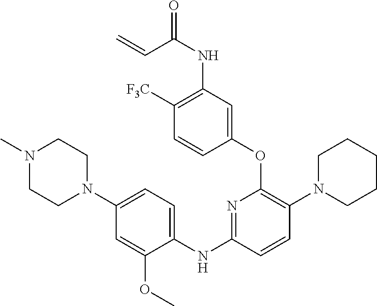 [SMILES: C=CC(=O)Nc1cc(Oc2nc(Nc3ccc(N4CCN(C)CC4)cc3OC)ccc2N2CCCCC2)ccc1C(F)(F)F] | A |
| 46 | 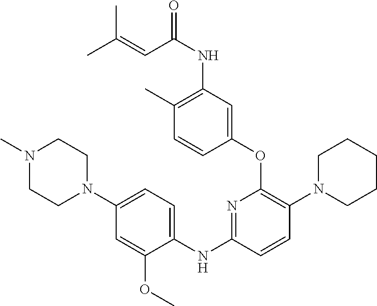 [SMILES: COc1cc(N2CCN(C)CC2)ccc1Nc1ccc(N2CCCCC2)c(Oc2ccc(C)c(NC(=O)C=C(C)C)c2)n1] | B |
| 47 | 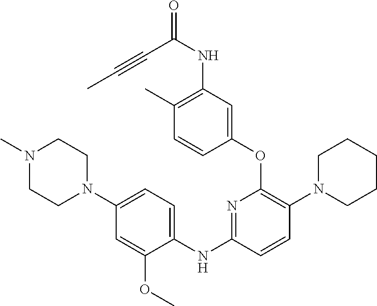 [SMILES: CC#CC(=O)Nc1cc(Oc2nc(Nc3ccc(N4CCN(C)CC4)cc3OC)ccc2N2CCCCC2)ccc1C] | B |
| 48 | 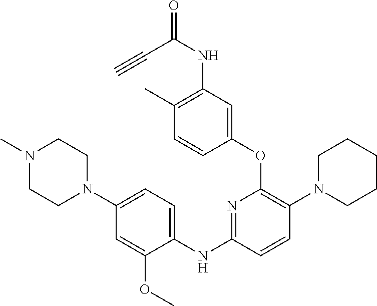 [SMILES: C#CC(=O)Nc1cc(Oc2nc(Nc3ccc(N4CCN(C)CC4)cc3OC)ccc2N2CCCCC2)ccc1C] | A |
| 49 | 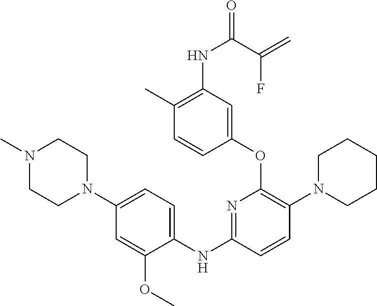 [SMILES: C=C(F)C(=O)Nc1cc(Oc2nc(Nc3ccc(N4CCN(C)CC4)cc3OC)ccc2N2CCCCC2)ccc1C] | B |
| 50 | 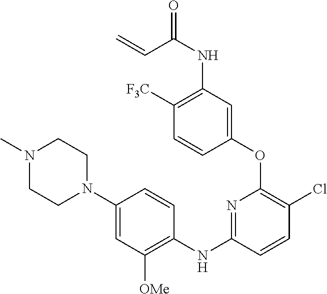 [SMILES: C=CC(=O)Nc1cc(Oc2nc(Nc3ccc(N4CCN(C)CC4)cc3OC)ccc2Cl)ccc1C(F)(F)F] | A |
| 51 | 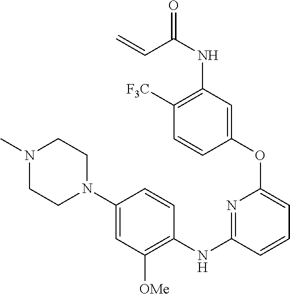 [SMILES: C=CC(=O)Nc1cc(Oc2cccc(Nc3ccc(N4CCN(C)CC4)cc3OC)n2)ccc1C(F)(F)F] | A |
| 52 | 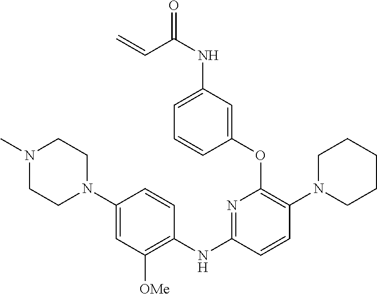 [SMILES: C=CC(=O)Nc1cccc(Oc2nc(Nc3ccc(N4CCN(C)CC4)cc3OC)ccc2N2CCCCC2)c1] | A |
| 53 | 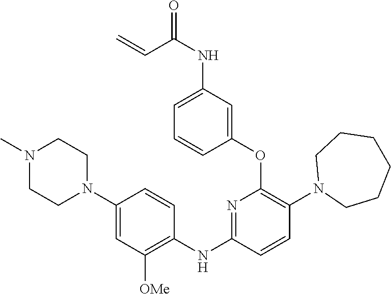 [SMILES: C=CC(=O)Nc1cccc(Oc2nc(Nc3ccc(N4CCN(C)CC4)cc3OC)ccc2N2CCCCCC2)c1] | A |
| 54 | 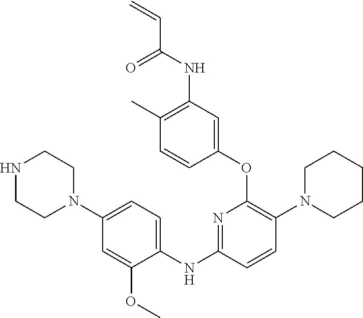 [SMILES: C=CC(=O)Nc1cc(Oc2nc(Nc3ccc(N4CCNCC4)cc3OC)ccc2N2CCCCC2)ccc1C] | A |
| 55 | 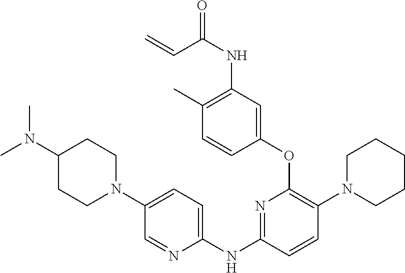 [SMILES: C=CC(=O)Nc1cc(Oc2nc(Nc3ccc(N4CCC(N(C)C)CC4)cn3)ccc2N2CCCCC2)ccc1C] | B |
| 56 | 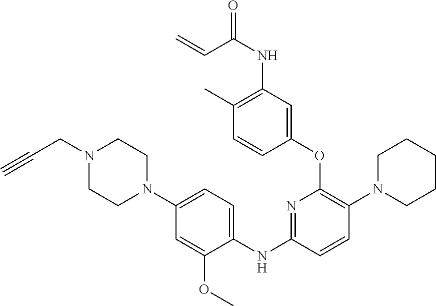 [SMILES: C#CCN1CCN(c2ccc(Nc3ccc(N4CCCCC4)c(Oc4ccc(C)c(NC(=O)C=C)c4)n3)c(OC)c2)CC1] | A |

TABLE 2
Preferred active inhibitors wherein Ar is substituted with a substituent
which forms a salt bridge with cathepsin C, particularly a cyclic or chain
amine, wherein the nitrogen atom of the amine forms a salt bridge with
cathepsin C, which helps maintain cathepsin C inhibitory activity.

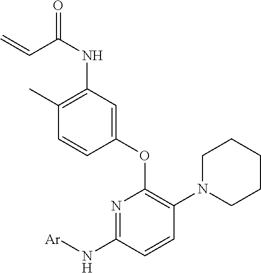

| Reg. No. | Structure Ar = |
|---|---|
| 28 | 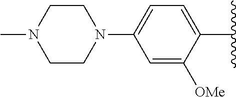 [SMILES: *c1ccc(N2CCN(C)CC2)cc1OC] |
| 35 | 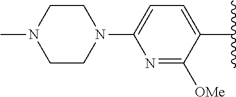 [SMILES: *c1ccc(N2CCN(C)CC2)nc1OC] |
| 38 | 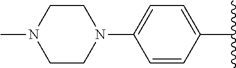 [SMILES: *c1ccc(N2CCN(C)CC2)cc1] |
| 36 | 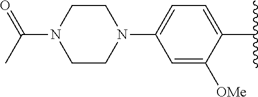 [SMILES: *c1ccc(N2CCN(C(C)=O)CC2)cc1OC] |
| 37 | 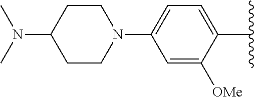 [SMILES: *c1ccc(N2CCC(N(C)C)CC2)cc1OC] |

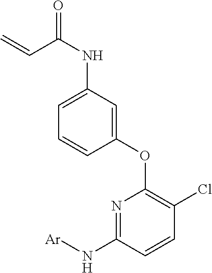

| Reg. No. | Structure Ar = |
|---|---|
| 1 | 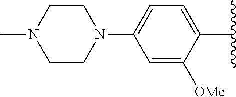 [SMILES: *c1ccc(N2CCN(C)CC2)cc1OC] |
| 11 | 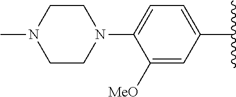 [SMILES: *c1ccc(N2CCN(C)CC2)c(OC)c1] |
| 4 | 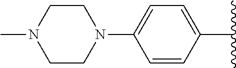 [SMILES: *c1ccc(N2CCN(C)CC2)cc1] |
| 7 | 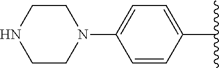 [SMILES: *c1ccc(N2CCNCC2)cc1] |
| 2 | 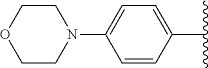 [SMILES: *c1ccc(N2CCOCC2)cc1] |
| 3 | 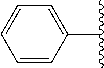 [SMILES: *c1ccccc1] |
| 6 | 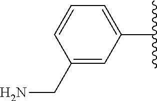 [SMILES: *c1cccc(CN)c1] |
| 10 | 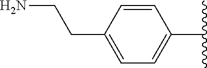 [SMILES: *c1ccc(CCN)cc1] |
| 9 | 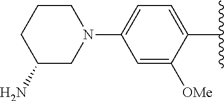 [SMILES: *c1ccc(N2CCC[C@@H](N)C2)cc1OC] |
| 14 | 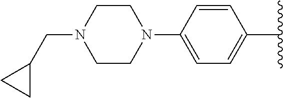 [SMILES: *c1ccc(N2CCN(CC3CC3)CC2)cc1] |
| 15 | 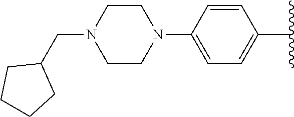 [SMILES: *c1ccc(N2CCN(CC3CCCC3)CC2)cc1] |

TABLE 3
Second Generation Cathepsin C inhibitors; G2 compounds shown provide
Cathepsin C inhibitory activity 3 nM < IC₅₀ ≤ 30 mM

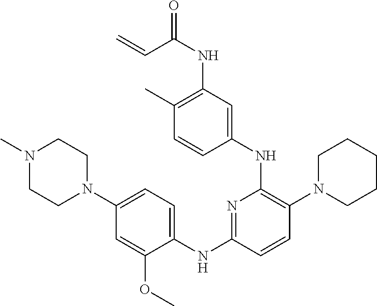 [SMILES: C=CC(=O)Nc1cc(Nc2nc(Nc3ccc(N4CCN(C)CC4)cc3OC)ccc2N2CCCCC2)ccc1C]

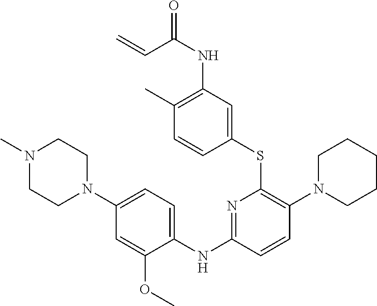 [SMILES: C=CC(=O)Nc1cc(Sc2nc(Nc3ccc(N4CCN(C)CC4)cc3OC)ccc2N2CCCCC2)ccc1C]

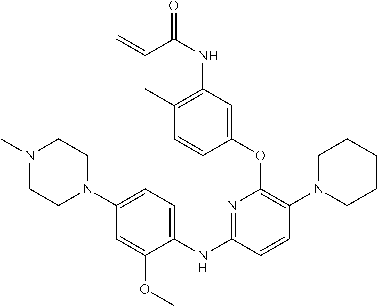 [SMILES: C=CC(=O)Nc1cc(Oc2nc(Nc3ccc(N4CCN(C)CC4)cc3OC)ccc2N2CCCCC2)ccc1C]

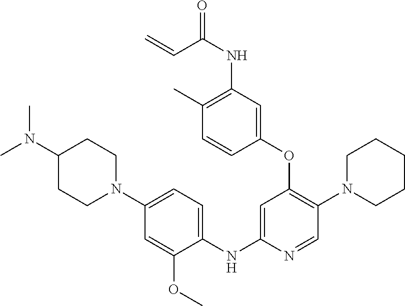 [SMILES: C=CC(=O)Nc1cc(Oc2cc(Nc3ccc(N4CCC(N(C)C)CC4)cc3OC)ncc2N2CCCCC2)ccc1C]

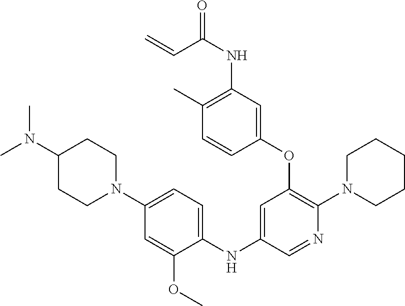 [SMILES: C=CC(=O)Nc1cc(Oc2cc(Nc3ccc(N4CCC(N(C)C)CC4)cc3OC)cnc2N2CCCCC2)ccc1C]

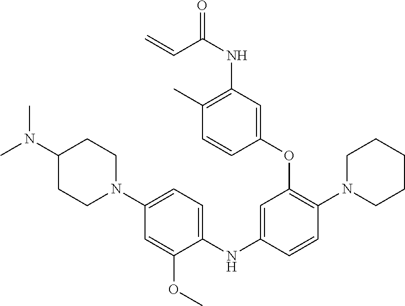 [SMILES: C=CC(=O)Nc1cc(Oc2cc(Nc3ccc(N4CCC(N(C)C)CC4)cc3OC)ccc2N2CCCCC2)ccc1C]

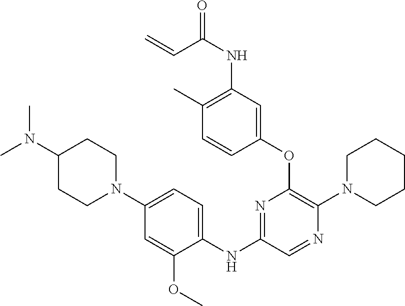 [SMILES: C=CC(=O)Nc1cc(Oc2nc(Nc3ccc(N4CCC(N(C)C)CC4)cc3OC)cnc2N2CCCCC2)ccc1C]

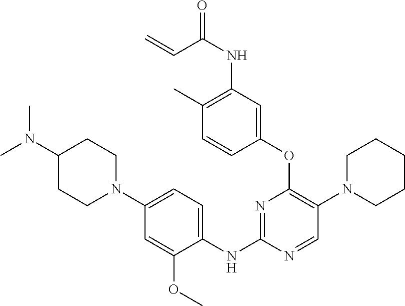 [SMILES: C=CC(=O)Nc1cc(Oc2nc(Nc3ccc(N4CCC(N(C)C)CC4)cc3OC)ncc2N2CCCCC2)ccc1C]

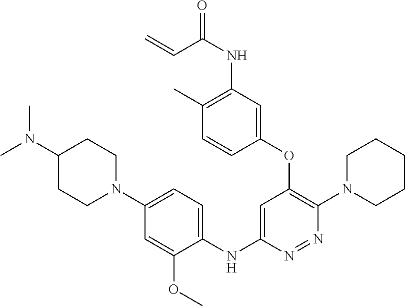 [SMILES: C=CC(=O)Nc1cc(Oc2cc(Nc3ccc(N4CCC(N(C)C)CC4)cc3OC)nnc2N2CCCCC2)ccc1C]

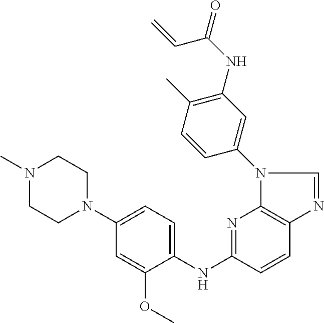 [SMILES: C=CC(=O)Nc1cc(-n2cnc3ccc(Nc4ccc(N5CCN(C)CC5)cc4OC)nc32)ccc1C]

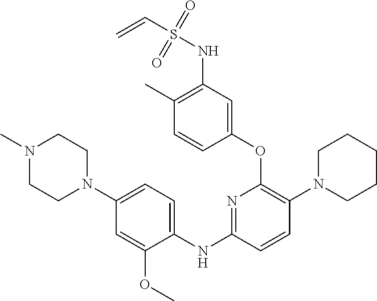 [SMILES: C=CS(=O)(=O)Nc1cc(Oc2nc(Nc3ccc(N4CCN(C)CC4)cc3OC)ccc2N2CCCCC2)ccc1C]

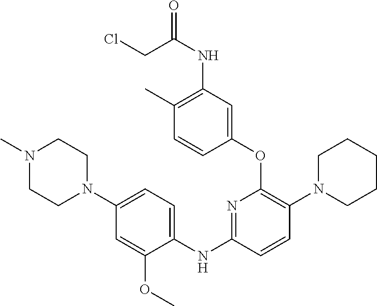 [SMILES: COc1cc(N2CCN(C)CC2)ccc1Nc1ccc(N2CCCCC2)c(Oc2ccc(C)c(NC(=O)CCl)c2)n1]

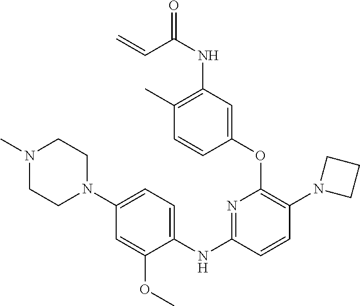 [SMILES: C=CC(=O)Nc1cc(Oc2nc(Nc3ccc(N4CCN(C)CC4)cc3OC)ccc2N2CCC2)ccc1C]

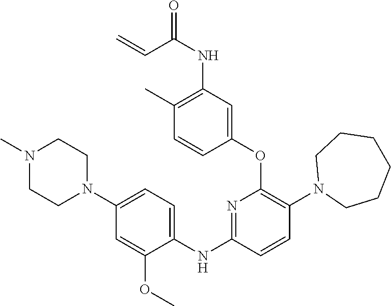 [SMILES: C=CC(=O)Nc1cc(Oc2nc(Nc3ccc(N4CCN(C)CC4)cc3OC)ccc2N2CCCCCC2)ccc1C]

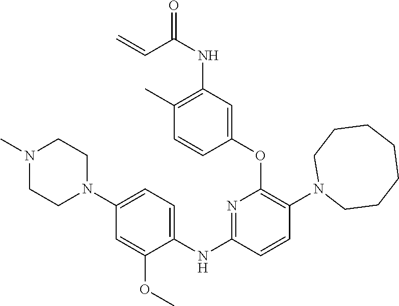 [SMILES: C=CC(=O)Nc1cc(Oc2nc(Nc3ccc(N4CCN(C)CC4)cc3OC)ccc2N2CCCCCCC2)ccc1C]

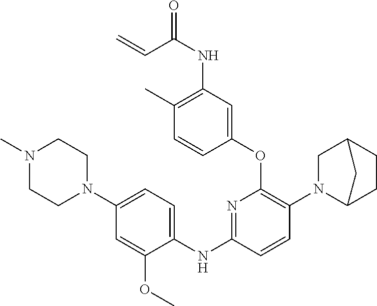 [SMILES: C=CC(=O)Nc1cc(Oc2nc(Nc3ccc(N4CCN(C)CC4)cc3OC)ccc2N2CC3CCC2C3)ccc1C]

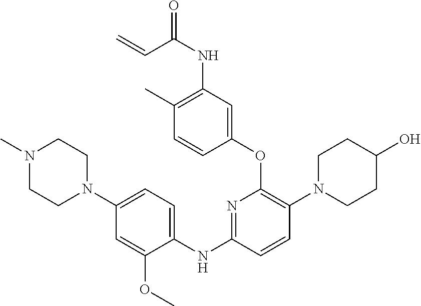 [SMILES: C=CC(=O)Nc1cc(Oc2nc(Nc3ccc(N4CCN(C)CC4)cc3OC)ccc2N2CCC(O)CC2)ccc1C]

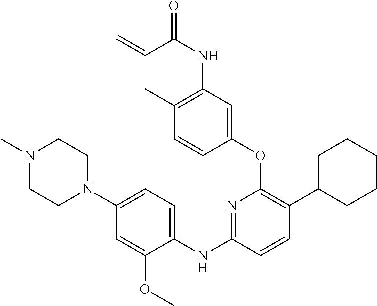 [SMILES: C=CC(=O)Nc1cc(Oc2nc(Nc3ccc(N4CCN(C)CC4)cc3OC)ccc2C2CCCCC2)ccc1C]

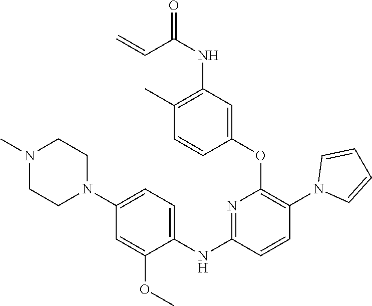 [SMILES: C=CC(=O)Nc1cc(Oc2nc(Nc3ccc(N4CCN(C)CC4)cc3OC)ccc2-n2cccc2)ccc1C]

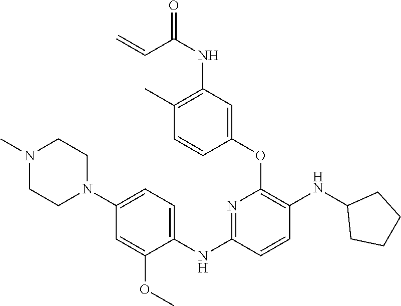 [SMILES: C=CC(=O)Nc1cc(Oc2nc(Nc3ccc(N4CCN(C)CC4)cc3OC)ccc2NC2CCCC2)ccc1C]

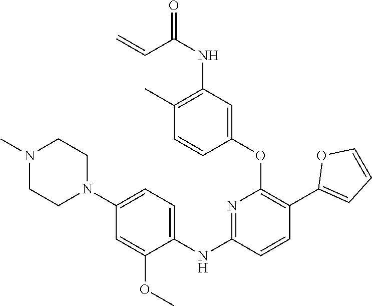 [SMILES: C=CC(=O)Nc1cc(Oc2nc(Nc3ccc(N4CCN(C)CC4)cc3OC)ccc2-c2ccco2)ccc1C]

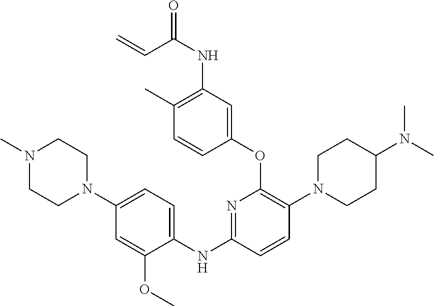

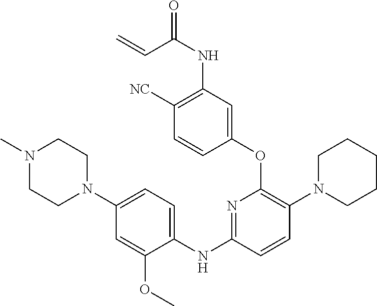 [SMILES: C=CC(=O)Nc1cc(Oc2nc(Nc3ccc(N4CCN(C)CC4)cc3OC)ccc2N2CCCCC2)ccc1C#N]

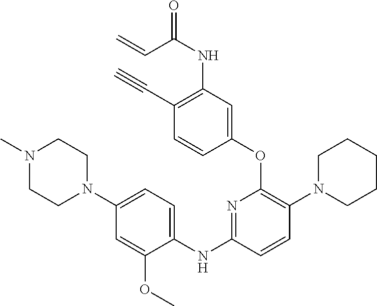 [SMILES: C#Cc1ccc(Oc2nc(Nc3ccc(N4CCN(C)CC4)cc3OC)ccc2N2CCCCC2)cc1NC(=O)C=C]

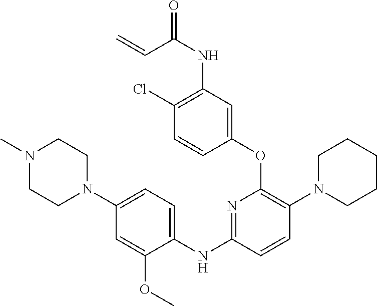 [SMILES: C=CC(=O)Nc1cc(Oc2nc(Nc3ccc(N4CCN(C)CC4)cc3OC)ccc2N2CCCCC2)ccc1Cl]

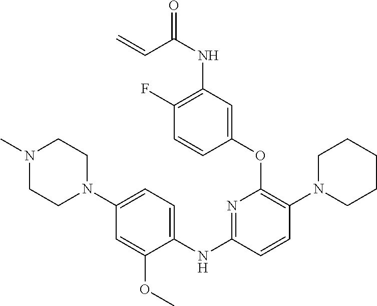 [SMILES: C=CC(=O)Nc1cc(Oc2nc(Nc3ccc(N4CCN(C)CC4)cc3OC)ccc2N2CCCCC2)ccc1F]

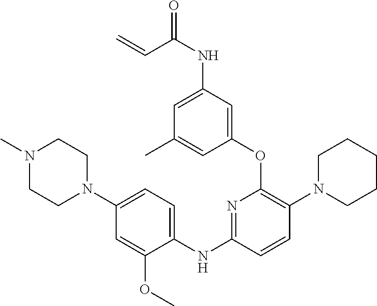 [SMILES: C=CC(=O)Nc1cc(C)cc(Oc2nc(Nc3ccc(N4CCN(C)CC4)cc3OC)ccc2N2CCCCC2)c1]

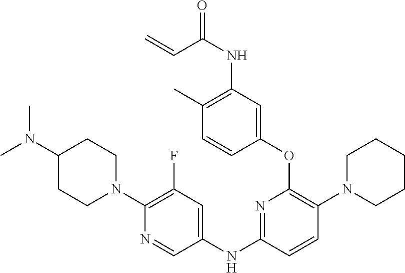 [SMILES: C=CC(=O)Nc1cc(Oc2nc(Nc3cnc(N4CCC(N(C)C)CC4)c(F)c3)ccc2N2CCCCC2)ccc1C]

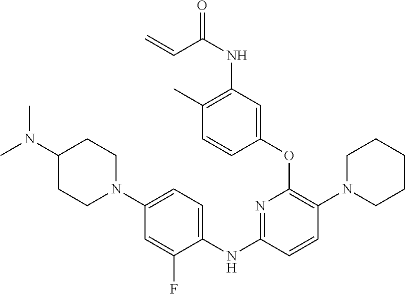 [SMILES: C=CC(=O)Nc1cc(Oc2nc(Nc3ccc(N4CCC(N(C)C)CC4)cc3F)ccc2N2CCCCC2)ccc1C]

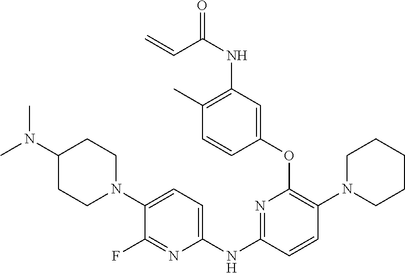 [SMILES: C=CC(=O)Nc1cc(Oc2nc(Nc3ccc(N4CCC(N(C)C)CC4)c(F)n3)ccc2N2CCCCC2)ccc1C]

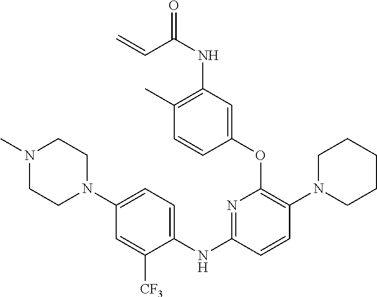 [SMILES: C=CC(=O)Nc1cc(Oc2nc(Nc3ccc(N4CCN(C)CC4)cc3C(F)(F)F)ccc2N2CCCCC2)ccc1C]

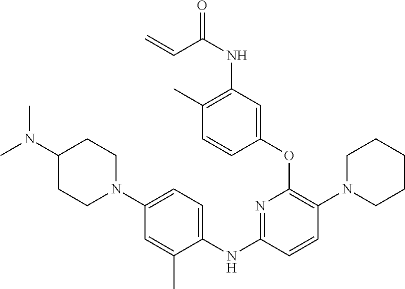 [SMILES: C=CC(=O)Nc1cc(Oc2nc(Nc3ccc(N4CCC(N(C)C)CC4)cc3C)ccc2N2CCCCC2)ccc1C]

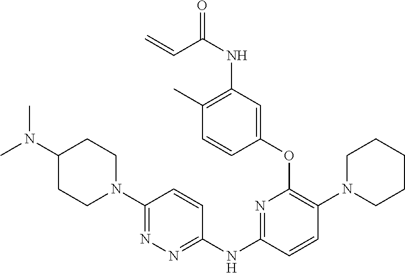 [SMILES: C=CC(=O)Nc1cc(Oc2nc(Nc3ccc(N4CCC(N(C)C)CC4)nn3)ccc2N2CCCCC2)ccc1C]

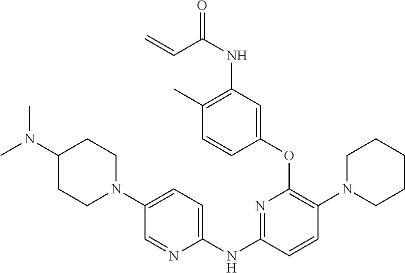 [SMILES: C=CC(=O)Nc1cc(Oc2nc(Nc3ccc(N4CCC(N(C)C)CC4)cn3)ccc2N2CCCCC2)ccc1C]

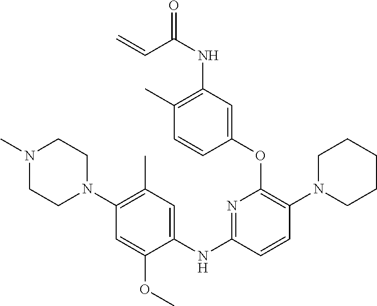 [SMILES: C=CC(=O)Nc1cc(Oc2nc(Nc3cc(C)c(N4CCN(C)CC4)cc3OC)ccc2N2CCCCC2)ccc1C]

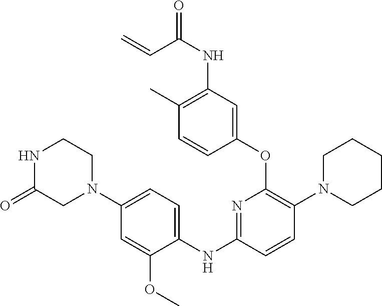 [SMILES: C=CC(=O)Nc1cc(Oc2nc(Nc3ccc(N4CCNC(=O)C4)cc3OC)ccc2N2CCCCC2)ccc1C]

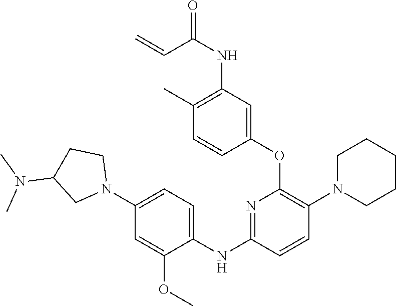 [SMILES: C=CC(=O)Nc1cc(Oc2nc(Nc3ccc(N4CCC(N(C)C)C4)cc3OC)ccc2N2CCCCC2)ccc1C]

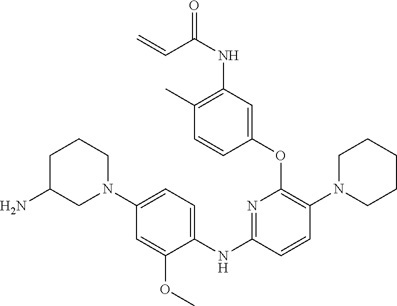 [SMILES: C=CC(=O)Nc1cc(Oc2nc(Nc3ccc(N4CCCC(N)C4)cc3OC)ccc2N2CCCCC2)ccc1C]

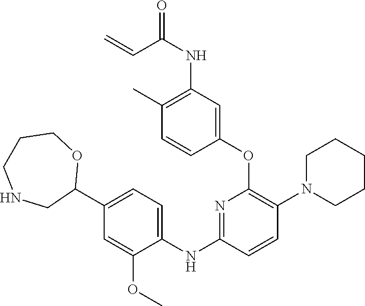 [SMILES: C=CC(=O)Nc1cc(Oc2nc(Nc3ccc(C4CNCCCO4)cc3OC)ccc2N2CCCCC2)ccc1C]

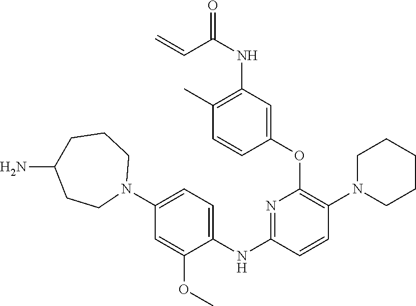 [SMILES: C=CC(=O)Nc1cc(Oc2nc(Nc3ccc(N4CCCC(N)CC4)cc3OC)ccc2N2CCCCC2)ccc1C]

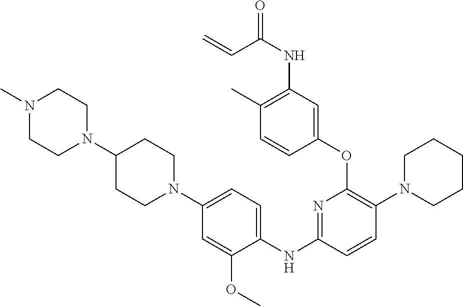 [SMILES: C=CC(=O)Nc1cc(Oc2nc(Nc3ccc(N4CCC(N5CCN(C)CC5)CC4)cc3OC)ccc2N2CCCCC2)ccc1C]

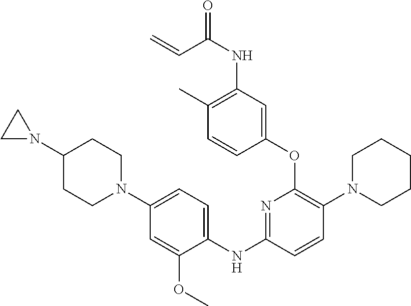 [SMILES: C=CC(=O)Nc1cc(Oc2nc(Nc3ccc(N4CCC(N5CC5)CC4)cc3OC)ccc2N2CCCCC2)ccc1C]

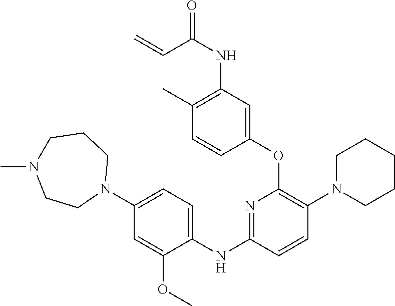 [SMILES: C=CC(=O)Nc1cc(Oc2nc(Nc3ccc(N4CCCN(C)CC4)cc3OC)ccc2N2CCCCC2)ccc1C]

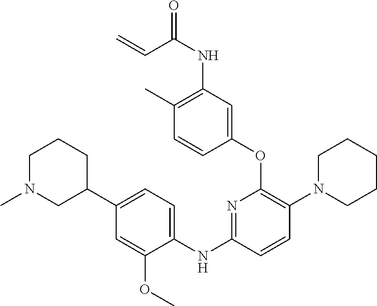 [SMILES: C=CC(=O)Nc1cc(Oc2nc(Nc3ccc(C4CCCN(C)C4)cc3OC)ccc2N2CCCCC2)ccc1C]

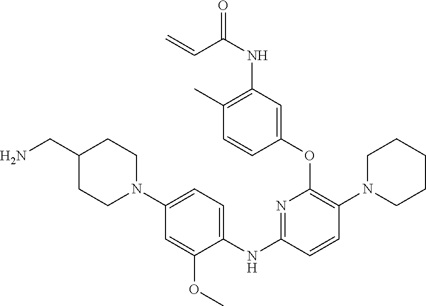 [SMILES: C=CC(=O)Nc1cc(Oc2nc(Nc3ccc(N4CCC(CN)CC4)cc3OC)ccc2N2CCCCC2)ccc1C]

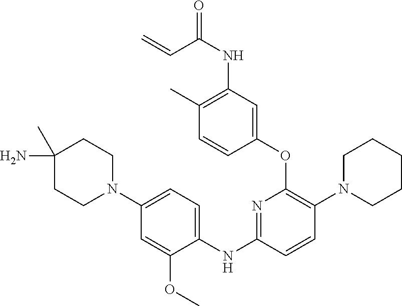 [SMILES: C=CC(=O)Nc1cc(Oc2nc(Nc3ccc(N4CCC(C)(N)CC4)cc3OC)ccc2N2CCCCC2)ccc1C]

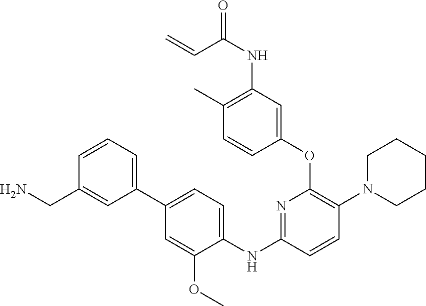 [SMILES: C=CC(=O)Nc1cc(Oc2nc(Nc3ccc(-c4cccc(CN)c4)cc3OC)ccc2N2CCCCC2)ccc1C]

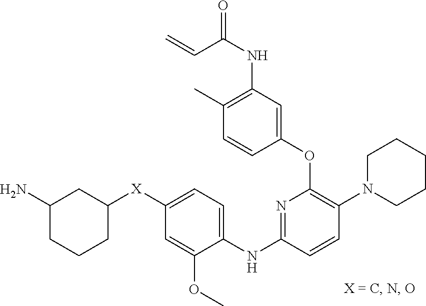

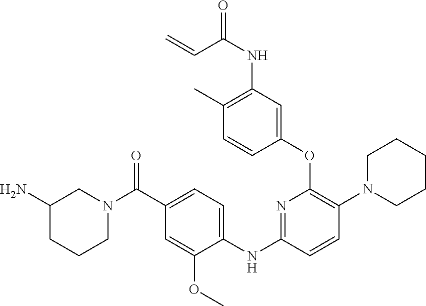 [SMILES: C=CC(=O)Nc1cc(Oc2nc(Nc3ccc(C(=O)N4CCCC(N)C4)cc3OC)ccc2N2CCCCC2)ccc1C]

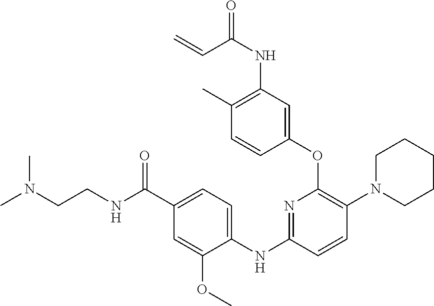 [SMILES: C=CC(=O)Nc1cc(Oc2nc(Nc3ccc(C(=O)NCCN(C)C)cc3OC)ccc2N2CCCCC2)ccc1C]

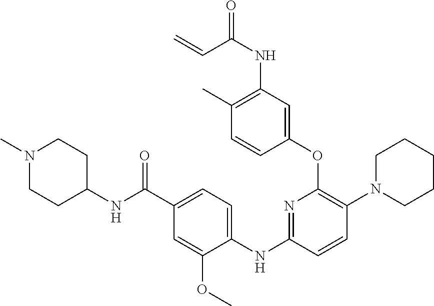 [SMILES: C=CC(=O)Nc1cc(Oc2nc(Nc3ccc(C(=O)NC4CCN(C)CC4)cc3OC)ccc2N2CCCCC2)ccc1C]

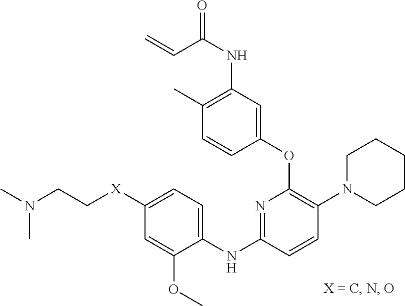

The examples herein are provided by way of illustration only and not by way of limitation. Those skilled in the art will readily recognize a variety of noncritical parameters that could be changed or modified to yield essentially similar results.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound of formula: 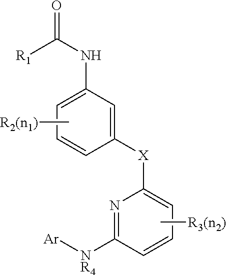
wherein:
X is O or S;
Ar is a substituted or unsubstituted 5-6 membered aryl or heteroaryl;
R1 is substituted or unsubstituted ethenyl or ethynyl;
R2 and R3 are independently optionally substituted, optionally hetero-, optionally cyclic C1-C18 hydrocarbyl, or optionally substituted heteroatom;
R4 is independently H, optionally substituted, optionally hetero-, optionally cyclic C1-C18 hydrocarbyl, or optionally substituted heteroatom, and
each of n1 and n2 is independently 0, 1, 2 or 3;
or a pharmaceutically acceptable salt, hydrate or stereoisomer the compound.

2. The compound of claim 1 wherein:
X is O.
Ar is substituted phenyl;
R1 is ethenyl, ethynyl, propenyl, 2-methylpropenyl, or propyne, each optionally fluorinated;
R2 and R3 are independently optionally substituted, optionally hetero-, optionally cyclic C1-C18 hydrocarbyl, or optionally substituted heteroatom;
R4 is independently H, optionally substituted C1-C4 alkyl, alkenyl or alkynyl or CN, each optionally fluorinated.

3. The compound of claim 1 wherein:
X is O;
Ar is substituted phenyl;
R1 is ethenyl;
R2 and R3 are independently optionally substituted, optionally hetero-, optionally cyclic C1-C18 hydrocarbyl, or optionally substituted heteroatom; and
R4 is H.

4. The compound of claim 1 wherein:
X is O;
Ar is substituted phenyl;
R1 is ethenyl;
R2 is halogen, CN, optionally substituted C1-C4 alkyl, alkenyl, alkynyl or alkyloxy, each optionally fluorinated;
R3 is optionally substituted, optionally hetero-, optionally cyclic C1-C18 hydrocarbyl, or optionally substituted heteroatom;
R4 is H;
n1 is 0 or 1; and
n2 is 1.

5. The compound of claim 1 wherein:
X is O;
Ar is substituted phenyl;
R1 is ethenyl;
R2 is halogen, CN, optionally substituted C1-C4 alkyl, alkenyl, alkenyl or alkyloxy, each optionally fluorinated, and positioned para to X,
R3 is optionally substituted, optionally hetero-, optionally cyclic C1-C18 hydrocarbyl, or optionally substituted heteroatom, and positioned para to NR4;
R4 is H;
n1 is 0 or 1; and
n2 is 1.

6. The compound of claim 1 wherein:
X is O;
Ar is substituted phenyl;
R1 is ethenyl;
R2 is halogen, CN, optionally substituted C1-C4 alkyl, alkenyl, alkynyl or alkyloxy, each optionally fluorinated, and positioned para to X,
R3 is substituted N, and positioned para to NR4;
R4 is H;
n1 is 1; and
n2 is 1.

7. The compound of claim 1 wherein:
X is O;
Ar is substituted phenyl;
R1 is ethenyl;
R2 is methyl, and positioned para to X,
R3 is N-piperidinyl, and positioned para to NR4;
R4 is H;
n1 is 1; and
n2 is 1.

8. The compound of claim 1 wherein: Ar is substituted with a substituent which forms a salt bridge with cathepsin C, particularly a cyclic or chain amine, wherein the nitrogen atom of the amine forms a salt bridge with cathepsin C.

9. The compound of claim 1 wherein: Ar comprises a structure selected from:

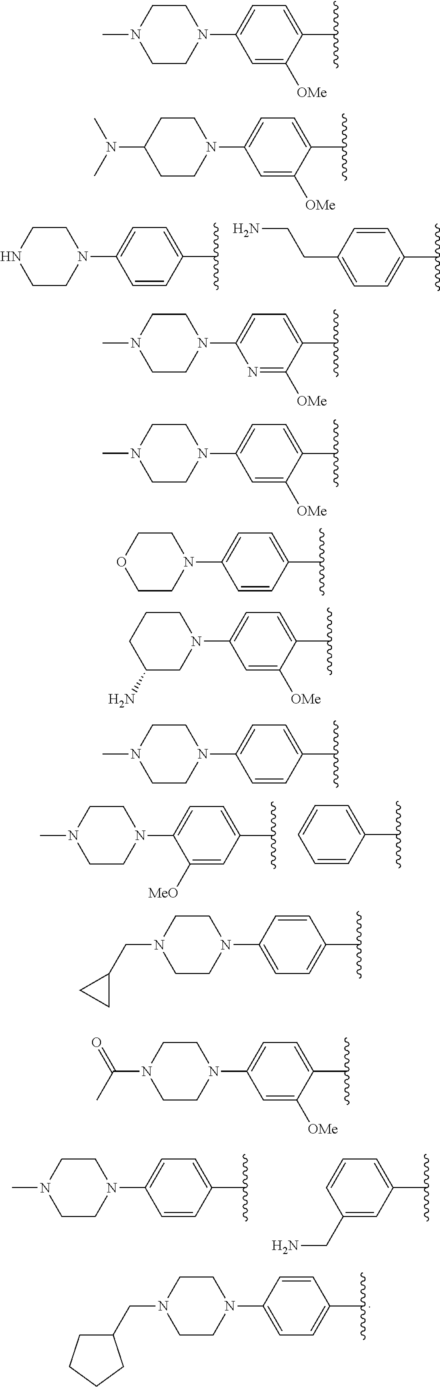

10. The compound of claim 1 wherein the substituted groups comprise 1-3 substituents selected from: halogen, —R′, —OR′, ═O, ═NR′, ═N—OR′, —NR′R″, —SR′, —SiR′R″R′″, —OC(O)R′, —C(O)R′, —CO2R′, —CONR′R″, —OC(O)NR′R″, —NR″C(O)R′, —NR′—C(O)NR″R′″, —NR′—SO2NR′″, —NR″CO2R′, —NH—C(NH2)=NH, —NR′C(NH2)=NH, —NH—C(NH2)=NR′, —S(O)R′, —SO2R′, —SO2NR′R″, —NR″SO2R, —CN and —NO2, —N3, —CH(Ph)2, perfluoro(C1-C4)alkoxy and perfluoro(C1-C4)alkyl, wherein R′, R″ and R′″ each independently refer to hydrogen, unsubstituted (C1-C8)alkyl and heteroalkyl, (C1-C8)alkyl and heteroalkyl substituted with one to three halogens, unsubstituted aryl, aryl substituted with one to three halogens, unsubstituted alkyl, alkoxy or thioalkoxy groups, or aryl-(C1-C4)alkyl groups, wherein preferred substituents are selected from: halogen, —R′, —OR′, ═O, —NR′R″, —SR′, —SiR′R″R′″, —OC(O)R′, —C(O)R′, —CO2R′, —CONR′R″, —OC(O)NR′R″, —NR″C(O)R′, —NR″CO2R′, —NR′—SO2NR″R′″, —S(O)R′, —SO2R′, —SO2NR′R″, —NR″SO2R, —CN and —NO2, perfluoro(C1-C4)alkoxy and perfluoro(C1-C4)alkyl, where R′ and R″ are as defined above.

11. The compound of claim 2 wherein the substituted groups comprise 1-3 substituents selected from: halogen, —R′, —OR′, ═O, ═NR′, ═N—OR′, —NR′R″, —SR′, —SiR′R″R′″, —OC(O)R′, —C(O)R′, —CO2R′, —CONR′R″, —OC(O)NR′R″, —NR″C(O)R′, —NR′—C(O)NR″R′″, —NR′—SO2NR′″, —NR″CO2R′, —NH—C(NH2)=NH, —NR′C(NH2)=NH, —NH—C(NH2)=NR′, —S(O)R′, —SO2R′, —SO2NR′R″, —NR″SO2R, —CN and —NO2, —N3, —CH(Ph)2, perfluoro(C1-C4)alkoxy and perfluoro(C1-C4)alkyl, wherein R′, R″ and R′″ each independently refer to hydrogen, unsubstituted (C1-C8)alkyl and heteroalkyl, (C1-C8)alkyl and heteroalkyl substituted with one to three halogens, unsubstituted aryl, aryl substituted with one to three halogens, unsubstituted alkyl, alkoxy or thioalkoxy groups, or aryl-(C1-C4)alkyl groups, wherein preferred substituents are selected from: halogen, —R′, —OR′, ═O, —NR′R″, —SR′, —SiR′R″R′″, —OC(O)R′, —C(O)R′, —CO2R′, —CONR′R″, —OC(O)NR′R″, —NR″C(O)R′, —NR″CO2R′, —NR′—SO2NR″R′″, —S(O)R′, —SO2R′, —SO2NR′R″, —NR″SO2R, —CN and —NO2, perfluoro(C1-C4)alkoxy and perfluoro(C1-C4)alkyl, where R′ and R″ are as defined above.

12. The compound of claim 3 wherein the substituted groups comprise 1-3 substituents selected from: halogen, —R′, —OR′, ═O, ═NR′, ═N—OR′, —NR′R″, —SR′, —SiR′R″R′″, —OC(O)R′, —C(O)R′, —CO2R′, —CONR′R″, —OC(O)NR′R″, —NR″C(O)R′, —NR′—C(O)NR″R′″, —NR′—SO2NR″, —NR″CO2R′, —NH—C(NH2)=NH, —NR′C(NH2)=NH, —NH—C(NH2)=NR′, —S(O)R′, —SO2R′, —SO2NR′R″, —NR″SO2R, —CN and —NO2, —N3, —CH(Ph)2, perfluoro(C1-C4)alkoxy and perfluoro(C1-C4)alkyl, wherein R′, R″ and R′″ each independently refer to hydrogen, unsubstituted (C1-C8)alkyl and heteroalkyl, (C1-C8)alkyl and heteroalkyl substituted with one to three halogens, unsubstituted aryl, aryl substituted with one to three halogens, unsubstituted alkyl, alkoxy or thioalkoxy groups, or aryl-(C1-C4)alkyl groups, wherein preferred substituents are selected from: halogen, —R′, —OR′, ═O, —NR′R″, —SR′, —SiR′R″R′″, —OC(O)R′, —C(O)R′, —CO2R′, —CONR′R″, —OC(O)NR′R″, —NR″C(O)R′, —NR″CO2R′, —NR′—SO2NR″R′″, —S(O)R′, —SO2R′, —SO2NR′R″, —NR″SO2R, —CN and —NO2, perfluoro(C1-C4)alkoxy and perfluoro(C1-C4)alkyl, where R′ and R″ are as defined above.

13. The compound of claim 4 wherein the substituted groups comprise 1-3 substituents selected from: halogen, —R′, —OR′, ═O, ═NR′, ═N—OR′, —NR′R″, —SiR′R″R′″, —OC(O)R′, —C(O)R′, —CO2R′, —CONR′R″, —OC(O)NR′R″, —NR″C(O)R′, —NR′—C(O)NR″R′″, —NR′—SO2NR′″, —NR″CO2R′, —NH—C(NH2)=NH, —NR′C(NH2)=NH, —NH—C(NH2)=NR′, —S(O)R′, —SO2R′, —SO2NR′R″, —NR″SO2R, —CN and —NO2, —N3, —CH(Ph)2, perfluoro(C1-C4)alkoxy and perfluoro(C1-C4)alkyl, wherein R′, R″ and R′″ each independently refer to hydrogen, unsubstituted (C1-C8)alkyl and heteroalkyl, (C1-C8)alkyl and heteroalkyl substituted with one to three halogens, unsubstituted aryl, aryl substituted with one to three halogens, unsubstituted alkyl, alkoxy or thioalkoxy groups, or aryl-(C1-C4)alkyl groups, wherein preferred substituents are selected from: halogen, —R′, —OR′, ═O, —NR′R″, —SR′, —SiR′R″R′″, —OC(O)R′, —C(O)R′, —CO2R′, —CONR′R″, —OC(O)NR′R″, —NR″C(O)R′, —NR″CO2R′, —NR′—SO2NR″R′″, —S(O)R′, —SO2R′, —SO2NR′R″, —NR″SO2R, —CN and —NO2, perfluoro(C1-C4)alkoxy and perfluoro(C1-C4)alkyl, where R′ and R″ are as defined above.

14. The compound of claim 1 having a structure disclosed in Table 1.

15. The compound of claim 1 having a structure disclosed in Table 2.

16. The compound of claim 1 having a structure disclosed in Table 3.

17. The compound of claim 1 that is a cathepsin C inhibitor not an epidermal growth factor receptor (EGFR) inhibitor.

18. The compound of claim 1 that is a cathepsin C inhibitor having an IC50 of less than 30, 10, 3 or 1 μM, and not an epidermal growth factor receptor (EGFR) inhibitor, wherein the EGFR IC50 is at least 3, 10, 100, or 1000 times higher than the cathepsin C IC50.

19. A pharmaceutical composition comprising a therapeutically effective amount of a compound of claim 1 and one or more pharmaceutically acceptable excipients, in unit dosage form.

20. A method of using a compound of claim 1 to inhibit cathepsin C comprising: administering to a cell or person in need thereof an effective amount of a compound of claim 1, or a prodrug thereof, or to treat inflammation comprising: administering to a cell or person in need thereof an effective amount of the compound, or a prodrug thereof, optionally further comprising the antecedent step of diagnosing the inflammation or the subsequent step of detecting a resultant amelioration of the inflammation.
